(19) United States
(12) Patent Application Publication
(10) Pub. No.: US 2021/0030888 A1
(43) Pub. Date: Feb. 4, 2021

(54) ANTI-TISSUE FACTOR ANTIBODY-DRUG CONJUGATES AND THEIR USE IN THE TREATMENT OF CANCER

(71) Applicant: Genmab A/S, COPENHAGEN V (DK)

(72) Inventors: Reshma Abdulla RANGWALA, Philadelphia, PA (US); Esther Cornelia Wilhelmina BREIJ, Utrecht (NL); David SATIJN, Utrecht (NL); Sandra VERPLOEGEN, Utrecht (NL); Jantine BAKEMA, Utrecht (NL); Oyewale O. ABIDOYE, Bellevue, WA (US); Leonardo Viana NICACIO, Redmond, WA (US); Andreas LINGNAU, Mindelheim (DE)

(21) Appl. No.: 16/978,539

(22) PCT Filed: Mar. 6, 2019
(86) PCT No.: PCT/US19/21024

§ 371 Date: Sep. 4, 2020

Related U.S. Application Data
(60) Provisional application No. 62/639,891, filed on Mar. 7, 2018.
(60) Provisional application No. 62/736,343, filed on Sep. 25, 2018.

(51) Int. Cl. A61K 47/68; C07K 16/36; A61K 9/00; A61P 35/00

(52) U.S. Cl. CPC A61K 47/6843; C07K 16/36; A61K 47/6803; C07K 2317/56; A61K 9/0019; A61P 35/00; C07K 2317/565; A61K 47/6889

(57) ABSTRACT

The invention provides methods and compositions for treating cancer, such as colorectal cancer, non-small cell lung cancer, pancreatic cancer, head and neck cancer, bladder cancer, endometrial cancer, esophageal cancer and prostate cancer, in a subject, such as by the administration of antibody-drug conjugates that bind to tissue factor (TF). The invention also provides articles of manufacture and compositions comprising said antibody drug-conjugates that bind to TF for use in treating cancer (e.g., colorectal cancer, non-small cell lung cancer, pancreatic cancer, head and neck cancer, bladder cancer, endometrial cancer, esophageal cancer and prostate cancer).

### CROSS-REFERENCE TO RELATED APPLICATIONS

This application claims priority to U.S. Provisional Application No. 62/639,891 filed Mar. 7, 2018 and U.S. Provisional Application No. 62/736,343 filed on Sep. 25, 2018 the contents of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761682000740SEQLIST.TXT, date recorded: Mar. 5, 2019, size: 6 KB).

### TECHNICAL FIELD

The present invention relates to anti-tissue factor (TF) antibody-drug conjugates and methods of using the same to treat cancer, such as colorectal cancer, non-small cell lung cancer, pancreatic cancer, head and neck cancer, bladder cancer, endometrial cancer, esophageal cancer and prostate cancer.

### BACKGROUND

Tissue factor (TF), also called thromboplastin, factor III or CD142 is a protein present in subendothelial tissue, platelets, and leukocytes necessary for the initiation of thrombin formation from the zymogen prothrombin. Thrombin formation ultimately leads to the coagulation of blood. TF enables cells to initiate the blood coagulation cascade, and it functions as the high-affinity receptor for the coagulation factor VIIa (FVIIa), a serine protease. The resulting complex provides a catalytic event that is responsible for initiation of the coagulation protease cascades by specific limited proteolysis. Unlike the other cofactors of these protease cascades, which circulate as nonfunctional precursors, TF is a potent initiator that is fully functional when expressed on cell surfaces.

TF is the cell surface receptor for the serine protease factor VIIa (FVIIa). Binding of FVIIa to TF starts signaling processes inside the cell, said signaling function playing a role in angiogenesis. Whereas angiogenesis is a normal process in growth and development, as well as in wound healing, it is also a fundamental step in the transition of tumors from a dormant state to a malignant state. When cancer cells gain the ability to produce proteins that participate in angiogenesis (i.e., angiogenic growth factors), these proteins are released by the tumor into nearby tissues, thereby stimulating new blood vessels to sprout from existing healthy blood vessels toward and into the tumor. Once new blood vessels enter the tumor, the tumor can rapidly expand its size and invade local tissue and organs. Through the new blood vessels, cancer cells may further escape into the circulation and lodge in other organs to form new tumors, also known as metastasis.

TF expression is observed in many types of cancer, and is associated with more aggressive disease. Furthermore, human TF also exists in a soluble alternatively-spliced form, asHTF. It has been found that asHTF promotes tumor growth (Hobbs et al., 2007, Thrombosis Res. 120(2):S13-S21).

In the United States, more than 1.3 million people were estimated to be living with colorectal cancer in 2014, and more than 50,000 are estimated to have died from this disease in 2017. Worldwide, approximately 10% of all non-melanoma cancers can be classified as colorectal. Though colorectal cancer mortality rates have steadily declined in recent years due in part to better screening rates for early detection, 5 year survival for patients with metastatic colorectal cancer is only 21%. The vast majority of non-operable metastatic colorectal cancer patients cannot be cured and the goal of therapy remains palliative. Systemic therapies for non-operable colorectal cancer include fluorouracil (5-FU), immunotherapy such as pembrolizumab and nivolumab, regorafenib, trifluridine-tipiracil doublet (TAS-102), and irinotecan or oxaliplatin in combination with 5-FU. More effective treatments for these later stage patients are urgently needed.

Lung cancer remains the leading cause of death from cancer in the United States, with over 155,000 deaths estimated in 2017. Treatments with curative intent for patients with early stage disease include surgery, chemotherapy, radiation therapy, or a combined modality approach. However, a majority of patients are diagnosed with advanced stage disease, which is usually incurable. Non-small cell lung cancer (NSCLC) represents up to 80% of all lung cancers. Within the subtypes of NSCLC, squamous cell carcinoma (SCC/NSCLC) represents approximately 30% of NSCLC. Systemic therapies used in the metastatic setting for SCC/NSCLC have shown limited benefit and are primarily aimed at prolonging survival and maintaining the quality of life for as long as possible, while minimizing side effects due to treatment. First line treatment for patients with SCC/NSCLC whose tumors do not express high levels of PD-L1 include a platinum-based chemotherapy doublet that does not contain pemetrexed, anti-VEGF antibody, or an anti-EGFR antibody necitumumab in combination with gemcitabine and cisplatin. Patients with at least 50% tumor cell staining for PD-L1 are offered first-line treatment with the anti-PD-1 inhibitor pembrolizumab. Patients who progress on an initial combination chemotherapy regimen may receive an anti-PD-1 or PD-L1 antibody, and combination chemotherapy is considered for patients whose disease has progressed after receiving PD-1/L1 inhibitors. New classes of therapy are urgently needed that can provide meaningful benefit to SCC/NSCLC patients.

Pancreatic cancer is considered a “silent killer” because patients often do not feel symptoms until their disease has advanced and spread—in the US, 52% of patients had metastatic disease at diagnosis in 2017. More than 53,000 cases are estimated to have been diagnosed in the US in 2017, with over 43,000 deaths. Five year survival for people with metastatic pancreatic cancer remains a dismal 8% in the US and may be as low as 4% worldwide. Most patients diagnosed with pancreatic cancer succumb to the disease within the first year. Surgical resection offers the only chance of cure. However, only 15% to 20% of patients have resectable disease at initial diagnosis; the majority have either locally advanced or metastatic cancer. Metastatic pancreatic cancer patients have very few effective treatment options and are often treated only with palliative care. First line combination treatments include FOLFIRINOX or nab-paclitaxel plus gemcitabine. Second line and later treatments offer limited efficacy with significant treatment-related toxicity. Preferred regimens in this group include liposomal irinotecan (Onivyde) with 5-FU/leucovorin, FOLFOX, and gemcitabine in combination with nab-paclitaxel, erlotinib, or bevacizumab. Enrollment in available clinical trials is a preferred option for patients with advanced exocrine pancreatic adenocarcinoma, if available, due to the significant unmet medical need in this disease.

Head and neck cancers make up approximately 3% of cancers in the United States. Over 63,000 cases are estimated to have been diagnosed in 2017 and more than 13,000 patients died from this disease. Though human papilloma virus (HPV) infection also appears to contribute to head and neck cancers. More than 90-95% of oral and nasopharyngeal cancers are of squamous histology. Surgical resection, radiotherapy, and/or chemoradiation are frequently recommended for patients with early-stage or localized disease. Palliative chemotherapy, immunotherapy and/or supportive care are the most appropriate options for patients with locally recurrent or metastatic disease that are not amenable to definitive therapy. Platinum-based regimens are the preferred standard of care treatment for patients with recurrent or de novo metastatic squamous cell carcinoma of the head and neck (SCCHN). Cetuximab in combination with a platinum/5-FU regimen has demonstrated clinically meaningful benefits compared to platinum/5-FU alone. For patients progressing on first line treatment, second line treatment is with single agent chemotherapy, targeted therapy, or a checkpoint inhibitor such as nivolumab or pembrolizumab. Overall, there is a great unmet medical need for patients with SCCHN that have progressed after first line platinum combination therapy followed by second line PD-1 therapy.

Bladder cancer is the sixth most common cancer in the United States, with an estimated 76,960 new cases diagnosed in 2016. Of these patients, 16,390 deaths were estimated to have occurred, with men being more likely to be affected than women. The 5-year relative survival rate for all stages combined is 77%. However, survival rates depend on many factors, including the histology and stage of bladder cancer diagnosed. For patients with bladder cancer that is invasive but not yet spread outside the bladder, the 5-year survival rate is 70%. For patients with bladder cancer that extends through the bladder to the surrounding tissue and/or organs, the 5-year survival rate is 34%. A cisplatin-based chemotherapy regimen followed by surgical removal of the bladder or radiation therapy and concomitant chemotherapy is currently the standard treatment for patients with invasive bladder cancer. More effective treatments for bladder cancer, particularly for patients with advanced or metastatic bladder cancer, are urgently needed.

Endometrial cancer is the most common gynecologic malignancy in the United States, accounting for 6% of cancers in women. In 2017, an estimated 61,380 women were diagnosed with endometrial cancer, and approximately 11,000 died from this disease. From 1987 to 2008, there was a 50% increase in the incidence of endometrial cancer, with an approximate 300% increase in the number of associated deaths. Endometrial adenocarcinomas can be classified into two histologic categories-type 1 or type 2. Approximately 70-80% of new cases are classified as type 1 endometrial carcinomas, which are of endometrioid histology, lower grade, and often confined to the uterus at diagnosis. These tumors are estrogen-mediated, and often, women diagnosed with type 1 endometrial carcinomas are obese, with excess endogenous estrogen production. Type 1 carcinomas (estrogen dependent) have high rates of K-ras and PTEN loss or mutation, as well as defects in mismatch repair genes, which lead to microsatellite instability (MSI). Type 2 (non-estrogen dependent) carcinomas are higher-grade adenocarcinomas and are of non-endometrioid histology, occurring in older, leaner women, although an association with increasing body mass index (BMI) has been observed. Type 2 cancers have p53 mutations, may have overexpression of human epidermal growth factor receptor 2 (HER-2/neu), and show aneuploidy. Although there are many chemotherapeutic and targeted therapy agents approved for ovarian, fallopian tube and primary peritoneal cancers, since the 1971 approval of megestrol acetate for the palliative treatment of advanced endometrial cancer, only pembrolizumab has been Food and Drug Administration (FDA)-approved for high microsatellite instability (MSI-H) or mismatch repair deficient (dMMR) endometrial cancer; this highlights the need for new therapies to treat advanced, recurrent, metastatic endometrial cancer.

Esophageal cancer is the sixth leading cause of cancer-related mortality worldwide due to its overall poor prognosis. The global age-standardized incidence rate of esophageal squamous cell carcinoma (ESCC) is 1.4-13.6 per 100,000 people. Esophageal cancer is estimated to be responsible for 15,690 deaths and 16,940 new cases in the United States in 2016. The majority of patients present with locally advanced or systemic disease and outcomes remain poor despite advances in treatment. More effective treatments for these patients with locally advanced or systemic disease are urgently needed.

Prostate cancer is the most common non-cutaneous malignancy in males, with a projected 161,360 incident cases and 26,730 deaths estimated in the United States in 2017 alone. Curative modalities for localized prostate cancer include surgery and/or radiation therapy, with or without androgen deprivation therapy. While contemporary treatment methods, such as intensity-modulated radiotherapy, are used to deliver radiation with high accuracy, defining the position and the extent of the tumor is still quite challenging. Other issues in the treatment of the radiotherapy patient include the choice of the radiotherapy technique (hypo- or standard fractionation) and the use and length of androgen deprivation therapy. More effective treatments are needed, especially for patients with advanced and metastatic prostate cancer.

The present invention meets the need for improved treatment of colorectal cancer, non-small cell lung cancer, pancreatic cancer, head and neck cancer, bladder cancer, endometrial cancer, esophageal cancer and prostate cancer by providing highly specific and effective anti-TF antibody-drug conjugates.

All references cited herein, including patent applications, patent publications, and scientific literature, are herein incorporated by reference in their entirety, as if each individual reference were specifically and individually indicated to be incorporated by reference.

### SUMMARY

Provided herein are methods of treating cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof, wherein the antibody-drug conjugate is administered at a dose ranging from about 1.5 mg/kg to about 2.1 mg/kg, and wherein the cancer is selected from the group consisting of colorectal cancer, non-small cell lung cancer, pancreatic cancer, head and neck cancer, bladder cancer, endometrial cancer, esophageal cancer and prostate cancer. In some embodiments, the antibody-drug conjugate is administered at a dose of about 2.0 mg/kg. In some embodiments, the antibody-drug conjugate is administered at a dose of 2.0 mg/kg. In some of any of the embodiments herein, the antibody-drug conjugate is administered once about every 1 week, 2 weeks, 3 weeks or 4 weeks. In some of any of the embodiments herein, the antibody-drug conjugate is administered once about every 3 weeks. In some of any of the embodiments herein, the subject has been previously treated with one or more therapeutic agents and did not respond to the treatment, wherein the one or more therapeutic agents is not the antibody-drug conjugate. In some of any of the embodiments herein, the subject has been previously treated with one or more therapeutic agents and relapsed after the treatment, wherein the one or more therapeutic agents is not the antibody-drug conjugate. In some of any of the embodiments herein, the subject has been previously treated with one or more therapeutic agents and has experienced disease progression during treatment, wherein the one or more therapeutic agents is not the antibody-drug conjugate. In some of any of the embodiments herein, the cancer is colorectal cancer. In some of any of the embodiments herein, the subject received prior systemic therapy and experienced disease progression on or after the systemic therapy. In some of any of the embodiments herein, the subject received 1, 2 or 3 rounds of prior systemic therapy. In some of any of the embodiments herein, the colorectal cancer is non-operable. In some of any of the embodiments herein, the subject has been previously treated with one or more agents selected from the group consisting of fluoropyrimidine, oxaliplatin, irinotecan and bevacizumab. In some of any of the embodiments herein, the subject has been previously treated with one or more agents selected from the group consisting of cetuximab, panitumab and a checkpoint inhibitor. In some of any of the embodiments herein, the cancer is non-small cell lung cancer. In some of any of the embodiments herein, the non-small cell lung cancer is squamous cell carcinoma. In some of any of the embodiments herein, the non-small cell lung cancer has predominant squamous histology. In some of any of the embodiments herein, greater than 85% of the non-small cell lung cancer cells have squamous histology. In some of any of the embodiments herein, the non-small cell lung cancer is adenocarcinoma. In some of any of the embodiments herein, the subject received prior systemic therapy and experienced disease progression on or after the systemic therapy. In some of any of the embodiments herein, the subject received 1 or 2 rounds of prior systemic therapy. In some of any of the embodiments herein, the subject has been previously treated with one or more agents selected from the group consisting of a platinum-based therapy and a checkpoint inhibitor. In some of any of the embodiments herein, the cancer is pancreatic cancer. In some of any of the embodiments herein, the pancreatic cancer is exocrine pancreatic adenocarcinoma. In some of any of the embodiments herein, the pancreatic cancer has predominant adenocarcinoma histology. In some of any of the embodiments herein, greater than 85% of the pancreatic cancer cells have adenocarcinoma histology. In some of any of the embodiments herein, the subject received prior systemic therapy and experienced disease progression on or after the systemic therapy. In some of any of the embodiments herein, the subject received 1 round of prior systemic therapy. In some of any of the embodiments herein, the subject has been previously treated with one or more agents selected from the group consisting of gemcitabine and 5-fluorouracil. In some of any of the embodiments herein, the pancreatic cancer is not resectable. In some of any of the embodiments herein, the cancer is head and neck cancer. In some of any of the embodiments herein, the head and neck cancer is squamous cell carcinoma. In some of any of the embodiments herein, the subject received prior systemic therapy and experienced disease progression on or after the systemic therapy. In some of any of the embodiments herein, the subject received 1 or 2 rounds of prior systemic therapy. In some of any of the embodiments herein, the subject has been previously treated with one or more agents selected from the group consisting of a platinum-based therapy and a checkpoint inhibitor. In some of any of the embodiments herein, the subject has been previously treated with an anti-epithelial growth factor receptor therapy. In some of any of the embodiments herein, the cancer is bladder cancer. In some of any of the embodiments herein, the subject received prior systemic therapy and experienced disease progression on or after the systemic therapy. In some of any of the embodiments herein, the subject received 1, 2 or 3 rounds of prior systemic therapy. In some of any of the embodiments herein, the subject has been previously treated with a platinum-based therapy. In some of any of the embodiments herein, the subject has previously undergone surgery or radiation therapy for the bladder cancer. In some of any of the embodiments herein, the cancer is endometrial cancer. In some of any of the embodiments herein, the subject received prior systemic therapy and experienced disease progression on or after the systemic therapy. In some of any of the embodiments herein, the subject received 1, 2 or 3 rounds of prior systemic therapy. In some of any of the embodiments herein, the subject has been previously treated with one or more agents selected from the group consisting of a platinum-based therapy, hormone therapy, and a checkpoint inhibitor. In some of any of the embodiments herein, the subject has previously been treated with doxorubicin. In some of any of the embodiments herein, the subject has previously been treated with paclitaxel. In some of any of the embodiments herein, the subject has previously undergone surgery or radiation therapy for the endometrial cancer. In some of any of the embodiments herein, the cancer is esophageal cancer. In some of any of the embodiments herein, the subject received prior systemic therapy and experienced disease progression on or after the systemic therapy. In some of any of the embodiments herein, the subject received 1, 2 or 3 rounds of prior systemic therapy. In some of any of the embodiments herein, the subject has been previously treated with one or more agents selected from the group consisting of a platinum-based therapy and a checkpoint inhibitor. In some of any of the embodiments herein, the subject has been previously treated with one or more agents selected from the group consisting of ramucirumab, paclitaxel, 5-fluorouracil, docetaxel, irinotecan, capecitabine and trastuzumab. In some of any of the embodiments herein, the subject has previously undergone surgery, radiation therapy or endoscopic mucosal resection for the esophageal cancer. In some of any of the embodiments herein, the cancer is prostate cancer. In some of any of the embodiments herein, the subject received prior systemic therapy and experienced disease progression on or after the systemic therapy. In some of any of the embodiments herein, the subject received 1, 2 or 3 rounds of prior systemic therapy. In some of any of the embodiments herein, the prostate cancer is castration-resistant prostate cancer. In some of any of the embodiments herein, the subject experienced bone metastases. In some of any of the embodiments herein, the subject has been previously treated with one or more agents selected from the group consisting of androgen deprivation therapy, a luteinizing hormone-releasing hormone agonist, a luteinizing hormone-releasing hormone antagonist, a CYP17 inhibitor, and an anti-androgen. In some of any of the embodiments herein, the subject has been previously treated with one or more agents selected from the group consisting of docetaxel, prednisone and cabazitaxel. In some of any of the embodiments herein, the subject has previously undergone surgery or radiation therapy for the prostate cancer. In some of any of the embodiments herein, the cancer is an advanced stage cancer. In some of any of the embodiments herein, the advanced stage cancer is a stage 3 or stage 4 cancer. In some of any of the embodiments herein, the advanced stage cancer is metastatic cancer. In some of any of the embodiments herein, the cancer is recurrent cancer. In some of any of the embodiments herein, the subject received prior treatment with standard of care therapy for the cancer and failed the prior treatment. In some of any of the embodiments herein, the monomethyl auristatin is monomethyl auristatin E (MMAE). In some of any of the embodiments herein, the anti-TF antibody or antigen-binding fragment thereof of the antibody-drug conjugate is a monoclonal antibody or a monoclonal antigen-binding fragment thereof. In some of any of the embodiments herein, the anti-TF antibody or antigen-binding fragment thereof of the antibody-drug conjugate comprises a heavy chain variable region and a light chain variable region, wherein the heavy chain variable region comprises:

(i) a CDR-H1 comprising the amino acid sequence of SEQ ID NO:1;

(ii) a CDR-H2 comprising the amino acid sequence of SEQ ID NO:2; and

(iii) a CDR-H3 comprising the amino acid sequence of SEQ ID NO:3; and wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6.

In some of any of the embodiments herein, the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8. In some of any of the embodiments herein, the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8. In some of any of the embodiments herein, the anti-TF antibody of the antibody-drug conjugate is tisotumab. In some of any of the embodiments herein, the antibody-drug conjugate further comprises a linker between the anti-TF antibody or antigen-binding fragment thereof and the monomethyl auristatin. In some of any of the embodiments herein, the linker is a cleavable peptide linker. In some of any of the embodiments herein, the cleavable peptide linker has a formula: -MC-vc-PAB-, wherein:

a) MC is:

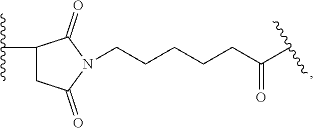 [SMILES: *C(=O)CCCCCN1C(=O)CC(*)C1=O]

b) vc is the dipeptide valine-citrulline, and

c) PAB is:

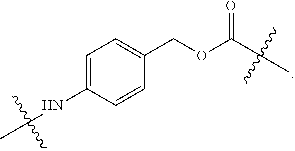 [SMILES: *Nc1ccc(COC(*)=O)cc1]

In some of any of the embodiments herein, the linker is attached to sulphydryl residues of the anti-TF antibody obtained by partial reduction or full reduction of the anti-TF antibody or antigen-binding fragment thereof. In some of any of the embodiments herein, the linker is attached to monomethyl auristatin E (MMAE), wherein the antibody-drug conjugate has the following structure:

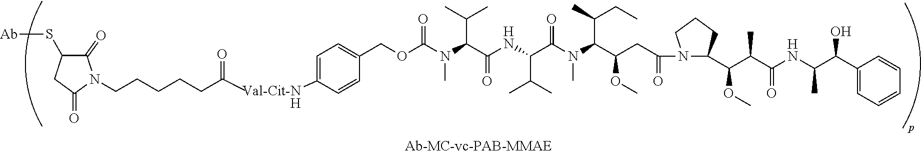

wherein p denotes a number from 1 to 8, S represents a sulphydryl residue of the anti-TF antibody, and Ab designates the anti-TF antibody or antigen-binding fragment thereof. In some of any of the embodiments herein, the average value of p in a population of the antibody-drug conjugates is about 4. In some of any of the embodiments herein, the antibody-drug conjugate is tisotumab vedotin. In some of any of the embodiments herein, the route of administration for the antibody-drug conjugate is intravenous. In some of any of the embodiments herein, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express TF. In some of any of the embodiments herein, the one or more therapeutic effects in the subject is improved after administration of the antibody-drug conjugate relative to a baseline. In some of any of the embodiments herein, the one or more therapeutic effects is selected from the group consisting of: size of a tumor derived from the cancer, objective response rate, duration of response, time to response, progression free survival, overall survival and prostate-specific antigen (PSA) level. In some of any of the embodiments herein, the subject exhibits a reduction in PSA level in a blood sample from the subject by at least about 5%, at least about 10%, at least about 15%, at least about 20%, at least about 25%, at least about 30%, at least about 35%, at least about 40%, at least about 45%, at least about 50%, at least about 60%, at least about 70%, or at least about 80% relative to the PSA level in a blood sample obtained from the subject before administration of the antibody-drug conjugate. In some of any of the embodiments herein, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate. In some of any of the embodiments herein, the objective response rate is at least about 20%, at least about 25%, at least about 30%, at least about 35%, at least about 40%, at least about 45%, at least about 50%, at least about 60%, at least about 70%, or at least about 80%. In some of any of the embodiments herein, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some of any of the embodiments herein, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some of any of the embodiments herein, the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some of any of the embodiments herein, the subject has one or more adverse events and is further administered an additional therapeutic agent to eliminate or reduce the severity of the one or more adverse events. In some of any of the embodiments herein, the subject is at risk of developing one or more adverse events and is further administered an additional therapeutic agent to prevent or reduce the severity of the one or more adverse events. In some of any of the embodiments herein, the one or more adverse events is anemia, abdominal pain, hypokalemia, hyponatremia, epistaxis, fatigue, nausea, alopecia, conjunctivitis, constipation, decreased appetite, diarrhea, vomiting, peripheral neuropathy, or general physical health deterioration. In some of any of the embodiments herein, the one or more adverse events is a grade 3 or greater adverse event. In some of any of the embodiments herein, the one or more adverse events is a serious adverse event. In some of any of the embodiments herein, the one or more adverse events is conjunctivitis and/or keratitis and the additional agent is a preservative-free lubricating eye drop, an ocular vasoconstrictor and/or a steroid eye drop. In some of any of the embodiments herein, the antibody-drug conjugate is administered as a monotherapy. In some of any of the embodiments herein, the subject is a human. In some of any of the embodiments herein, the antibody-drug conjugate is in a pharmaceutical composition comprising the antibody-drug conjugate and a pharmaceutical acceptable carrier.

Also provided herein are kits comprising:

(a) a dosage ranging from about 0.9 mg/kg to about 2.1 mg/kg of an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof; and

(b) instructions for using the antibody drug conjugate according to some of any of the embodiments herein.

### BRIEF DESCRIPTION OF THE DRAWINGS

FIG. 1 is a diagram showing the mechanism of action (MOA) of the antibody-drug conjugate tisotumab vedotin.

FIG. 2A-2B show dose-dependent anti-tumor effects of single-dose tisotumab vedotin treatment in a NCI-H441 cell line-derived (CDX) mouse xenograft model. FIG. 2A shows tumor growth of the NCI-H441 xenografts after treatment with different doses of tisotumab vedotin, isotype control antibody (IgG1-b12), or isotype control ADC (IgG-b12-vcMMAE). Mean and standard of error of the mean (SEM) of each group is shown at each time point. FIG. 2B shows mean tumor size in each mouse on day 47. Mean and SEM of each group are indicated. Differences among the groups were analyzed by one-way ANOVA. Statistically significant differences are indicated as follows: *: p<0.05; **: p<0.01; ***: p<0.001.

FIG. 3 shows anti-tumor effects of tisotumab vedotin treatment in a squamous cell lung carcinoma patient-derived xenograft (PDX) mouse model LXFE 690. Mean and SEM of tumor size of the LXFE 690 xenografts at each time point in groups treated with two doses of tisotumab vedotin at 4 mg/kg, IgG1-b12 or IgG1b12-vcMMAE are shown.

FIG. 4A-4B show dose-dependent anti-tumor effects of tisotumab vedotin treatment in a HPAF II CDX mouse model. FIG. 4A shows tumor growth of the HPAF II xenografts after treatment with tisotumab vedotin, or IgG1-b12. Mean and SEM of each group is shown at each time point. FIG. 4B shows mean tumor size in each mouse on day 25. Mean and SEM of each group are indicated. Differences among the groups were analyzed by one-way ANOVA. Statistically significant differences versus the IgG1-b12 group are indicated as follows: *: p<0.05; **: p<0.01; ***: p<0.001.

FIG. 5 shows anti-tumor effects of tisotumab vedotin treatment in a pancreatic cancer PDX mouse model PAXF 1657. Mean and SEM of tumor size of the PAXF 1657 xenografts at each time point in groups treated with two doses of tisotumab vedotin at 4 mg/kg, IgG1-b12 or IgG1b12-vcMMAE are shown.

FIG. 6 shows anti-tumor effects of tisotumab vedotin treatment in a SCCHN cancer CDX mouse model FaDu. Mean and SEM of tumor size of the FaDu xenografts at each time point in groups treated with three doses of tisotumab vedotin, PBS or IgG1b12-vcMMAE are shown.

FIG. 7 shows anti-tumor effects of tisotumab vedotin treatment in the BXF 1036 bladder cancer patient-derived xenograft model. Average tumor size in the BXF 1036 patient-derived xenograft model in athymic nude mice after treatment with tisotumab vedotin (0.5, 1, 2 or 4 mg/kg), an isotype control ADC (IgG1-b12-MMAE, 4 mg/kg) or an isotype control IgG (IgG1-b12, 4 mg/kg). Tumor size was assessed by caliper measurement. Error bars indicate standard error of the mean (S.E.M.).

FIG. 8 shows anti-tumor effects of tisotumab vedotin treatment in the BXF 1036 bladder cancer patient-derived xenograft model. Tumor size in individual mice in the BXF 1036 patient-derived xenograft model in athymic nude mice, on day 31 after treatment with tisotumab vedotin (0.5, 1, 2 or 4 mg/kg), an isotype control ADC (IgG1-b12-MMAE, 4 mg/kg) or an isotype control IgG (IgG1-b12, 4 mg/kg). Tumor size was assessed by caliper measurement. Symbols represent individual mice, horizontal lines represent mean tumor size per treatment group and error bars represent standard error of the mean (S.E.M.)

FIG. 9 shows anti-tumor effects of tisotumab vedotin treatment in an esophageal cancer patient-derived xenograft model in nude mice. Average tumor size in the ES0195 patient-derived xenograft model in nude mice after treatment with tisotumab vedotin (4 mg/kg), an isotype control ADC (IgG1-b12-MMAE, 4 mg/kg) or an isotype control IgG (IgG1-b12, 4 mg/kg). Tumor size was assessed by caliper measurement. Error bars indicate standard error of the mean (S.E.M.).

FIG. 10 shows anti-tumor effects of tisotumab vedotin treatment in a PAXF1657 pancreatic cancer patient-derived xenograft model in nude mice. Average tumor size in the PAXF 1657 patient-derived xenograft model in athymic nude mice after treatment with tisotumab vedotin (4 mg/kg), an isotype control ADC (IgG1-b12-MMAE, 4 mg/kg) or an isotype control IgG (IgG1-b12, 4 mg/kg). Tumor size was assessed by caliper measurement. Error bars indicate standard error of the mean (S.E.M.).

FIG. 11 shows anti-tumor effects of tisotumab vedotin treatment in a PA5415 pancreatic cancer patient-derived xenograft model in NOD-SCID mice. Average tumor size in the PA5415 patient-derived xenograft model in NOD-SCID mice after treatment with tisotumab vedotin (0.5, 1 or 2 mg/kg), an isotype control ADC (IgG-b12-MMAE, 2 mg/kg) or an isotype control IgG (IgG1-b12, 2 mg/kg). Tumor size was assessed by caliper measurement. Error bars indicate standard error of the mean (S.E.M.).

FIG. 12 shows anti-tumor effects of tisotumab vedotin treatment in PA5415 pancreatic cancer patient-derived xenograft model in NOD-SCID mice. Tumor-free survival after treatment with tisotumab vedotin (0.5, 1 or 2 mg/kg), an isotype control ADC (IgG1-b12-MMAE, 2 mg/kg) or an isotype control IgG (IgG1-b12, 2 mg/kg). Tumor size was assessed by caliper measurement. A tumor size of 500 mm³ was used as a cut-off for tumor progression.

FIG. 13 shows anti-tumor effects of tisotumab vedotin treatment in a diverse panel of colorectal cancer (CRC) patient-derived xenograft (PDX) models in NOD-SCID mice. Responding models (R) were defined as models showing ΔT/ΔC<10% (tumor stasis or tumor regression), and non-responding models were defined as ΔT/ΔC>70%. The models that could not be classified as responder or non-responder (10%<ΔT/ΔC<70%), were classified as intermediate.

FIG. 14 shows anti-tumor effects of tisotumab vedotin treatment in a diverse panel of colorectal cancer (CRC) patient-derived xenograft (PDX) models in NOD-SCID mice. Responding models (R) were defined as models showing ΔT/ΔC<10% (tumor stasis or tumor regression), and non-responding models were defined as ΔT/ΔC>70%. The models that could not be classified as responder or non-responder (10%<ΔT/ΔC<70%), were classified as intermediate.

FIG. 15 shows average TF mRNA expression levels in PDX models classified as responder, non-responder or intermediate.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the invention described herein include “comprising,” “consisting,” and “consisting essentially of” aspects and embodiments.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “tissue factor”, “TF”, “CD142”, “tissue factor antigen”, “TF antigen” and “CD142 antigen” are used interchangeably herein, and, unless specified otherwise, include any variants, isoforms and species homologs of human tissue factor which are naturally expressed by cells or are expressed on cells transfected with the tissue factor gene. In some embodiments, tissue factor comprises the amino acid sequence found under Genbank accession NP_001984.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region (C_{H} or CH). The heavy chain constant region typically is comprised of three domains, C_{H}1, C_{H}2, and C_{H}3. The heavy chains are generally inter-connected via disulfide bonds in the so-called “hinge region.” Each light chain typically is comprised of a light chain variable region (abbreviated herein as V_{L} or VL) and a light chain constant region (C_{L} or CL). The light chain constant region typically is comprised of one domain, C_{L}. The CL can be of κ (kappa) or λ (lambda) isotype. The terms “constant domain” and “constant region” are used interchangeably herein. Unless stated otherwise, the numbering of amino acid residues in the constant region is according to the EU-index as described in Kabat et al., Sequences of Proteins of Immunological Interest, 5th Ed. Public Health Service, National Institutes of Health, Bethesda, Md. (1991). An immunoglobulin can derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

The term “variable region” or “variable domain” refers to the domain of an antibody heavy or light chain that is involved in binding the antibody to antigen. The variable regions of the heavy chain and light chain (V_{H} and V_{L}, respectively) of a native antibody may be further subdivided into regions of hypervariability (or hypervariable regions, which may be hypervariable in sequence and/or form of structurally defined loops), also termed complementarity-determining regions (CDRs), interspersed with regions that are more conserved, termed framework regions (FRs). The terms “complementarity determining regions” and “CDRs,” synonymous with “hypervariable regions” or “HVRs” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Within each V_{H} and V_{L}, three CDRs and four FRs are typically arranged from amino-terminus to carboxy-terminus in the following order: FR1, CDR1, FR2, CDR2, FR3, CDR3, FR4 (See also Chothia and Lesk J. Mot. Biol., 195, 901-917 (1987)).

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 min, at least about 45 min, at least about one hour (h), at least about two hours, at least about four hours, at least about eight hours, at least about 12 hours (h), about 24 hours or more, about 48 hours or more, about three, four, five, six, seven or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The constant regions of the antibodies (Abs) may mediate the binding of the immunoglobulin to host tissues or factors, including various cells of the immune system (such as effector cells) and components of the complement system such as Clq, the first component in the classical pathway of complement activation. An antibody may also be a bispecific antibody, diabody, multispecific antibody or similar molecule.

The term “monoclonal antibody” as used herein refers to a preparation of antibody molecules that are recombinantly produced with a single primary amino acid sequence. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

An “isolated antibody” refers to an antibody that is substantially free of other antibodies having different antigenic specificities (e.g., an isolated antibody that binds specifically to TF is substantially free of antibodies that bind specifically to antigens other than TF). An isolated antibody that binds specifically to TF can, however, have cross-reactivity to other antigens, such as TF molecules from different species. Moreover, an isolated antibody can be substantially free of other cellular material and/or chemicals. In one embodiment, an isolated antibody includes an antibody conjugate attached to another agent (e.g., small molecule drug). In some embodiments, an isolated anti-TF antibody includes a conjugate of an anti-TF antibody with a small molecule drug (e.g., MMAE or MMAF).

A “human antibody” (HuMAb) refers to an antibody having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “chimeric antibody” as used herein, refers to an antibody wherein the variable region is derived from a non-human species (e.g. derived from rodents) and the constant region is derived from a different species, such as human. Chimeric antibodies may be generated by antibody engineering. “Antibody engineering” is a term used generic for different kinds of modifications of antibodies, and which is a well-known process for the skilled person. In particular, a chimeric antibody may be generated by using standard DNA techniques as described in Sambrook et al., 1989, Molecular Cloning: A laboratory Manual, New York: Cold Spring Harbor Laboratory Press, Ch. 15. Thus, the chimeric antibody may be a genetically or an enzymatically engineered recombinant antibody. It is within the knowledge of the skilled person to generate a chimeric antibody, and thus, generation of the chimeric antibody according to the present invention may be performed by other methods than described herein. Chimeric monoclonal antibodies for therapeutic applications are developed to reduce antibody immunogenicity. They may typically contain non-human (e.g. murine) variable regions, which are specific for the antigen of interest, and human constant antibody heavy and light chain domains. The terms “variable region” or “variable domains” as used in the context of chimeric antibodies, refers to a region which comprises the CDRs and framework regions of both the heavy and light chains of the immunoglobulin.

An “anti-antigen antibody” refers to an antibody that binds to the antigen. For example, an anti-TF antibody is an antibody that binds to the antigen TF.

An “antigen-binding portion” or antigen-binding fragment” of an antibody refers to one or more fragments of an antibody that retain the ability to bind specifically to the antigen bound by the whole antibody. Examples of antibody fragments (e.g., antigen-binding fragment) include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows:

100 times the fraction X/Y

where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % sequence identity of A to B will not equal the % sequence identity of B to A.

As used herein, the terms “binding”, “binds” or “specifically binds” in the context of the binding of an antibody to a pre-determined antigen typically is a binding with an affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less when determined by for instance BioLayer Interferometry (BLI) technology in a Octet HTX instrument using the antibody as the ligand and the antigen as the analyte, and wherein the antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} of binding to a non-specific antigen (e.g., BSA, casein) other than the predetermined antigen or a closely related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (that is, the antibody is highly specific).

The term “K_{D}” (M), as used herein, refers to the dissociation equilibrium constant of a particular antibody-antigen interaction. Affinity, as used herein, and K_{D} are inversely related, that is that higher affinity is intended to refer to lower K_{D}, and lower affinity is intended to refer to higher K_{D}.

The term “ADC” refers to an antibody-drug conjugate, which in the context of the present invention refers to an anti-TF antibody, which is coupled to a drug moiety (e.g., MMAE or MMAF) as described in the present application.

The abbreviations “vc” and “val-cit” refer to the dipeptide valine-citrulline.

The abbreviation “PAB” refers to the self-immolative spacer:

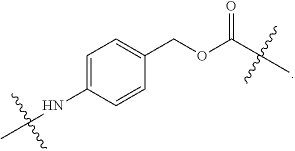 [SMILES: *Nc1ccc(COC(*)=O)cc1]

The abbreviation “MC” refers to the stretcher maleimidocaproyl:

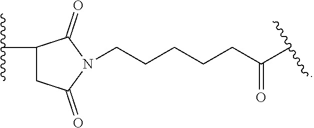 [SMILES: *C(=O)CCCCCN1C(=O)CC(*)C1=O]

The term “Ab-MC-vc-PAB-MMAE” refers to an antibody conjugated to the drug MMAE through a MC-vc-PAB linker.

A “platinum-based therapy” refers to treatment with a platinum-based agent. A “platinum-based agent” refers to a molecule or a composition comprising a molecule containing a coordination complex comprising the chemical element platinum and useful as a chemotherapy drug. Platinum-based agents generally act by inhibiting DNA synthesis and some have alkylating activity. Platinum-based agents encompass those that are currently being used as part of a chemotherapy regimen, those that are currently in development, and those that may be developed in the future.

A “cancer” refers to a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

A therapeutically effective amount of a drug (e.g., an anti-TF antibody-drug conjugate) includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

As used herein, “subtherapeutic dose” means a dose of a therapeutic compound (e.g., an anti-TF antibody-drug conjugate) that is lower than the usual or typical dose of the therapeutic compound when administered alone for the treatment of a hyperproliferative disease (e.g., cancer).

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” or “ORR” refers to the sum of complete response (CR) rate and partial response (PR) rate.

As used herein, “overall survival” or “OS” refers to the percentage of individuals in a group who are likely to be alive after a particular duration of time.

The term “weight-based dose”, as referred to herein, means that a dose administered to a patient is calculated based on the weight of the patient. For example, when a patient with 60 kg body weight requires 2 mg/kg of an anti-TF antibody-drug conjugate, one can calculate and use the appropriate amount of the anti-TF antibody-drug conjugate (i.e., 120 mg) for administration.

The use of the term “flat dose” with regard to the methods and dosages of the disclosure means a dose that is administered to a patient without regard for the weight or body surface area (BSA) of the patient. The flat dose is therefore not provided as a mg/kg dose, but rather as an absolute amount of the agent (e.g., the anti-TF antibody-drug conjugate). For example, a 60 kg person and a 100 kg person would receive the same dose of an antibody-drug conjugate (e.g., 240 mg of an anti-TF antibody-drug conjugate).

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis -(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Administering” refers to the physical introduction of a therapeutic agent to a subject, using any of the various methods and delivery systems known to those skilled in the art. Exemplary routes of administration for the anti-TF antibody-drug conjugate include intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The terms “baseline” or “baseline value” used interchangeably herein can refer to a measurement or characterization of a symptom before the administration of the therapy (e.g., an antibody-drug conjugate as described herein) or at the beginning of administration of the therapy. The baseline value can be compared to a reference value in order to determine the reduction or improvement of a symptom of a TF-associated disease contemplated herein (e.g., colorectal cancer, non-small cell lung cancer, pancreatic cancer and head and neck cancer). The terms “reference” or “reference value” used interchangeably herein can refer to a measurement or characterization of a symptom after administration of the therapy (e.g., an antibody-drug conjugate as described herein). The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

The term “monotherapy” as used herein means that the antibody drug conjugate is the only anti-cancer agent administered to the subject during the treatment cycle. Other therapeutic agents, however, can be administered to the subject. For example, anti-inflammatory agents or other agents administered to a subject with cancer to treat symptoms associated with cancer, but not the underlying cancer itself, including, for example inflammation, pain, weight loss, and general malaise, can be administered during the period of monotherapy.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically important”
Requires inpatient hospitalization or prolongation of existing hospitalization, excluding the following: 1) routine treatment or monitoring of the underlying disease, not associated with any deterioration in condition, 2) elective or pre-planned treatment for a pre-existing condition that is unrelated to the indication under study and has not worsened since signing the informed consent, and social reasons and respite care in the absence of any deterioration in the patient's general condition.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “once about every week,” “once about every two weeks,” or any other similar dosing interval terms as used herein mean approximate numbers. “Once about every week” can include every seven days one day, i.e., every six days to every eight days. “Once about every two weeks” can include every fourteen days two days, i.e., every twelve days to every sixteen days. “Once about every three weeks” can include every twenty-one days±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Antibody-Drug Conjugates

The present invention provides an anti-TF antibody-drug conjugate that binds to TF for use in the treatment of colorectal cancer, non-small cell lung cancer, pancreatic cancer, head and neck cancer, bladder cancer, endometrial cancer, esophageal cancer and prostate cancer in a subject, wherein the antibody-drug conjugate comprises an anti-TF antibody or an antigen-binding fragment thereof conjugated to a monomethyl auristatin or a functional analog thereof or a functional derivative thereof. In some embodiments, the cancer is colorectal cancer. In some embodiments, the cancer is non-small cell lung cancer. In some embodiments, the cancer is pancreatic cancer. In some embodiments, the cancer is head and neck cancer. In some embodiments, the cancer is bladder cancer. In some embodiments, the cancer is endometrial cancer. In some embodiments, the cancer is esophageal cancer. In some embodiments, the cancer is prostate cancer. In some embodiments, the colorectal cancer, non-small cell lung cancer, pancreatic cancer, head and neck cancer, bladder cancer, endometrial cancer, esophageal cancer or prostate cancer is a metastatic cancer. In some embodiments, the subject has relapsed, recurrent and/or metastatic colorectal cancer, non-small cell lung cancer, pancreatic cancer, head and neck cancer, bladder cancer, endometrial cancer, esophageal cancer or prostate cancer.

A. Anti-TF Antibody

Generally, anti-TF antibodies of the disclosure bind TF, e.g., human TF, and exert cytostatic and cytotoxic effects on malignant cells, such as colorectal cancer, non-small cell lung cancer, pancreatic cancer, head and neck cancer, bladder cancer, endometrial cancer, esophageal cancer and prostate cancer cells. Anti-TF antibodies of the disclosure are preferably monoclonal, and may be multispecific, human, humanized or chimeric antibodies, single chain antibodies, Fab fragments, F(ab′) fragments, fragments produced by a Fab expression library, and TF binding fragments of any of the above. In some embodiments, the anti-TF antibodies of the disclosure specifically bind TF. The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the anti-TF antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-TF antibodies or antigen-binding fragments thereof are human, murine (e.g., mouse and rat), donkey, sheep, rabbit, goat, guinea pig, camelid, horse, or chicken.

The anti-TF antibodies of the present disclosure may be monospecific, bispecific, trispecific or of greater multi specificity. Multispecific antibodies may be specific for different epitopes of TF or may be specific for both TF as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Anti-TF antibodies of the present disclosure may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a “CDR” or “complementary determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or MGT method.

CDR sequences provided herein are according to the MGT numbering scheme as described in Lefranc, M. P. et al., Dev. Comp. Immunol., 2003, 27, 55-77.

In certain embodiments antibodies of the disclosure comprise one or more CDRs of the antibody 011. See WO 2011/157741 and WO 2010/066803. The disclosure encompasses an antibody or derivative thereof comprising a heavy or light chain variable domain, said variable domain comprising (a) a set of three CDRs, in which said set of CDRs are from monoclonal antibody 011, and (b) a set of four framework regions, in which said set of framework regions differs from the set of framework regions in monoclonal antibody 011, and in which said antibody or derivative thereof binds to TF. In some embodiments, said antibody or derivative thereof specifically binds to TF. In certain embodiments, the anti-TF antibody is 011. The antibody 011 is also known as tisotumab.

In one aspect, anti-TF antibodies that compete with tisotumab binding to TF are also provided herein. Anti-TF antibodies that bind to the same epitope as tisotumab are also provided herein.

In one aspect, provided herein is an anti-TF antibody comprising 1, 2, 3, 4, 5, or 6 of the CDR sequences of tisotumab.

In one aspect, provided herein is an anti-TF antibody comprising a heavy chain variable region and a light chain variable region, wherein the heavy chain variable region comprises (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3; and/or wherein the light chain variable region comprises (i) CDR-L1 comprising the amino acid sequence of SEQ ID NO:4, (ii) CDR-L2 comprising the amino acid sequence of SEQ ID NO:5, and (iii) CDR-L3 comprising the amino acid sequence of SEQ ID NO:6.

An anti-TF antibody described herein may comprise any suitable framework variable domain sequence, provided that the antibody retains the ability to bind TF (e.g., human TF). As used herein, heavy chain framework regions are designated “HC-FR1-FR4,” and light chain framework regions are designated “LC-FR1-FR4.” In some embodiments, the anti-TF antibody comprises a heavy chain variable domain framework sequence of SEQ ID NO:9, 10, 11, and 12 (HC-FR1, HC-FR2, HC-FR3, and HC-FR4, respectively). In some embodiments, the anti-TF antibody comprises a light chain variable domain framework sequence of SEQ ID NO:13, 14, 15, and 16 (LC-FR1, LC-FR2, LC-FR3, and LC-FR4, respectively).

In some embodiments of the anti-TF antibodies described herein, the heavy chain variable domain comprises the amino acid sequence of

(SEQ ID NO: 7)
EVQLLESGGGLVQPGGSLRLSCAASGFTFSNYAMSWVRQAPGKGLEWVSS
ISGSGDYTYYTDSVKGRFTISRDNSKNTLYLQMNSLRAEDTAVYYCARSP
WGYYLDSWGQGTLVTVSS
and
the light chain variable domain comprises the
amino acid sequence of
(SEQ ID NO: 8)
DIQMTQSPPSLSASAGDRVTITCRASQGISSRLAWYQQKPEKAPKSLIYA
ASSLQSGVPSRFSGSGSGTDFTLTISSLQPEDFATYYCQQYNSYPYTFGQ
GTKLEIK.

In some embodiments of the anti-TF antibodies described herein, the heavy chain CDR sequences comprise the following:

a) CDR-H1
(GFTFSNYA (SEQ ID NO: 1));
b) CDR-H2
(ISGSGDYT (SEQ ID NO: 2));
and
c) CDR-H3
(ARSPWGYYLDS (SEQ ID NO: 3)).

In some embodiments of the anti-TF antibodies described herein, the heavy chain FR sequences comprise the following:

a) HC-FR1
(EVQLLESGGGLVQPGGSLRLSCAAS (SEQ ID NO: 9));
b) HC-FR2
(MSWVRQAPGKGLEWVSS (SEQ ID NO: 10));
c) HC-FR3
(YYTDSVKGRFTISRDNSKNTLYLQMNSLRAEDTAVYYC (SEQ ID
NO: 11));
and
d) HC-FR4
(WGQGTLVTVSS (SEQ ID NO: 12)).

In some embodiments of the anti-TF antibodies described herein, the light chain CDR sequences comprise the following:

a) CDR-L1
(QGISSR (SEQ ID NO: 4));
b) CDR-L2
(AAS (SEQ ID NO: 5));
and
c) CDR-L3
(QQYNSYPYT (SEQ ID NO: 6)).

In some embodiments of the anti-TF antibodies described herein, the light chain FR sequences comprise the following:

a) LC-FR1
(DIQMTQSPPSLSASAGDRVTITCRAS (SEQ ID NO: 13));
b) LC-FR2
(LAWYQQKPEKAPKSLIY (SEQ ID NO: 14));
c) LC-FR3
(SLQSGVPSRFSGSGSGTDFTLTISSLQPEDFATYYC (SEQ ID
NO: 15));
and
d) LC-FR4
(FGQGTKLEIK (SEQ ID NO: 16)).

In some embodiments, provided herein is an anti-TF antibody that binds to TF (e.g., human TF), wherein the antibody comprises a heavy chain variable region and a light chain variable region, wherein the antibody comprises:

(a) heavy chain variable domain comprising:
(1) an HC-FR1 comprising the amino acid sequence of SEQ ID NO:9;
(2) an CDR-H1 comprising the amino acid sequence of SEQ ID NO:1;
(3) an HC-FR2 comprising the amino acid sequence of SEQ ID NO:10;
(4) an CDR-H2 comprising the amino acid sequence of SEQ ID NO:2;
(5) an HC-FR3 comprising the amino acid sequence of SEQ ID NO:11;
(6) an CDR-H3 comprising the amino acid sequence of SEQ ID NO:3; and
(7) an HC-FR4 comprising the amino acid sequence of SEQ ID NO:12, and/or

(b) a light chain variable domain comprising:
(1) an LC-FR1 comprising the amino acid sequence of SEQ ID NO:13;
(2) an CDR-L1 comprising the amino acid sequence of SEQ ID NO4;
(3) an LC-FR2 comprising the amino acid sequence of SEQ ID NO:14;
(4) an CDR-L2 comprising the amino acid sequence of SEQ ID NO:5;
(5) an LC-FR3 comprising the amino acid sequence of SEQ ID NO:15;
(6) an CDR-L3 comprising the amino acid sequence of SEQ ID NO:6; and
(7) an LC-FR4 comprising the amino acid sequence of SEQ ID NO:16.

In one aspect, provided herein is an anti-TF antibody comprising a heavy chain variable domain comprising the amino acid sequence of SEQ ID NO:7 or comprising a light chain variable domain comprising the amino acid sequence of SEQ ID NO:8. In one aspect, provided herein is an anti-TF antibody comprising a heavy chain variable domain comprising the amino acid sequence of SEQ ID NO:7 and comprising a light chain variable domain comprising the amino acid sequence of SEQ ID NO:8.

In some embodiments, provided herein is an anti-TF antibody comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7. In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7 contains substitutions (e.g., conservative substitutions), insertions, or deletions relative to the reference sequence and retains the ability to bind to a TF (e.g., human TF). In certain embodiments, a total of 1 to 10 amino acids have been substituted, inserted and/or deleted in SEQ ID NO:7. In certain embodiments, substitutions, insertions, or deletions (e.g., 1, 2, 3, 4, or 5 amino acids) occur in regions outside the CDRs (i.e., in the FRs). In some embodiments, the anti-TF antibody comprises a heavy chain variable domain sequence of SEQ ID NO:7 including post-translational modifications of that sequence. In a particular embodiment, the heavy chain variable domain comprises one, two or three CDRs selected from: (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3.

In some embodiments, provided herein is an anti-TF antibody comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8. In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8 contains substitutions (e.g., conservative substitutions), insertions, or deletions relative to the reference sequence and retains the ability to bind to a TF (e.g., human TF). In certain embodiments, a total of 1 to 10 amino acids have been substituted, inserted and/or deleted in SEQ ID NO:8. In certain embodiments, substitutions, insertions, or deletions (e.g., 1, 2, 3, 4, or 5 amino acids) occur in regions outside the CDRs (i.e., in the FRs). In some embodiments, the anti-TF antibody comprises a light chain variable domain sequence of SEQ ID NO:8 including post-translational modifications of that sequence. In a particular embodiment, the light chain variable domain comprises one, two or three CDRs selected from: (a) CDR-L1 comprising the amino acid sequence of SEQ ID NO:4, (b) CDR-L2 comprising the amino acid sequence of SEQ ID NO:5, and (c) CDR-L3 comprising the amino acid sequence of SEQ ID NO:6.

In some embodiments, the anti-TF antibody comprises a heavy chain variable domain as in any of the embodiments provided above, and a light chain variable domain as in any of the embodiments provided above. In one embodiment, the antibody comprises the heavy chain variable domain sequence of SEQ ID NO:7 and the light chain variable domain sequence of SEQ ID NO:8, including post-translational modifications of those sequences.

In some embodiments, the anti-TF antibody of the anti-TF antibody-drug conjugate comprises: i) a heavy chain CDR1 comprising the amino acid sequence of SEQ ID NO: 1, a heavy chain CDR2 comprising the amino acid sequence of SEQ ID NO: 2, a heavy chain CDR3 comprising the amino acid sequence of SEQ ID NO: 3; and ii) a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 6.

In some embodiments, the anti-TF antibody of the anti-TF antibody-drug conjugate comprises: i) an amino acid sequence having at least 85% sequence identity to a heavy chain variable region comprising the amino acid sequence of SEQ ID NO: 7, and ii) an amino acid sequence having at least 85% sequence identity to a light chain variable region comprising the amino acid sequence of SEQ ID NO: 8.

In some embodiments, the anti-TF antibody of the anti-TF antibody-drug conjugate is a monoclonal antibody.

In some embodiments, the anti-TF antibody of the anti-TF antibody-drug conjugate is tisotumab, which is also known as antibody 011 as described in WO 2011/157741 and WO 2010/066803.

Anti-TF antibodies of the present invention may also be described or specified in terms of their binding affinity to TF (e.g., human TF). Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

The antibodies also include derivatives that are modified, i.e., by the covalent attachment of any type of molecule to the antibody such that covalent attachment does not prevent the antibody from binding to TF or from exerting a cytostatic or cytotoxic effect on HD cells. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

B. Antibody-Drug Conjugate Structure

In some aspects, the anti-TF antibody-drug conjugates described herein comprise a linker between an anti-TF antibody or antigen-binding fragment thereof as described herein and a cytostatic or cytotoxic drug. In some embodiments the linker is a non-cleavable linker. In some embodiments the linker is a cleavable linker.

In some embodiments, the linker is a cleavable peptide linker comprising maleimido caproyl (MC), the dipeptide valine-citrulline (vc) and p-aminobenzylcarbamate (PAB). In some embodiments, the cleavable peptide linker has the formula: MC-vc-PAB-, wherein:

a) MC is:

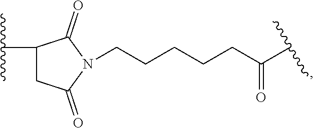 [SMILES: *C(=O)CCCCCN1C(=O)CC(*)C1=O]

b) vc is the dipeptide valine-citrulline, and

c) PAB is:

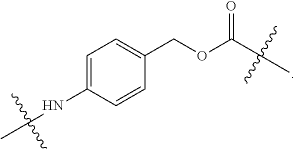 [SMILES: *Nc1ccc(COC(*)=O)cc1]

In some embodiments, the linker is a cleavable peptide linker comprising maleimido caproyl (MC). In some embodiments, the cleavable peptide linker has the formula: MC-, wherein:

a) MC is:

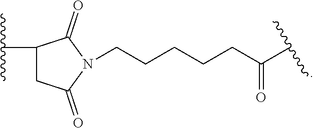 [SMILES: *C(=O)CCCCCN1C(=O)CC(*)C1=O]

In some embodiments, the linker is attached to sulphydryl residues of the anti-TF antibody or antigen-binding fragment thereof obtained by partial or full reduction of the anti-TF antibody or antigen-binding fragment thereof. In some embodiments, the linker is attached to sulphydryl residues of the anti-TF antibody or antigen-binding fragment thereof obtained by partial reduction of the anti-TF antibody or antigen-binding fragment thereof. In some embodiments, the linker is attached to sulphydryl residues of the anti-TF antibody or antigen-binding fragment thereof obtained by full reduction of the anti-TF antibody or antigen-binding fragment thereof.

In some aspects, the anti-TF antibody-drug conjugates described herein comprise a linker as described herein between an anti-TF antibody or antigen-binding fragment thereof as described herein and a cytostatic or cytotoxic drug. Auristatins have been shown to interfere with microtubule dynamics, GTP hydrolysis and nuclear and cellular division (See Woyke et al (2001) Antimicrob. Agents and Chemother. 45(12): 3580-3584) and have anti-cancer (See U.S. Pat. No. 5,663,149) and antifungal activity (See Pettit et al., (1998) Antimicrob. Agents and Chemother. 42: 2961-2965. For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023. In some embodiments of the anti-TF antibody-drug conjugates described herein, the cytostatic or cytotoxic drug is an auristatin or a functional analog thereof (e.g., functional peptide thereof) or a functional derivative thereof. In some embodiments, the auristatin is a monomethyl auristatin or a functional analog thereof (e.g., functional peptide thereof) or a functional derivative thereof.

In one embodiment, the auristatin is monomethyl auristatin E (MMAE):

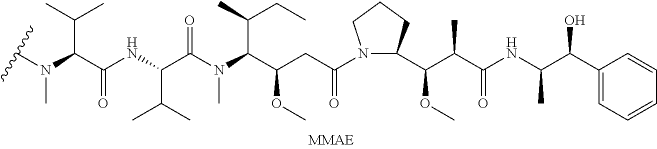 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein the wavy line indicates the attachment site for the linker.

In one embodiment, the auristatin is monomethyl auristatin F (MMAF):

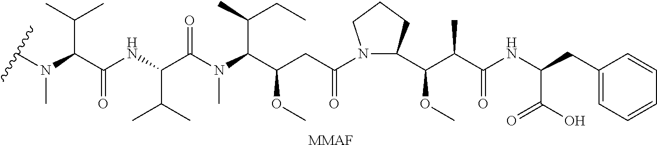

wherein the wavy line indicates the attachment site for the linker.

In one embodiment, the cleavable peptide linker has the formula: MC-vc-PAB-, and is attached to MMAE. The resulting linker-auristatin, MC-vc-PAB-MMAE is also designated vcMMAE. The vcMMAE drug linker moiety and conjugation methods are disclosed in WO2004010957, U.S. Pat. Nos. 7,659,241, 7,829,531 and 7,851,437. When vcMMAE is attached to an anti-TF antibody or antigen-binding fragment thereof as described herein, the resulting structure is:

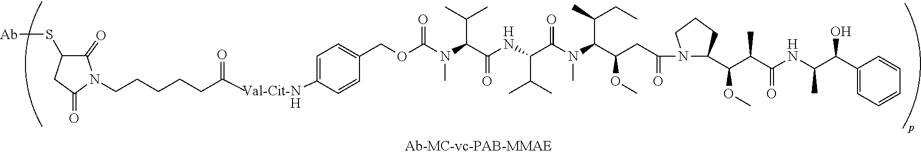

wherein p denotes a number from 1 to 8, e.g., 1, 2, 3, 4, 5, 6, 7 or 8, e.g., p may be from 3-5, S represents a sulphydryl residue of the anti-TF antibody and Ab designates an anti-TF antibody or antigen-binding fragment thereof as described herein. In one embodiment, the average value of p in a population of antibody-drug conjugates is about 4. In some embodiments, p is measured by hydrophobic interaction chromatography (HIC), for example by resolving drug-loaded species based on the increasing hydrophobicity with the least hydrophobic, unconjugated form eluting first and the most hydrophobic, 8-drug form eluting last with the area percentage of a peak representing the relative distribution of the particular drug-loaded antibody-drug conjugate species. See Ouyang, J., 2013, Antibody-Drug Conjugates, Methods in Molecular Biology (Methods and Protocols). In some embodiments, p is measured by reversed phase high-performance liquid chromatography (RP-HPLC), for example by first performing a reduction reaction to completely dissociate the heavy and light chains of the ADC, then separating the light and heavy chains and their corresponding drug-loaded forms on an RP column, where the percentage peak are from integration of the light chain and heavy chain peaks, combined with the assigned drug load for each peak, is used to calculate the weighted average drug to antibody ration. See Ouyang, J., 2013, Antibody-Drug Conjugates, Methods in Molecular Biology (Methods and Protocols).

In one embodiment, the cleavable peptide linker has the formula: MC-vc-PAB-, and is attached to MMAF. The resulting linker-auristatin, MC-vc-PAB-MMAF is also designated vcMMAF. In another embodiment, a non-cleavable linker MC is attached to MMAF. The resulting linker-auristatin MC-MMAF is also designated mcMMAF. Both the vcMMAF and mcMMAF drug linker moieties and conjugation methods are disclosed in WO2005081711 and U.S. Pat. No. 7,498,298. When vcMMAF or mcMMAF is attached to an anti-TF antibody or antigen-binding fragment thereof as described herein, the resulting structure is:

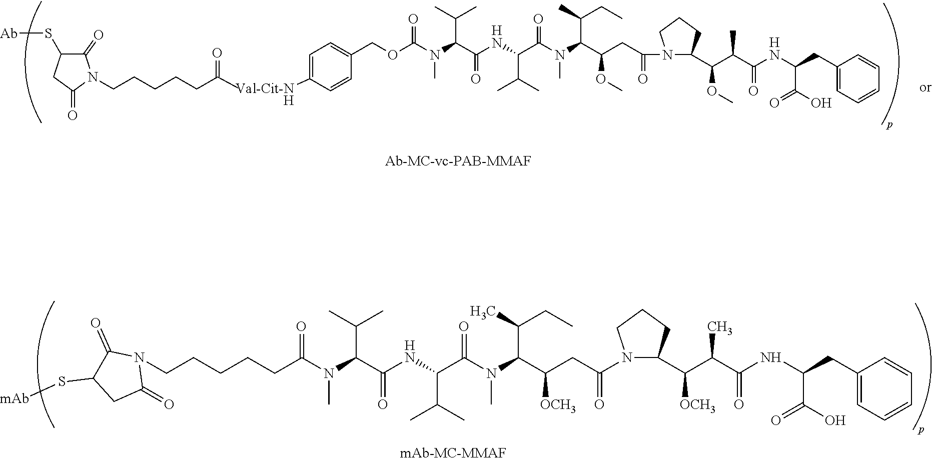

wherein p denotes a number from 1 to 8, e.g., 1, 2, 3, 4, 5, 6, 7 or 8, e.g., p may be from 3-5, S represents a sulphydryl residue of the anti-TF antibody and Ab or mAb designates an anti-TF antibody or antigen-binding fragment thereof as described herein. In one embodiment, the average value of p in a population of antibody-drug conjugates is about 4. In some embodiments, p is measured by hydrophobic interaction chromatography (HIC), for example by resolving drug-loaded species based on the increasing hydrophobicity with the least hydrophobic, unconjugated form eluting first and the most hydrophobic, 8-drug form eluting last with the area percentage of a peak representing the relative distribution of the particular drug-loaded antibody-drug conjugate species. See Ouyang, J., 2013, Antibody-Drug Conjugates, Methods in Molecular Biology (Methods and Protocols). In some embodiments, p is measured by reversed phase high-performance liquid chromatography (RP-HPLC), for example by first performing a reduction reaction to completely dissociate the heavy and light chains of the ADC, then separating the light and heavy chains and their corresponding drug-loaded forms on an RP column, where the percentage peak are from integration of the light chain and heavy chain peaks, combined with the assigned drug load for each peak, is used to calculate the weighted average drug to antibody ration. See Ouyang, J., 2013, Antibody-Drug Conjugates, Methods in Molecular Biology (Methods and Protocols).

In one embodiment, the antibody-drug conjugate is tisotumab vedotin.

C. Nucleic Acids, Host Cells and Methods of Production

In some aspects, also provided herein are nucleic acids encoding an anti-TF antibody or antigen-binding fragment thereof as described herein. Further provided herein are vectors comprising the nucleic acids encoding an anti-TF antibody or antigen-binding fragment thereof as described herein. Further provided herein are host cells expressing the nucleic acids encoding an anti-TF antibody or antigen-binding fragment thereof as described herein. Further provided herein are host cells comprising the vectors comprising the nucleic acids encoding an anti-TF antibody or antigen-binding fragment thereof as described herein. Methods of producing an anti-TF antibody, linker and anti-TF antibody-drug conjugate are described in U.S. Pat. No. 9,168,314.

The anti-TF antibodies described herein may be prepared by well-known recombinant techniques using well known expression vector systems and host cells. In one embodiment, the antibodies are prepared in a CHO cell using the GS expression vector system as disclosed in De la Cruz Edmunds et al., 2006, Molecular Biotechnology 34; 179-190, EP216846, U.S. Pat. No. 5,981,216, WO 87/04462, EP323997, U.S. Pat. Nos. 5,591,639, 5,658,759, EP338841, U.S. Pat. Nos. 5,879,936, and 5,891,693.

After isolating and purifying the anti-TF antibodies from the cell media using well known techniques in the art, they are conjugated with an auristatin via a linker as described in U.S. Pat. No. 9,168,314.

Monoclonal anti-TF antibodies described herein may e.g. be produced by the hybridoma method first described by Kohler et al., Nature, 256, 495 (1975), or may be produced by recombinant DNA methods. Monoclonal antibodies may also be isolated from phage antibody libraries using the techniques described in, for example, Clackson et al., Nature, 352, 624-628 (1991) and Marks et al., JMol, Biol., 222(3):581-597 (1991). Monoclonal antibodies may be obtained from any suitable source. Thus, for example, monoclonal antibodies may be obtained from hybridomas prepared from murine splenic B cells obtained from mice immunized with an antigen of interest, for instance in form of cells expressing the antigen on the surface, or a nucleic acid encoding an antigen of interest. Monoclonal antibodies may also be obtained from hybridomas derived from antibody-expressing cells of immunized humans or non-human mammals such as rats, dogs, primates, etc.

In one embodiment, the antibody (e.g., anti-TF antibody) of the invention is a human antibody. Human monoclonal antibodies directed against TF may be generated using transgenic or transchromosomal mice carrying parts of the human immune system rather than the mouse system. Such transgenic and transchromosomic mice include mice referred to herein as HuMAb mice and KM mice, respectively, and are collectively referred to herein as “transgenic mice”.

The HuMAb mouse contains a human immunoglobulin gene minilocus that encodes unrearranged human heavy (μ and γ) and κ light chain immunoglobulin sequences, together with targeted mutations that inactivate the endogenous μ and κ chain loci (Lonberg, N. et al., Nature, 368, 856-859 (1994)). Accordingly, the mice exhibit reduced expression of mouse IgM or κ and in response to immunization, the introduced human heavy and light chain transgenes undergo class switching and somatic mutation to generate high affinity human IgG,κ monoclonal antibodies (Lonberg, N. et al. (1994), supra; reviewed in Lonberg, N. Handbook ofExperimentalPharmacology 113, 49-101 (1994), Lonberg, N. and Huszar. D., Intern. Rev. Immunol, Vol. 13 65-93 (1995) and Harding, F. and Lonberg, N. Ann, N.Y. Acad. Sci 764:536-546 (1995)). The preparation of HuMAb mice is described in detail in Taylor, L. et al., Nucleic Acids Research. 20:6287-6295 (1992), Chen, J. et al., International Immunology. 5:647-656 (1993), Tuaillon at al., J. Immunol, 152:2912-2920 (1994), Taylor, L. et al., International Immunology, 6:579-591 (1994), Fishwild, D. et al., Nature Biotechnology, 14:845-851 (1996). See also U.S. Pat. Nos. 5,545,806, 5,569,825, 5,625,126, 5,633,425, 5,789,650, 5,877,397, 5,661,016, 5,814,318, 5,874,299, 5,770,429, 5,545,807, WO 98/24884, WO 94/25585, WO 93/1227, WO 92/22645, WO 92/03918 and WO 01/09187.

The HCo7 mice have a JKD disruption in their endogenous light chain (kappa) genes (as described in Chen et al, EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo7 human heavy chain transgene (as described in U.S. Pat. No. 5,770,429).

The HCo12 mice have a JKD disruption in their endogenous light chain (kappa) genes (as described in Chen et al., EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo12 human heavy chain transgene (as described in Example 2 of WO 01/14424).

The HCo17 transgenic mouse strain (see also US 2010/0077497) was generated by coinjection of the 80 kb insert of pHC2 (Taylor et al. (1994) Int. Immunol., 6:579-591), the Kb insert of pVX6, and a−460 kb yeast artificial chromosome fragment of the yIgH24 chromosome. This line was designated (HCo17) 25950. The (HCol7) 25950 line was then bred with mice comprising the CMD mutation (described in Example 1 of PCT Publication WO 01109187), the JKD mutation (Chen et al, (1993) EMBO J. 12:811-820), and the (KC05) 9272 transgene (Fishwild et al. (1996) Nature Biotechnology, 14:845-851). The resulting mice express human immunoglobulin heavy and kappa light chain transgenes in a background homozygous for disruption of the endogenous mouse heavy and kappa light chain loci.

The HCo20 transgenic mouse strain is the result of a co-injection of minilocus 30 heavy chain transgene pHC2, the germline variable region (Vh)-containing YAC yIgH10, and the minilocus construct pVx6 (described in WO09097006). The (HCo20) line was then bred with mice comprising the CMD mutation (described in Example 1 of PCT Publication WO 01/09187), the JKD mutation (Chen et al. (1993) EMBO J. 12:811-820), and the (KC05) 9272 trans gene (Fishwild eta). (1996) Nature Biotechnology, 14:845-851). The resulting mice express human 10 immunoglobulin heavy and kappa light chain transgenes in a background homozygous for disruption of the endogenous mouse heavy and kappa light chain loci.

In order to generate HuMab mice with the salutary effects of the Balb/c strain, HuMab mice were crossed with KC005 [MIK] (Balb) mice which were generated by backcrossing the KC05 strain (as described in Fishwild et al. (1996) Nature Biotechnology, 14:845-851) to wild-type Balb/c mice to generate mice as described in WO09097006. Using this crossing Balb/c hybrids were created for HCol2, HCo17, and HCo20 strains.

In the KM mouse strain, the endogenous mouse kappa light chain gene has been homozygously disrupted as described in Chen et al., EMBO J. 12:811-820 (1993) and the endogenous mouse heavy chain gene has been homozygously disrupted as described in Example 1 of WO 01/09187, This mouse strain carries a human kappa light chain transgene, KCo5, as described in Fishwild et al., Nature Biotechnology, 14:845-851 (1996). This mouse strain also carries a human heavy chain transchromosome composed of chromosome 14 fragment hCF (SC20) as described in WO 02/43478.

Splenocytes from these transgenic mice may be used to generate hybridomas that secrete human monoclonal antibodies according to well-known techniques, Human monoclonal or polyclonal antibodies of the present invention, or antibodies of the present invention originating from other species may also be generated transgenically through the generation of another non-human mammal or plant that is transgenic for the immunoglobulin heavy and light chain sequences of interest and production of the antibody in a recoverable form therefrom. In connection with the transgenic production in mammals, antibodies may be produced in, and recovered from, the milk of goats, cows, or other mammals. See for instance U.S. Pat. Nos. 5,827,690, 5,756,687, 5,750,172 and 5,741,957.

Further, human antibodies of the present invention or antibodies of the present invention from other species may be generated through display-type technologies, including, without limitation, phage display, retroviral display, ribosomal display, and other techniques, using techniques well known in the art and the resulting molecules may be subjected to additional maturation, such as affinity maturation, as such techniques are well known in the art (See for instance Hoogenboom et al., J. Mol, Biol. 227(2):381-388 (1992) (phage display), Vaughan et al., Nature Biotech, 14:309 (1996) (phage display), Hanes and Plucthau, PNAS USA 94:4937-4942 (1997) (ribosomal display), Parmley and Smith, Gene, 73:305-318 (1988) (phage display), Scott, TIBS. 17:241-245 (1992), Cwirla et al., PNAS USA, 87:6378-6382 (1990), Russel et al., Nucl. Acids Research, 21:1081-4085 (1993), Hogenboom et al., Immunol, Reviews, 130:43-68 (1992), Chiswell and McCafferty, TIBTECH, 10:80-84 (1992), and U.S. Pat. No. 5,733,743). If display technologies are utilized to produce antibodies that are not human, such antibodies may be humanized.

### III. Methods of Treatment

The invention provides methods for treating cancer in a subject with an anti-TF antibody-drug conjugate described herein, wherein the cancer is colorectal cancer, non-small cell lung cancer, pancreatic cancer, head and neck cancer, bladder cancer, endometrial cancer, esophageal cancer or prostate cancer. In one aspect, the antibody-drug conjugate is tisotumab vedotin. In a particular embodiment, the subject is a human.

In another aspect the present invention provides an antibody-drug conjugate that binds to TF for use in the treatment of cancer wherein the antibody-drug conjugate comprises an anti-TF antibody or an antigen-binding fragment thereof conjugated to a monomethyl auristatin or a functional analog thereof or a functional derivative thereof and wherein the cancer is colorectal cancer, non-small cell lung cancer, pancreatic cancer, head and neck cancer, bladder cancer, endometrial cancer, esophageal cancer or prostate cancer. In one aspect, the antibody-drug conjugate is tisotumab vedotin. In a particular embodiment, the subject is a human.

In some embodiments, the subject has been previously treated for the colorectal cancer, non-small cell lung cancer, pancreatic cancer, head and neck cancer, bladder cancer, endometrial cancer, esophageal cancer or prostate cancer. In some embodiments, the subject did not respond to the treatment (e.g., the subject experienced disease progression during treatment). In some embodiments, the subject relapsed after the treatment. In some embodiments, the subject experienced disease progression after the treatment. In some embodiments, the treatment previously administered to the subject was not an anti-TF antibody-drug conjugate as described herein.

A. Colorectal Cancer

Colorectal cancer is the third leading cause of cancer-related deaths in men and women in the United States. Though colorectal cancer mortality rates have steadily declined in recent years (dropping an estimated 4% per year between 2008 and 2011) due in part to better screening rates for early detection, 5 year survival for patients with metastatic colorectal cancer is only 21%.

Improvements have been made in systemic therapy for non-operable colorectal cancer since the days that fluorouracil was the sole active agent, but clinical trials are still recommended for patients when conventional therapies or combinations have failed. Although systemic therapies have produced meaningful improvements in OS, PFS, and response rate for patients with colorectal cancer, this benefit is most pronounced with regimens containing irinotecan, oxaliplatin in combination with 5-FU, and biologics. Recently, immunotherapy—pembrolizumab and nivolumab—has emerged as a new option for treatment of patients who have tumors with a high level of microsatellite instability (MSI-H) or who are deficient in DNA mismatch repair enzymes, but only 3.5 to 6.5% of stage IV colorectal cancers are in this subgroup.

The approach to subsequent therapy is variable and may include maintenance chemotherapy or a switch to a different regimen altogether because of disease progression or intolerance to the initial regimen. For patients with metastatic colorectal cancer, the model of distinct “lines” of chemotherapy (in which regimens containing non-cross-resistant drugs are each used in succession until disease progression) is being abandoned in favor of a “continuum of care” approach (Goldberg R M et al., 2007, Oncologist 12(1): 38-50).

The invention provides methods for treating colorectal cancer in a subject with an antibody-drug conjugate described herein. In one aspect, the antibody-drug conjugates described herein are for use in a method of treating colorectal cancer in a subject. In one aspect, the antibody-drug conjugate is tisotumab vedotin. In some embodiments, the subject has not been previously treated for the colorectal cancer. In some embodiments, the subject has received at least one previous treatment for the colorectal cancer. In some embodiments, the subject received prior systemic therapy for the colorectal cancer. In some embodiments, the subject experienced disease progression on or after the systemic therapy. In some embodiments, the subject received no more than 3 rounds of prior systemic therapy. In some embodiments, the subject received 1, 2 or 3 rounds of prior systemic therapy. In some embodiments, the subject received 1 round of prior systemic therapy. In some embodiments, the subject received 2 rounds of prior systemic therapy. In some embodiments, the subject received 3 rounds of prior systemic therapy. In some embodiments, the colorectal cancer is non-operable. In some embodiments, the subject has been previously treated with one or more agents selected from the group consisting of fluoropyrimidine, oxaliplatin, irinotecan, bevacizumab, cetuximab, panitumab and a checkpoint inhibitor. In some embodiments, the subject has been previously treated with one or more agents selected from the group consisting of fluoropyrimidine, oxaliplatin, irinotecan and bevacizumab. In some embodiments, the subject has been previously treated with fluoropyrimidine. In some embodiments, the subject has been previously treated with oxaliplatin. In some embodiments, the subject has been previously treated with irinotecan. In some embodiments, the subject has been previously treated with bevacizumab. In some embodiments, the subject has been previously treated with one or more agents selected from the group consisting of cetuximab, panitumab and a checkpoint inhibitor. In some embodiments, the subject has been previously treated with cetuximab. In some embodiments, the subject has been previously treated with panitumab. In some embodiments, the subject has been previously treated with a checkpoint inhibitor. In some embodiments, the checkpoint inhibitor is an inhibitor of PD-1, PD-L1, CTLA-4, PD-L2, LAG3, Tim3, 2B4, A2aR, ID02, B7-H3, B7-H4, BTLA, CD2, CD20, CD27, CD28, CD30, CD33, CD40, CD52, CD70, CD80, CD86, CD112, CD137, CD 160, CD226, CD276, DR3, OX-40, GAL9, GITR, ICOS, HVEM, IDOI, KIR, LAIR, LIGHT, MARCO, PS, SLAM, TIGIT, VISTA, and/or VTCN1. In some embodiments, the checkpoint inhibitor is an inhibitor of PD-1, PD-L1 and/or CTLA-4. In some embodiments, the checkpoint inhibitor is an inhibitor of PD-1. In some embodiments, the checkpoint inhibitor is selected from the group consisting of nivolumab (OPDIVO®, BMS-936558, MDX-1106 or MK-34775), pembrolizumab (KEYTRUDA®, MK-3475), pidilizumab (CT-011) and cemiplimab (REGN2810). In some embodiments, the checkpoint inhibitor is an inhibitor of PD-L1. In some embodiments, the checkpoint inhibitor is selected from the group consisting of atezolizumab (TECENTRIQ®, MPDL3280A), avelumab (BAVENCIO®), durvalumab and BMS-936559. In some embodiments, the checkpoint inhibitor is an inhibitor of CTLA-4. In some embodiments, the checkpoint inhibitor is selected from the group consisting of ipilimumab and tremelimumab. In some embodiments, the colorectal cancer is an advanced stage cancer. In some embodiments, the advanced stage cancer is a stage 3 or 4 cancer. In some embodiments, the advanced stage cancer is a metastatic cancer. In some embodiments, the colorectal cancer is a recurrent cancer. In some embodiments, the subject received prior treatment with standard of care therapy for the cancer and failed the prior treatment. Ina particular embodiment, the subject is a human.

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the colorectal cancer cells from the subject express TF. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the colorectal cancer cells from the subject express TF. In some embodiments, the percentage of cells that express TF is determined using immunohistochemistry (IHC). In some embodiments, the percentage of cells that express TF is determined using flow cytometry. In some embodiments, the percentage of cells that express TF is determined using an enzyme-linked immunosorbent assay (ELISA).

B. Non-Small Cell Lung Cancer

Lung cancer remains the leading cause of death from cancer in the United States. Treatments with curative intent for patients with early stage disease include surgery, chemotherapy, radiation therapy, or a combined modality approach. Lung cancer typically undergoes epithelial-mesenchymal transition with early metastatic spread. The symptoms are often difficult for patients to recognize in the early stages of disease. Because of these two factors, a majority of patients are diagnosed with advanced stage disease, which is usually incurable.

NSCLC represents up to 80% of all lung cancers. Within the subtypes of NSCLC, squamous cell carcinoma (SCC/NSCLC) represents approximately 30% of NSCLC. Systemic therapy can significantly prolong survival and help maintain quality of life in patients who present with stage IV squamous NSCLC or who develop advanced disease following their initial definitive therapy. Histology provides insight into the optimal agents to combine with a platinum compound and molecular characterization of the tumor. Patients with SCC/NSCLC should have tumor assessed for the expression of programmed death ligand-1 (PD-L1). The choice of initial therapy is guided by this information. For patients with SCC/NSCLC whose tumors do not express high levels of PD-L1, the preferred first line option is a platinum-based chemotherapy doublet that does not contain pemetrexed or anti-VEGF. Other platinum partners that may be used in initial therapy for SCC/NSCLC include necitumumab, a monoclonal antibody that targets EGFR, e.g., in combination with gemcitabine and cisplatin. For patients with at least 50% tumor cell staining for PD-L1 and without contraindications to immunotherapy, first-line treatment with the anti-PD-1 inhibitor pembrolizumab should be offered. Pembrolizumab should be continued until progression or intolerable toxicity occurs.

Following disease progression from first line treatment, multiple factors need to be considered, including the type of prior treatment, PD-L1 expression, and performance status. Systemic therapy trials for second line and later metastatic NSCLC include docetaxel, vinorelbine or ifosfamide, OPDIVO®, docetaxel, KEYTRUDA®, and TECENTRIQ®. The most preferred treatment regimen for SCC/NSCLC patients who progress on an initial combination chemotherapy regimen is immunotherapy with an anti-PD-1 or PD-L1 antibody. Combination chemotherapy should be considered for patients whose disease has progressed after receiving PD-1/L1 inhibitors.

The invention provides methods for treating non-small cell lung cancer with an antibody-drug conjugate described herein. In one aspect, the antibody-drug conjugates described herein are for use in a method of treating non-small cell lung cancer in a subject. In one aspect, the antibody-drug conjugate is tisotumab vedotin. In some embodiments, the subject has not been previously treated for the non-small cell lung cancer. In some embodiments, the subject has received at least one previous treatment for the non-small cell lung cancer. In some embodiments, the subject received prior systemic therapy for the non-small cell lung cancer. In some embodiments, the subject experienced disease progression on or after the systemic therapy. In some embodiments, the subject received no more than 2 rounds of prior systemic therapy. In some embodiments, the subject received 1 or 2 rounds of prior systemic therapy. In some embodiments, the subject received 1 round of prior systemic therapy. In some embodiments, the subject received 2 rounds of prior systemic therapy. In some embodiments, the subject has been previously treated with one or more agents selected from the group consisting of a platinum-based therapy and a checkpoint inhibitor. In some embodiments, the subject has been previously treated with a platinum-based therapy. In some embodiments, the platinum-based therapy is selected from the group consisting of carboplatin, cisplatin, oxaliplatin, nedaplatin, triplatin tetranitrate, phenanthriplatin, picoplatin and satraplatin. In some embodiments, the platinum-based therapy is carboplatin. In some embodiments, the platinum-based therapy is cisplatin. In some embodiments, the platinum-based therapy is oxaliplatin. In some embodiments, the platinum-based therapy is nedaplatin. In some embodiments, the platinum-based therapy is triplatin tetranitrate. In some embodiments, the platinum-based therapy is phenanthriplatin. In some embodiments, the platinum-based therapy is picoplatin. In some embodiments, the platinum-based therapy is satraplatin. In some embodiments, the subject has been previously treated with a checkpoint inhibitor. In some embodiments, the checkpoint inhibitor is an inhibitor of PD-1, PD-L1, CTLA-4, PD-L2, LAG3, Tim3, 2B4, A2aR, ID02, B7-H3, B7-H4, BTLA, CD2, CD20, CD27, CD28, CD30, CD33, CD40, CD52, CD70, CD80, CD86, CD112, CD137, CD 160, CD226, CD276, DR3, OX-40, GAL9, GITR, ICOS, HVEM, IDOI, KIR, LAIR, LIGHT, MARCO, PS, SLAM, TIGIT, VISTA, and/or VTCN1. In some embodiments, the checkpoint inhibitor is an inhibitor of PD-1, PD-L1 and/or CTLA-4. In some embodiments, the checkpoint inhibitor is an inhibitor of PD-1. In some embodiments, the checkpoint inhibitor is selected from the group consisting of nivolumab (OPDIVO®, BMS-936558, MDX-1106 or MK-34775), pembrolizumab (KEYTRUDA®, MK-3475), pidilizumab (CT-011) and cemiplimab (REGN2810). In some embodiments, the checkpoint inhibitor is an inhibitor of PD-L1. In some embodiments, the checkpoint inhibitor is selected from the group consisting of atezolizumab (TECENTRIQ®, MPDL3280A), avelumab (BAVENCIO®), durvalumab and BMS-936559. In some embodiments, the checkpoint inhibitor is an inhibitor of CTLA-4. In some embodiments, the checkpoint inhibitor is selected from the group consisting of ipilimumab and tremelimumab. In some embodiments, the non-small cell lung cancer is squamous cell carcinoma. In some embodiments, the non-small cell lung cancer has predominant squamous histology. In some embodiments greater than 75%, greater than 80%, greater than 85%, greater than 90% or greater than 95% of the non-small cell lung cancer cells have squamous histology. In some embodiments greater than 75% of the non-small cell lung cancer cells have squamous histology. In some embodiments greater than 80% of the non-small cell lung cancer cells have squamous histology. In some embodiments greater than 85% of the non-small cell lung cancer cells have squamous histology. In some embodiments greater than 90% of the non-small cell lung cancer cells have squamous histology. In some embodiments greater than 95% of the non-small cell lung cancer cells have squamous histology. In some embodiments, the non-small cell lung cancer is adenocarcinoma. In some embodiments, the non-small cell lung cancer is an advanced stage cancer. In some embodiments, the advanced stage cancer is a stage 3 or 4 cancer. In some embodiments, the advanced stage cancer is a metastatic cancer. In some embodiments, the non-small cell lung cancer is a recurrent cancer. In some embodiments, the subject received prior treatment with standard of care therapy for the cancer and failed the prior treatment. Ina particular embodiment, the subject is a human.

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the non-small cell lung cancer cells from the subject express TF. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the non-small cell lung cancer cells from the subject express TF. In some embodiments, the percentage of cells that express TF is determined using immunohistochemistry (IHC). In some embodiments, the percentage of cells that express TF is determined using flow cytometry. In some embodiments, the percentage of cells that express TF is determined using an enzyme-linked immunosorbent assay (ELISA).

C. Pancreatic Cancer

Pancreatic cancer is the third leading cause of cancer-related death in the United States in 2016. Five year survival for people with metastatic pancreatic cancer remains a dismal 8% in the US and may be as low as 4% worldwide. Surgical resection offers the only chance of cure. However, only 15% to 20% of patients have resectable disease at initial diagnosis; the majority have either locally advanced or metastatic cancer. Metastatic pancreatic cancer patients have very few effective treatment options and are often treated only with palliative care. First line combination regimens including FOLFIRINOX or nab-paclitaxel plus gemcitabine are often options for patients with a reasonable performance status and have been shown to prolong OS by several months. Second line and later treatments offer limited efficacy with significant treatment-related toxicity. Preferred regimens in this group include liposomal irinotecan (ONIVYDE*) with 5-FU/leucovorin, FOLFOX, and gemcitabine in combination with nab-paclitaxel, erlotinib, or bevacizumab.

The invention provides methods for treating pancreatic cancer with an antibody-drug conjugate described herein. In one aspect, the antibody-drug conjugates described herein are for use in a method of treating pancreatic cancer in a subject. In one aspect, the antibody-drug conjugate is tisotumab vedotin. In some embodiments, the subject has not been previously treated for the pancreatic cancer. In some embodiments, the subject has received at least one previous treatment for the pancreatic cancer. In some embodiments, the subject received prior systemic therapy for the pancreatic cancer. In some embodiments, the subject experienced disease progression on or after the systemic therapy. In some embodiments, the subject received no more than 1 round of prior systemic therapy. In some embodiments, the subject received 1 round of prior systemic therapy. In some embodiments, the subject has been previously treated with one or more agents selected from the group consisting of gemcitabine and 5-fluorouracil (5-FU). In some embodiments, the subject has been previously treated with gemcitabine. In some embodiments, the subject has been previously treated with 5-fluorouracil. In some embodiments, the pancreatic cancer is not resectable. In some embodiments, the pancreatic cancer is exocrine pancreatic adenocarcinoma. In some embodiments, the pancreatic cancer has predominant adenocarcinoma histology. In some embodiments greater than 75%, greater than 80%, greater than 85%, greater than 90% or greater than 95% of the pancreatic cancer cells have adenocarcinoma histology. In some embodiments greater than 75% of the pancreatic cancer cells have adenocarcinoma histology. In some embodiments greater than 80% of the pancreatic cancer cells have adenocarcinoma histology. In some embodiments greater than 85% of the pancreatic cancer cells have adenocarcinoma histology. In some embodiments greater than 90% of the pancreatic cancer cells have adenocarcinoma histology. In some embodiments greater than 95% of the pancreatic cancer a cells have adenocarcinoma histology. In some embodiments, the pancreatic cancer is an advanced stage cancer. In some embodiments, the advanced stage cancer is a stage 3 or 4 cancer. In some embodiments, the advanced stage cancer is a metastatic cancer. In some embodiments, the pancreatic cancer is a recurrent cancer. In some embodiments, the subject received prior treatment with standard of care therapy for the cancer and failed the prior treatment. In a particular embodiment, the subject is a human.

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the pancreatic cancer cells from the subject express TF. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the pancreatic cancer cells from the subject express TF. In some embodiments, the percentage of cells that express TF is determined using immunohistochemistry (IHC). In some embodiments, the percentage of cells that express TF is determined using flow cytometry. In some embodiments, the percentage of cells that express TF is determined using an enzyme-linked immunosorbent assay (ELISA).

D. Head and Neck Cancer

Head and neck cancers account for approximately 4% of all cancers in the United States. More than 90-95% of oral and nasopharyngeal cancers are of squamous histology. Surgical resection, radiotherapy, and/or chemoradiation are frequently recommended for patients with early-stage or localized disease. Palliative chemotherapy, immunotherapy and/or supportive care are the most appropriate options for patients with locally recurrent or metastatic disease that are not amenable to definitive therapy. For patients with recurrent or de novo metastatic disease, primary treatment is with systemic therapy. Platinum-based regimens are the preferred standard of care in this setting. Cetuximab in combination with a platinum-5-FU regimen has demonstrated clinically meaningful benefit with an improvement in median OS of 10.1 months compared to 7.4 months for platinum/5-FU alone. For patients progressing on first line treatment, second line treatment is with single agent chemotherapy, targeted therapy, or a checkpoint inhibitor (CPI). Prolonged durations of response (DORs) have led the CPIs to be the preferred treatment in this setting. Both nivolumab and pembrolizumab received FDA approval for treatment in the second line setting in 2016. After failure of first-line chemotherapy, responses to second-line chemotherapy are uncommon, particularly when contemporary response criteria are applied, and there is no evidence that subsequent chemotherapy prolongs survival.

The invention provides methods for treating head and neck cancer with an antibody-drug conjugate described herein. In one aspect, the antibody-drug conjugates described herein are for use in a method of treating head and neck cancer in a subject. In one aspect, the antibody-drug conjugate is tisotumab vedotin. In some embodiments, the subject has not been previously treated for the head and neck cancer. In some embodiments, the head and neck cancer is squamous cell carcinoma. In some embodiments, the subject has received at least one previous treatment for the head and neck cancer. In some embodiments, the subject received prior systemic therapy for the head and neck cancer. In some embodiments, the subject experienced disease progression on or after the systemic therapy. In some embodiments, the subject received no more than 2 rounds of prior systemic therapy. In some embodiments, the subject received 1 or 2 rounds of prior systemic therapy. In some embodiments, the subject received 1 round of prior systemic therapy. In some embodiments, the subject received 2 rounds of prior systemic therapy. In some embodiments, the subject has been previously treated with one or more agents selected from the group consisting of a platinum-based therapy, a checkpoint inhibitor and an anti-epithelial growth factor receptor therapy. In some embodiments, the subject has been previously treated with one or more agents selected from the group consisting of a platinum-based therapy and a checkpoint inhibitor. In some embodiments, the subject has been previously treated with a platinum-based therapy. In some embodiments, the platinum-based therapy is selected from the group consisting of carboplatin, cisplatin, oxaliplatin, nedaplatin, triplatin tetranitrate, phenanthriplatin, picoplatin and satraplatin. In some embodiments, the platinum-based therapy is carboplatin. In some embodiments, the platinum-based therapy is cisplatin. In some embodiments, the platinum-based therapy is oxaliplatin. In some embodiments, the platinum-based therapy is nedaplatin. In some embodiments, the platinum-based therapy is triplatin tetranitrate. In some embodiments, the platinum-based therapy is phenanthriplatin. In some embodiments, the platinum-based therapy is picoplatin. In some embodiments, the platinum-based therapy is satraplatin. In some embodiments, the subject has been previously treated with a checkpoint inhibitor. In some embodiments, the checkpoint inhibitor is an inhibitor of PD-1, PD-L1, CTLA-4, PD-L2, LAG3, Tim3, 2B4, A2aR, ID02, B7-H3, B7-H4, BTLA, CD2, CD20, CD27, CD28, CD30, CD33, CD40, CD52, CD70, CD80, CD86, CD112, CD137, CD 160, CD226, CD276, DR3, OX-40, GAL9, GITR, ICOS, HVEM, IDOI, KIR, LAIR, LIGHT, MARCO, PS, SLAM, TIGIT, VISTA, and/or VTCN1. In some embodiments, the checkpoint inhibitor is an inhibitor of PD-1, PD-L1 and/or CTLA-4. In some embodiments, the checkpoint inhibitor is an inhibitor of PD-1. In some embodiments, the checkpoint inhibitor is selected from the group consisting of nivolumab (OPDIVO®, BMS-936558, MDX-1106 or MK-34775), pembrolizumab (KEYTRUDA®, MK-3475), pidilizumab (CT-011) and cemiplimab (REGN2810). In some embodiments, the checkpoint inhibitor is an inhibitor of PD-L1. In some embodiments, the checkpoint inhibitor is selected from the group consisting of atezolizumab (TECENTRIQ®, MPDL3280A), avelumab (BAVENCIO®), durvalumab and BMS-936559. In some embodiments, the checkpoint inhibitor is an inhibitor of CTLA-4. In some embodiments, the checkpoint inhibitor is selected from the group consisting of ipilimumab and tremelimumab. In some embodiments, the subject has been previously treated with an anti-epithelial growth factor receptor therapy. In some embodiments, the anti-epithelial growth factor receptor therapy is selected from the group consisting of gefitinib, erlotinib, afatinib, brigatinib, icotinib, lapatinib, osimertinib, cetuximab, panitumumab, zalutumumab, nimotuzumab and matuzumab. In some embodiments, the head and neck cancer is an advanced stage cancer. In some embodiments, the advanced stage cancer is a stage 3 or 4 cancer. In some embodiments, the advanced stage cancer is a metastatic cancer. In some embodiments, the head and neck cancer is a recurrent cancer. In some embodiments, the subject received prior treatment with standard of care therapy for the cancer and failed the prior treatment. Ina particular embodiment, the subject is a human.

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the head and neck cancer cells from the subject express TF. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the head and neck cancer cells from the subject express TF. In some embodiments, the percentage of cells that express TF is determined using immunohistochemistry (IHC). In some embodiments, the percentage of cells that express TF is determined using flow cytometry. In some embodiments, the percentage of cells that express TF is determined using an enzyme-linked immunosorbent assay (ELISA).

E. Bladder Cancer

Bladder cancer is the sixth most common cancer in the United States, with an estimated 76,960 new cases diagnosed in 2016. Of these patients, 16,390 deaths were estimated to have occurred, with men being more likely to be affected than women. The 5-year relative survival rate for all stages combined is 77%. However, survival rates depend on many factors, including the histology and stage of bladder cancer diagnosed. For patients with bladder cancer that is invasive but not yet spread outside the bladder, the 5-year survival rate is 70%. For patients with bladder cancer that extends through the bladder to the surrounding tissue and/or organs, the 5-year survival rate is 34%. A cisplatin-based chemotherapy regimen followed by surgical removal of the bladder or radiation therapy and concomitant chemotherapy is currently the standard treatment for patients with invasive bladder cancer. More effective treatments for bladder cancer, particularly for patients with advanced or metastatic bladder cancer, are urgently needed.

The invention provides methods for treating bladder cancer with an antibody-drug conjugate described herein. In one aspect, the antibody-drug conjugates described herein are for use in a method of treating bladder cancer in a subject. In one aspect, the antibody-drug conjugate is tisotumab vedotin. In some embodiments, the subject has not been previously treated for the bladder cancer. In some embodiments, the subject received at least one previous treatment for the bladder cancer. In some embodiments, the subject received prior systemic therapy for the bladder cancer. In some embodiments, the subject experienced disease progression on or after the systemic therapy. In some embodiments, the subject received no more than 3 rounds of prior systemic therapy. In some embodiments, the subject received 1, 2 or 3 rounds of prior systemic therapy. In some embodiments, the subject received 1 round of prior systemic therapy. In some embodiments, the subject received 2 rounds of prior systemic therapy. In some embodiments, the subject received 3 rounds of prior systemic therapy. In some embodiments, the subject has been previously treated with a platinum-based therapy. In some embodiments, the platinum-based therapy is selected from the group consisting of carboplatin, cisplatin, oxaliplatin, nedaplatin, triplatin tetranitrate, phenanthriplatin, picoplatin and satraplatin. In some embodiments, the platinum-based therapy is carboplatin. In some embodiments, the platinum-based therapy is cisplatin. In some embodiments, the platinum-based therapy is oxaliplatin. In some embodiments, the platinum-based therapy is nedaplatin. In some embodiments, the platinum-based therapy is triplatin tetranitrate. In some embodiments, the platinum-based therapy is phenanthriplatin. In some embodiments, the platinum-based therapy is picoplatin. In some embodiments, the platinum-based therapy is satraplatin. In some embodiments, the subject has previously undergone surgery or radiation therapy for the bladder cancer. In some embodiments, the subject has previously undergone surgery for the bladder cancer. In some embodiments, the subject has previously undergone radiation therapy for the bladder cancer. In some embodiments, the bladder cancer is an advanced stage cancer. In some embodiments, the advanced stage cancer is a stage 3 or 4 cancer. In some embodiments, the advanced stage cancer is a metastatic cancer. In some embodiments, the bladder cancer is a recurrent cancer. In some embodiments, the subject received prior treatment with standard of care therapy for the cancer and failed the prior treatment. Ina particular embodiment, the subject is a human.

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the bladder cancer cells from the subject express TF. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the bladder cancer cells from the subject express TF. In some embodiments, the percentage of cells that express TF is determined using immunohistochemistry (IHC). In some embodiments, the percentage of cells that express TF is determined using flow cytometry. In some embodiments, the percentage of cells that express TF is determined using an enzyme-linked immunosorbent assay (ELISA).

F. Endometrial Cancer

Endometrial cancer is the most common gynecologic malignancy in the United States, accounting for 6% of cancers in women. In 2017, an estimated 61,380 women were diagnosed with endometrial cancer, and approximately 11,000 died from this disease. From 1987 to 2008, there was a 50% increase in the incidence of endometrial cancer, with an approximate 300% increase in the number of associated deaths. Endometrial adenocarcinomas can be classified into two histologic categories-type 1 or type 2. Approximately 70-80% of new cases are classified as type 1 endometrial carcinomas, which are of endometrioid histology, lower grade, and often confined to the uterus at diagnosis. These tumors are estrogen-mediated, and often, women diagnosed with type 1 endometrial carcinomas are obese, with excess endogenous estrogen production. Type 1 carcinomas (estrogen dependent) have high rates ofK-ras and PTEN loss or mutation, as well as defects in mismatch repair genes, which lead to microsatellite instability (MSI). Type 2 (non-estrogen dependent) carcinomas are higher-grade adenocarcinomas and are of non-endometrioid histology, occurring in older, leaner women, although an association with increasing body mass index (BMI) has been observed. Type 2 cancers have p53 mutations, may have overexpression of human epidermal growth factor receptor 2 (HER-2/neu), and show aneuploidy. Although there are many chemotherapeutic and targeted therapy agents approved for ovarian, fallopian tube and primary peritoneal cancers, since the 1971 approval of megestrol acetate for the palliative treatment of advanced endometrial cancer, only pembrolizumab has been Food and Drug Administration (FDA)-approved for high microsatellite instability (MSI-H) or mismatch repair deficient (dMMR) endometrial cancer; this highlights the need for new therapies to treat advanced, recurrent, metastatic endometrial cancer.

The invention provides methods for treating endometrial cancer with an antibody-drug conjugate described herein. In one aspect, the antibody-drug conjugates described herein are for use in a method of treating endometrial cancer in a subject. In one aspect, the antibody-drug conjugate is tisotumab vedotin. In some embodiments, the subject has not been previously treated for the endometrial cancer. In some embodiments, the subject received at least one previous treatment for the endometrial cancer. In some embodiments, the subject received prior systemic therapy for the endometrial cancer. In some embodiments, the subject experienced disease progression on or after the systemic therapy. In some embodiments, the subject received no more than 3 rounds of prior systemic therapy. In some embodiments, the subject received 1, 2 or 3 rounds of prior systemic therapy. In some embodiments, the subject received 1 round of prior systemic therapy. In some embodiments, the subject received 2 rounds of prior systemic therapy. In some embodiments, the subject received 3 rounds of prior systemic therapy. In some embodiments, the subject has been previously treated with one or more agents selected from the group consisting of a platinum-based therapy, hormone therapy, and a checkpoint inhibitor. In some embodiments, the subject has been previously treated with a platinum-based therapy. In some embodiments, the platinum-based therapy is selected from the group consisting of carboplatin, cisplatin, oxaliplatin, nedaplatin, triplatin tetranitrate, phenanthriplatin, picoplatin and satraplatin. In some embodiments, the platinum-based therapy is carboplatin. In some embodiments, the platinum-based therapy is cisplatin. In some embodiments, the platinum-based therapy is oxaliplatin. In some embodiments, the platinum-based therapy is nedaplatin. In some embodiments, the platinum-based therapy is triplatin tetranitrate. In some embodiments, the platinum-based therapy is phenanthriplatin. In some embodiments, the platinum-based therapy is picoplatin. In some embodiments, the platinum-based therapy is satraplatin. In some embodiments, the subject has been previously treated with a hormone therapy. In some embodiments, the hormone therapy is selected from the group consisting of a progestin, tamoxifen, a luteinizing hormone-releasing hormone agonist, and an aromatase inhibitor. In some embodiments, the hormone therapy is a progestin. In some embodiments, the progestin is medroxyprogesterone acetate. In some embodiments, the progestin is megestrol acetate. In some embodiments, the hormone therapy is tamoxifen. In some embodiments, the hormone therapy is a luteinizing hormone-releasing hormone agonist. In some embodiments, the luteinizing hormone-releasing hormone agonist is goserelin. In some embodiments, the luteinizing hormone-releasing hormone agonist is leuprolide. In some embodiments, the hormone therapy is an aromatase inhibitor. In some embodiments, the aromatase inhibitor is letrozole. In some embodiments, the aromatase inhibitor is anastrozole. In some embodiments, the aromatase inhibitor is exemestane. In some embodiments, the subject has been previously treated with a checkpoint inhibitor. In some embodiments, the checkpoint inhibitor is an inhibitor of PD-1, PD-L1, CTLA-4, PD-L2, LAG3, Tim3, 2B4, A2aR, ID02, B7-H3, B7-H4, BTLA, CD2, CD20, CD27, CD28, CD30, CD33, CD40, CD52, CD70, CD80, CD86, CD112, CD137, CD 160, CD226, CD276, DR3, OX-40, GAL9, GITR, ICOS, HVEM, IDOI, KIR, LAIR, LIGHT, MARCO, PS, SLAM, TIGIT, VISTA, and/or VTCN1. In some embodiments, the checkpoint inhibitor is an inhibitor of PD-1, PD-L1 and/or CTLA-4. In some embodiments, the checkpoint inhibitor is an inhibitor of PD-1. In some embodiments, the checkpoint inhibitor is selected from the group consisting of nivolumab (OPDIVO®, BMS-936558, MDX-1106 or MK-34775), pembrolizumab (KEYTRUDA®, MK-3475), pidilizumab (CT-011) and cemiplimab (REGN2810). In some embodiments, the checkpoint inhibitor is an inhibitor of PD-L1. In some embodiments, the checkpoint inhibitor is selected from the group consisting of atezolizumab (TECENTRIQ®, MPDL3280A), avelumab (BAVENCIO®), durvalumab and BMS-936559. In some embodiments, the checkpoint inhibitor is an inhibitor of CTLA-4. In some embodiments, the checkpoint inhibitor is selected from the group consisting of ipilimumab and tremelimumab. In some embodiments, the subject has been previously treated with doxorubicin. In some embodiments, the subject has been previously treated with paclitaxel. In some embodiments, the subject has previously undergone surgery or radiation therapy for the endometrial cancer. In some embodiments, the subject has previously undergone surgery for the endometrial cancer. In some embodiments, the subject has previously undergone radiation therapy for the endometrial cancer. In some embodiments, the endometrial cancer is an advanced stage cancer. In some embodiments, the advanced stage cancer is a stage 3 or 4 cancer. In some embodiments, the advanced stage cancer is a metastatic cancer. In some embodiments, the endometrial cancer is a recurrent cancer. In some embodiments, the subject received prior treatment with standard of care therapy for the cancer and failed the prior treatment. Ina particular embodiment, the subject is a human.

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the endometrial cancer cells from the subject express TF. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the endometrial cancer cells from the subject express TF. In some embodiments, the percentage of cells that express TF is determined using immunohistochemistry (IHC). In some embodiments, the percentage of cells that express TF is determined using flow cytometry. In some embodiments, the percentage of cells that express TF is determined using an enzyme-linked immunosorbent assay (ELISA).

G. Esophageal Cancer

Esophageal cancer is the sixth leading cause of cancer-related mortality worldwide due to its overall poor prognosis. The global age-standardized incidence rate of esophageal squamous cell carcinoma (ESCC) is 1.4-13.6 per 100,000 people. Esophageal cancer is estimated to be responsible for 15,690 deaths and 16,940 new cases in the United States in 2016. The majority of patients present with locally advanced or systemic disease and outcomes remain poor despite advances in treatment. More effective treatments for these patients with locally advanced or systemic disease are urgently needed.

The invention provides methods for treating esophageal cancer with an antibody-drug conjugate described herein. In one aspect, the antibody-drug conjugates described herein are for use in a method of treating esophageal cancer in a subject. In one aspect, the antibody-drug conjugate is tisotumab vedotin. In some embodiments, the subject has not been previously treated for the esophageal cancer. In some embodiments, the subject received at least one previous treatment for the esophageal cancer. In some embodiments, the subject received prior systemic therapy for the esophageal cancer. In some embodiments, the subject experienced disease progression on or after the systemic therapy. In some embodiments, the subject received no more than 3 rounds of prior systemic therapy. In some embodiments, the subject received 1, 2 or 3 rounds of prior systemic therapy. In some embodiments, the subject received 1 round of prior systemic therapy. In some embodiments, the subject received 2 rounds of prior systemic therapy. In some embodiments, the subject received 3 rounds of prior systemic therapy. In some embodiments, the subject has been previously treated with one or more agents selected from the group consisting of a platinum-based therapy and a checkpoint inhibitor. In some embodiments, the subject has been previously treated with a platinum-based therapy. In some embodiments, the platinum-based therapy is selected from the group consisting of carboplatin, cisplatin, oxaliplatin, nedaplatin, triplatin tetranitrate, phenanthriplatin, picoplatin and satraplatin. In some embodiments, the platinum-based therapy is carboplatin. In some embodiments, the platinum-based therapy is cisplatin. In some embodiments, the platinum-based therapy is oxaliplatin. In some embodiments, the platinum-based therapy is nedaplatin. In some embodiments, the platinum-based therapy is triplatin tetranitrate. In some embodiments, the platinum-based therapy is phenanthriplatin. In some embodiments, the platinum-based therapy is picoplatin. In some embodiments, the platinum-based therapy is satraplatin. In some embodiments, the subject has been previously treated with a checkpoint inhibitor. In some embodiments, the checkpoint inhibitor is an inhibitor of PD-1, PD-L1, CTLA-4, PD-L2, LAG3, Tim3, 2B4, A2aR, ID02, B7-H3, B7-H4, BTLA, CD2, CD20, CD27, CD28, CD30, CD33, CD40, CD52, CD70, CD80, CD86, CD112, CD137, CD 160, CD226, CD276, DR3, OX-40, GAL9, GITR, ICOS, HVEM, IDOI, KIR, LAIR, LIGHT, MARCO, PS, SLAM, TIGIT, VISTA, and/or VTCN1. In some embodiments, the checkpoint inhibitor is an inhibitor of PD-1, PD-L1 and/or CTLA-4. In some embodiments, the checkpoint inhibitor is an inhibitor of PD-1. In some embodiments, the checkpoint inhibitor is selected from the group consisting of nivolumab (OPDIVO®, BMS-936558, MDX-1106 or MK-34775), pembrolizumab (KEYTRUDA®, MK-3475), pidilizumab (CT-011) and cemiplimab (REGN2810). In some embodiments, the checkpoint inhibitor is an inhibitor of PD-L1. In some embodiments, the checkpoint inhibitor is selected from the group consisting of atezolizumab (TECENTRIQ®, MPDL3280A), avelumab (BAVENCIO®), durvalumab and BMS-936559. In some embodiments, the checkpoint inhibitor is an inhibitor of CTLA-4. In some embodiments, the checkpoint inhibitor is selected from the group consisting of ipilimumab and tremelimumab. In some embodiments, the subject has been previously treated with one or more agents selected from the group consisting of ramucirumab, paclitaxel, 5-fluorouracil, docetaxel, irinotecan, capecitabine and trastuzumab. In some embodiments, the subject has been previously treated with ramucirumab. In some embodiments, the subject has been previously treated with paclitaxel. In some embodiments, the subject has been previously treated with 5-fluorouracil. In some embodiments, the subject has been previously treated with docetaxel. In some embodiments, the subject has been previously treated with irinotecan. In some embodiments, the subject has been previously treated with capecitabine. In some embodiments, the subject has been previously treated with trastuzumab. In some embodiments, the subject has previously undergone surgery, radiation therapy, or endoscopic mucosal resection for the esophageal cancer. In some embodiments, the subject has previously undergone surgery for the esophageal cancer. In some embodiments, the subject has previously undergone radiation therapy for the esophageal cancer. In some embodiments, the subject has previously undergone endoscopic mucosal resection for the esophageal cancer. In some embodiments, the esophageal cancer is an advanced stage cancer. In some embodiments, the advanced stage cancer is a stage 3 or 4 cancer. In some embodiments, the advanced stage cancer is a metastatic cancer. In some embodiments, the esophageal cancer is a recurrent cancer. In some embodiments, the subject received prior treatment with standard of care therapy for the cancer and failed the prior treatment. In a particular embodiment, the subject is a human.

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the esophageal cancer cells from the subject express TF. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the esophageal cancer cells from the subject express TF. In some embodiments, the percentage of cells that express TF is determined using immunohistochemistry (IHC). In some embodiments, the percentage of cells that express TF is determined using flow cytometry. In some embodiments, the percentage of cells that express TF is determined using an enzyme-linked immunosorbent assay (ELISA).

H. Prostate Cancer

Prostate cancer is the most common non-cutaneous malignancy in males, with a projected 161,360 incident cases and 26,730 deaths estimated in the United States in 2017 alone. Curative modalities for localized prostate cancer include surgery and/or radiation therapy, with or without androgen deprivation therapy. While contemporary treatment methods, such as intensity-modulated radiotherapy, are used to deliver radiation with high accuracy, defining the position and the extent of the tumor is still quite challenging. Other issues in the treatment of the radiotherapy patient include the choice of the radiotherapy technique (hypo- or standard fractionation) and the use and length of androgen deprivation therapy. More effective treatments are needed, especially for patients with advanced and metastatic prostate cancer.

The invention provides methods for treating prostate cancer with an antibody-drug conjugate described herein. In one aspect, the antibody-drug conjugates described herein are for use in a method of treating prostate cancer in a subject. In one aspect, the antibody-drug conjugate is tisotumab vedotin. In some embodiments, the subject has not been previously treated for the prostate cancer. In some embodiments, the subject received at least one previous treatment for the prostate cancer. In some embodiments, the subject received prior systemic therapy for the prostate cancer. In some embodiments, the subject experienced disease progression on or after the systemic therapy. In some embodiments, the subject received no more than 3 rounds of prior systemic therapy. In some embodiments, the subject received 1, 2 or 3 rounds of prior systemic therapy. In some embodiments, the subject received 1 round of prior systemic therapy. In some embodiments, the subject received 2 rounds of prior systemic therapy. In some embodiments, the subject received 3 rounds of prior systemic therapy. In some embodiments, the prostate cancer is castration-resistant prostate cancer. In some embodiments, the subject has experienced bone metastases. In some embodiments, the prostate cancer has metastasized to a bone. In some embodiments, the subject has been previously treated with one or more agents selected from the group consisting of androgen deprivation therapy, a luteinizing hormone-releasing hormone agonist, a luteinizing hormone-releasing hormone antagonist, a CYP17 inhibitor, and an anti-androgen. In some embodiments, the subject has been previously treated with androgen deprivation therapy. In some embodiments, the subject has been previously treated with a luteinizing hormone-releasing hormone agonist. In some embodiments, the luteinizing hormone-releasing hormone agonist is selected from the group consisting of leuprolide, goserelin, triptorelin and histrelin. In some embodiments, the luteinizing hormone-releasing hormone agonist is leuprolide. In some embodiments, the luteinizing hormone-releasing hormone agonist is goserelin. In some embodiments, the luteinizing hormone-releasing hormone agonist is triptorelin. In some embodiments, the luteinizing hormone-releasing hormone agonist is histrelin. In some embodiments, the subject has been previously treated with a luteinizing hormone-releasing hormone antagonist. In some embodiments, the luteinizing hormone-releasing hormone antagonist is degarelix. In some embodiments, the subject has been previously treated with a CYP17 inhibitor. In some embodiments, the CYP17 inhibitor is abiraterone. In some embodiments, the subject has been previously treated with an anti-androgen. In some embodiments, the anti-androgen is selected from the group consisting of flutamide, bicalutamide, nilutamide, enzalutamide and apalutamide. In some embodiments, the anti-androgen is flutamide. In some embodiments, the anti-androgen is bicalutamide. In some embodiments, the anti-androgen is nilutamide. In some embodiments, the anti-androgen is enzalutamide. In some embodiments, the anti-androgen is apalutamide. In some embodiments, the subject has been previously treated with one or more agents selected from the group consisting of docetaxel, prednisone and cabazitaxel. In some embodiments, the subject has been previously treated with docetaxel. In some embodiments, the subject has been previously treated with prednisone. In some embodiments, the subject has been previously treated with cabazitaxel. In some embodiments, the subject has previously undergone surgery or radiation therapy for the prostate cancer. In some embodiments, the subject has previously undergone surgery for the prostate cancer. In some embodiments, the subject has previously undergone radiation therapy for the prostate cancer. In some embodiments, the prostate cancer is an advanced stage cancer. In some embodiments, the advanced stage cancer is a stage 3 or 4 cancer. In some embodiments, the advanced stage cancer is a metastatic cancer. In some embodiments, the prostate cancer is a recurrent cancer. In some embodiments, the subject received prior treatment with standard of care therapy for the cancer and failed the prior treatment. In a particular embodiment, the subject is a human.

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the prostate cancer cells from the subject express TF. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the prostate cancer cells from the subject express TF. In some embodiments, the percentage of cells that express TF is determined using immunohistochemistry (IHC). In some embodiments, the percentage of cells that express TF is determined using flow cytometry. In some embodiments, the percentage of cells that express TF is determined using an enzyme-linked immunosorbent assay (ELISA).

I. Routes of Administration

An anti-TF antibody-drug conjugate or antigen-binding fragment thereof described herein can be administered by any suitable route and mode. Suitable routes of administering antibody-drug conjugate of the present invention are well known in the art and may be selected by those of ordinary skill in the art. In one embodiment, the antibody-drug conjugate is administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of an anti-TF antibody-drug conjugate or antigen-binding fragment described herein is intravenous injection or infusion. In some embodiments, the route of administration of an anti-TF antibody-drug conjugate or antigen-binding fragment described herein is intravenous infusion.

J. Dosage and Frequency of Administration

In one aspect, the present invention provides for methods of treating a subject with colorectal cancer, non-small cell lung cancer, pancreatic cancer, head and neck cancer, bladder cancer, endometrial cancer, esophageal cancer or prostate cancer as described herein with a particular dose of an anti-TF antibody-drug conjugate or antigen-binding fragment thereof as described herein, wherein the subject is administered the antibody-drug conjugate or antigen-binding fragment thereof as described herein with a particular frequency.

In one embodiment of the methods or uses or product for uses provided herein, an anti-TF antibody-drug conjugate or antigen-binding fragment thereof as described herein is administered to the subject at a dose ranging from about 0.9 mg/kg to about 2.1 mg/kg of the subject's body weight. In certain embodiments, the dose is about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg or about 2.1 mg/kg. In one embodiment, the dose is about 2.0 mg/kg. In certain embodiments, the dose is 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg or 2.1 mg/kg. In one embodiment, the dose is 2.0 mg/kg. In some embodiments, the dose is 2.0 mg/kg and the anti-TF antibody-drug conjugate is tisotumab vedotin. In some embodiments, for a subject weighing more than 100 kg, the dose of the anti-TF antibody-drug conjugate administered is the amount that would be administered if the subject weighed 100 kg. In some embodiments, for a subject weighing more than 100 kg, the dose of the anti-TF antibody-drug conjugate administered is 200 mg.

In one embodiment of the methods or uses or product for uses provided herein, an anti-TF antibody-drug conjugate or antigen-binding fragment thereof as described herein is administered to the subject once about every 1 to 4 weeks. In certain embodiments, an anti-TF antibody-drug conjugate or antigen-binding fragment thereof as described herein is administered once about every 1 week, once about every 2 weeks, once about every 3 weeks or once about every 4 weeks. In one embodiment, an anti-TF antibody-drug conjugate or antigen-binding fragment thereof as described herein is administered once about every 3 weeks. In one embodiment, an anti-TF antibody-drug conjugate or antigen-binding fragment thereof as described herein is administered once every 3 weeks. In some embodiments, the dose is about 0.9 mg/kg and is administered once about every 1 week. In some embodiments, the dose is about 0.9 mg/kg and is administered once about every 2 weeks. In some embodiments, the dose is about 0.9 mg/kg and is administered once about every 3 weeks. In some embodiments, the dose is about 0.9 mg/kg and is administered once about every 4 weeks. In some embodiments, the dose is about 1.0 mg/kg and is administered once about every 1 week. In some embodiments, the dose is about 1.0 mg/kg and is administered once about every 2 weeks. In some embodiments, the dose is about 1.0 mg/kg and is administered once about every 3 weeks. In some embodiments, the dose is about 1.0 mg/kg and is administered once about every 4 weeks. In some embodiments, the dose is about 1.1 mg/kg and is administered once about every 1 week. In some embodiments, the dose is about 1.1 mg/kg and is administered once about every 2 weeks. In some embodiments, the dose is about 1.1 mg/kg and is administered once about every 3 weeks. In some embodiments, the dose is about 1.1 mg/kg and is administered once about every 4 weeks. In some embodiments, the dose is about 1.2 mg/kg and is administered once about every 1 week. In some embodiments, the dose is about 1.2 mg/kg and is administered once about every 2 weeks. In some embodiments, the dose is about 1.2 mg/kg and is administered once about every 3 weeks. In some embodiments, the dose is about 1.2 mg/kg and is administered once about every 4 weeks. In some embodiments, the dose is about 1.3 mg/kg and is administered once about every 1 week. In some embodiments, the dose is about 1.3 mg/kg and is administered once about every 2 weeks. In some embodiments, the dose is about 1.3 mg/kg and is administered once about every 3 weeks. In some embodiments, the dose is about 1.3 mg/kg and is administered once about every 4 weeks. In some embodiments, the dose is about 1.4 mg/kg and is administered once about every 1 week. In some embodiments, the dose is about 1.4 mg/kg and is administered once about every 2 weeks. In some embodiments, the dose is about 1.4 mg/kg and is administered once about every 3 weeks. In some embodiments, the dose is about 1.4 mg/kg and is administered once about every 4 weeks. In some embodiments, the dose is about 1.5 mg/kg and is administered once about every 1 week. In some embodiments, the dose is about 1.5 mg/kg and is administered once about every 2 weeks. In some embodiments, the dose is about 1.5 mg/kg and is administered once about every 3 weeks. In some embodiments, the dose is about 1.5 mg/kg and is administered once about every 4 weeks. In some embodiments, the dose is about 1.6 mg/kg and is administered once about every 1 week. In some embodiments, the dose is about 1.6 mg/kg and is administered once about every 2 weeks. In some embodiments, the dose is about 1.6 mg/kg and is administered once about every 3 weeks. In some embodiments, the dose is about 1.6 mg/kg and is administered once about every 4 weeks. In some embodiments, the dose is about 1.7 mg/kg and is administered once about every 1 week. In some embodiments, the dose is about 1.7 mg/kg and is administered once about every 2 weeks. In some embodiments, the dose is about 1.7 mg/kg and is administered once about every 3 weeks. In some embodiments, the dose is about 1.7 mg/kg and is administered once about every 4 weeks. In some embodiments, the dose is about 1.8 mg/kg and is administered once about every 1 week. In some embodiments, the dose is about 1.8 mg/kg and is administered once about every 2 weeks. In some embodiments, the dose is about 1.8 mg/kg and is administered once about every 3 weeks. In some embodiments, the dose is about 1.8 mg/kg and is administered once about every 4 weeks. In some embodiments, the dose is about 1.9 mg/kg and is administered once about every 1 week. In some embodiments, the dose is about 1.9 mg/kg and is administered once about every 2 weeks. In some embodiments, the dose is about 1.9 mg/kg and is administered once about every 3 weeks. In some embodiments, the dose is about 1.9 mg/kg and is administered once about every 4 weeks. In some embodiments, the dose is about 2.0 mg/kg and is administered once about every 1 week. In some embodiments, the dose is about 2.0 mg/kg and is administered once about every 2 weeks. In some embodiments, the dose is about 2.0 mg/kg and is administered once about every 3 weeks. In some embodiments, the dose is about 2.0 mg/kg and is administered once about every 4 weeks. In some embodiments, the dose is about 2.1 mg/kg and is administered once about every 1 week. In some embodiments, the dose is about 2.1 mg/kg and is administered once about every 2 weeks. In some embodiments, the dose is about 2.1 mg/kg and is administered once about every 3 weeks. In some embodiments, the dose is about 2.1 mg/kg and is administered once about every 4 weeks. In some embodiments, the dose is 0.9 mg/kg and is administered once about every 1 week. In some embodiments, the dose is 0.9 mg/kg and is administered once about every 2 weeks. In some embodiments, the dose is 0.9 mg/kg and is administered once about every 3 weeks. In some embodiments, the dose is 0.9 mg/kg and is administered once about every 4 weeks. In some embodiments, the dose is 1.0 mg/kg and is administered once about every 1 week. In some embodiments, the dose is 1.0 mg/kg and is administered once about every 2 weeks. In some embodiments, the dose is 1.0 mg/kg and is administered once about every 3 weeks. In some embodiments, the dose is 1.0 mg/kg and is administered once about every 4 weeks. In some embodiments, the dose is 1.1 mg/kg and is administered once about every 1 week. In some embodiments, the dose is 1.1 mg/kg and is administered once about every 2 weeks. In some embodiments, the dose is 1.1 mg/kg and is administered once about every 3 weeks. In some embodiments, the dose is 1.1 mg/kg and is administered once about every 4 weeks. In some embodiments, the dose is 1.2 mg/kg and is administered once about every 1 week. In some embodiments, the dose is 1.2 mg/kg and is administered once about every 2 weeks. In some embodiments, the dose is 1.2 mg/kg and is administered once about every 3 weeks. In some embodiments, the dose is 1.2 mg/kg and is administered once about every 4 weeks. In some embodiments, the dose is 1.3 mg/kg and is administered once about every 1 week. In some embodiments, the dose is 1.3 mg/kg and is administered once about every 2 weeks. In some embodiments, the dose is 1.3 mg/kg and is administered once about every 3 weeks. In some embodiments, the dose is 1.3 mg/kg and is administered once about every 4 weeks. In some embodiments, the dose is 1.4 mg/kg and is administered once about every 1 week. In some embodiments, the dose is 1.4 mg/kg and is administered once about every 2 weeks. In some embodiments, the dose is 1.4 mg/kg and is administered once about every 3 weeks. In some embodiments, the dose is 1.4 mg/kg and is administered once about every 4 weeks. In some embodiments, the dose is 1.5 mg/kg and is administered once about every 1 week. In some embodiments, the dose is 1.5 mg/kg and is administered once about every 2 weeks. In some embodiments, the dose is 1.5 mg/kg and is administered once about every 3 weeks. In some embodiments, the dose is 1.5 mg/kg and is administered once about every 4 weeks. In some embodiments, the dose is 1.6 mg/kg and is administered once about every 1 week. In some embodiments, the dose is 1.6 mg/kg and is administered once about every 2 weeks. In some embodiments, the dose is 1.6 mg/kg and is administered once about every 3 weeks. In some embodiments, the dose is 1.6 mg/kg and is administered once about every 4 weeks. In some embodiments, the dose is 1.7 mg/kg and is administered once about every 1 week. In some embodiments, the dose is 1.7 mg/kg and is administered once about every 2 weeks. In some embodiments, the dose is 1.7 mg/kg and is administered once about every 3 weeks. In some embodiments, the dose is 1.7 mg/kg and is administered once about every 4 weeks. In some embodiments, the dose is 1.8 mg/kg and is administered once about every 1 week. In some embodiments, the dose is 1.8 mg/kg and is administered once about every 2 weeks. In some embodiments, the dose is 1.8 mg/kg and is administered once about every 3 weeks. In some embodiments, the dose is 1.8 mg/kg and is administered once about every 4 weeks. In some embodiments, the dose is 1.9 mg/kg and is administered once about every 1 week. In some embodiments, the dose is 1.9 mg/kg and is administered once about every 2 weeks. In some embodiments, the dose is 1.9 mg/kg and is administered once about every 3 weeks. In some embodiments, the dose is 1.9 mg/kg and is administered once about every 4 weeks. In some embodiments, the dose is 2.0 mg/kg and is administered once about every 1 week. In some embodiments, the dose is 2.0 mg/kg and is administered once about every 2 weeks. In some embodiments, the dose is 2.0 mg/kg and is administered once about every 3 weeks. In some embodiments, the dose is 2.0 mg/kg and is administered once about every 4 weeks. In some embodiments, the dose is 2.1 mg/kg and is administered once about every 1 week. In some embodiments, the dose is 2.1 mg/kg and is administered once about every 2 weeks. In some embodiments, the dose is 2.1 mg/kg and is administered once about every 3 weeks. In some embodiments, the dose is 2.1 mg/kg and is administered once about every 4 weeks. In some embodiments, the dose is 2.0 mg/kg and is administered once about every 3 weeks (e.g., 3 days). In some embodiments, the dose is 2.0 mg/kg and is administered once every 3 weeks. In some embodiments, the dose is 2.0 mg/kg and is administered once every 3 weeks and the antibody-drug conjugate is tisotumab vedotin. In some embodiments, the dose is 2.0 mg/kg and is administered once every 3 weeks and the antibody-drug conjugate is tisotumab vedotin and the dose is decreased to 1.3 mg/kg if one or more adverse events occur. In some embodiments, the dose is 1.3 mg/kg and is administered once every 3 weeks and the antibody-drug conjugate is tisotumab vedotin and the dose is decreased to 0.9 mg/kg if one or more adverse events occur. In some embodiments, for a subject weighing more than 100 kg, the dose of the anti-TF antibody-drug conjugate administered is the amount that would be administered if the subject weighed 100 kg. In some embodiments, for a subject weighing more than 100 kg, the dose of the anti-TF antibody-drug conjugate administered is 200 mg.

In one embodiment of the methods or uses or product for uses provided herein, an anti-TF antibody-drug conjugate or antigen-binding fragment thereof as described herein is administered to the subject at a flat dose ranging from about 50 mg to about 200 mg such as at a flat dose of about 50 mg or a flat dose of about 60 mg or a flat dose of about 70 mg or a flat dose of about 80 mg or a flat dose of about 90 mg or a flat dose of about 100 mg or a flat dose of about 110 mg or a flat dose of about 120 mg or a flat dose of about 130 mg or a flat dose of about 140 mg or a flat dose of about 150 mg or a flat dose of about 160 mg or a flat dose of about 170 mg or a flat dose of about 180 mg or a flat dose of about 190 mg or a flat dose of about 200 mg. In some embodiments, the flat dose is administered to the subject once about every 1 to 4 weeks. In certain embodiments, the flat dose is administered to the subject once about every 1 week, once about every 2 weeks, once about every 3 weeks or once about every 4 weeks. In some embodiments, the flat dose is administered to the subject once about every 3 weeks (e.g., 3 days). In some embodiments, the flat dose is administered to the subject once every 3 weeks. In some embodiments, the flat dose is administered to the subject once every 3 weeks and the antibody-drug conjugate is tisotumab vedotin.

In one embodiment of the methods or uses or product for uses provided herein, an anti-TF antibody-drug conjugate or antigen-binding fragment thereof as described herein is administered to the subject at a flat dose ranging from 50 mg to 200 mg such as at a flat dose of 50 mg or a flat dose of 60 mg or a flat dose of 70 mg or a flat dose of 80 mg or a flat dose of 90 mg or a flat dose of 100 mg or a flat dose of 110 mg or a flat dose of 120 mg or a flat dose of 130 mg or a flat dose of 140 mg or a flat dose of 150 mg or a flat dose of 160 mg or a flat dose of 170 mg or a flat dose of 180 mg or a flat dose of 190 mg or a flat dose of 200 mg. In some embodiments, the flat dose is administered to the subject once about every 1 to 4 weeks. In certain embodiments, the flat dose is administered to the subject once about every 1 week, once about every 2 weeks, once about every 3 weeks or once about every 4 weeks. In some embodiments, the flat dose is administered to the subject once about every 3 weeks (e.g., 3 days). In some embodiments, the flat dose is administered to the subject once every 3 weeks. In some embodiments, the flat dose is administered to the subject once every 3 weeks and the antibody-drug conjugate is tisotumab vedotin.

In some embodiments, a method of treatment or use or product for use described herein further comprises the administration of one or more additional therapeutic agents. In some embodiments, the one or more additional therapeutic agents are administered simultaneously with an anti-TF antibody-drug conjugate or antigen-binding fragment thereof as described herein, such as tisotumab vedotin. In some embodiments, the one or more additional therapeutic agents and an anti-TF antibody-drug conjugate or antigen-binding fragment thereof as described herein are administered sequentially. In some embodiments, simultaneous means that the anti-TF antibody-drug conjugate and the one or more additional therapeutic agents are administered to the subject less than one hour apart, such as less than about 30 minutes apart, less than about 15 minutes apart, less than about 10 minutes apart or less than about 5 minutes apart. In some embodiments, sequential administration means that the anti-TF antibody-drug conjugate and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart.

K. Treatment Outcome

In one aspect, a method of treating colorectal cancer, non-small cell lung cancer, pancreatic cancer, head and neck cancer, bladder cancer, endometrial cancer, esophageal cancer or prostate cancer with an anti-TF antibody-drug conjugate or antigen-binding fragment thereof as described herein, such as e.g., tisotumab vedotin, results in an improvement in one or more therapeutic effects in the subject after administration of the antibody-drug conjugate relative to a baseline. In some embodiments, the one or more therapeutic effects is the size of the tumor derived from the cancer (e.g., colorectal cancer, non-small cell lung cancer, pancreatic cancer, head and neck cancer, bladder cancer, endometrial cancer, esophageal cancer or prostate cancer), the objective response rate, the duration of response, the time to response, progression free survival, overall survival, or any combination thereof. In one embodiment, the one or more therapeutic effects is the size of the tumor derived from the cancer. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression. In one embodiment, the one or more therapeutic effects is a reduction in prostate specific antigen level.

In one embodiment of the methods or uses or product for uses provided herein, response to treatment with an anti-TF antibody-drug conjugate or antigen-binding fragment thereof as described herein, such as e.g., tisotumab vedotin, may include the following criteria (RECIST Criteria 1.1):

| | Category | Criteria |
|---|---|---|
| Based on | Complete | Disappearance of all target lesions. Any pathological |
| target lesions | Response (CR) | lymph nodes must have reduction in short axis to |
| | | <10 mm. |
| | Partial Response | ≥30% decrease in the sum of the longest diameter |
| | (PR) | (LD) of target lesions, taking as reference the |
| | | baseline sum of LDs. |
| | Stable Disease | Neither sufficient shrinkage to qualify for PR nor |
| | (SD) | sufficient increase to qualify for PD, taking as |
| | | reference the smallest sum of LDs while in trial. |
| | Progressive | ≥20% (and ≥5 mm) increase in the sum of the LDs |
| | Disease (PD) | of target lesions, taking as reference the smallest sum |
| | | of the target LDs recorded while in trial or the |
| | | appearance of one or more new lesions. |
| Based on non- | CR | Disappearance of all non-target lesions and |
| target lesions | | normalization of tumor marker level. All lymph |
| | | nodes must be non-pathological in size |
| | | (<10 mm short axis). |
| | SD | Persistence of one or more non-target lesion(s) |
| | | or/and maintenance of tumor marker level |
| | | above the normal limits. |
| | PD | Appearance of one or more new lesions and/or |
| | | unequivocal progression of existing non-target |
| | | lesions. |

In one embodiment of the methods or uses or product for uses provided herein, the effectiveness of treatment with an anti-TF antibody-drug conjugate or antigen-binding fragment thereof described herein, such as e.g., tisotumab vedotin, is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment with an anti-TF antibody-drug conjugate or antigen-binding fragment thereof described herein, such as e.g., tisotumab vedotin, is assessed by measuring the size of a tumor derived from the cancer (e.g., colorectal cancer, non-small cell lung cancer, pancreatic cancer, head and neck cancer, bladder cancer, endometrial cancer, esophageal cancer or prostate cancer). In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the anti-TF antibody-drug conjugate. In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the anti-TF antibody-drug conjugate. In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In one embodiment, the size of a tumor derived from the cancer is measured by magnetic resonance imaging (MRI). In one embodiment, the size of a tumor derived from the cancer is measured by computed tomography (CT). In one embodiment, the size of a tumor derived from the cancer is measured by positron emission tomography (PET). In one embodiment, the size of a tumor derived from the cancer is measured by ultrasound. In some embodiments, the size of a tumor derived from a colorectal cancer is measured by computed tomography (CT), positron emission tomography (PET) or magnetic resonance imaging (MRI). See Goh et al., 2014, Br. J. Radiol. 87(1034):20130811. In some embodiments, the size of a tumor derived from a non-small cell lung cancer is measured by computed tomography (CT) or positron emission tomography (PET). See Aydin et al., 2013, Diagn. Interv. Radiol. 19(4):271-8. In some embodiments, the size of a tumor derived from a pancreatic cancer is measured by computed tomography (CT), magnetic resonance imaging (MRI), ultrasound or positron emission tomography (PET). See Wolfgang et al., 2013, CA Cancer J. Clin. 63(5)318-348. In some embodiments, the size of a tumor derived from a head and neck cancer is measured by computed tomography (CT), magnetic resonance imaging (MRI), ultrasound or positron emission tomography (PET). See Nooij et al., 2018, Curr. Radiol. Rep. 6(1):2. In some embodiments, the size of a tumor derived from a bladder cancer is measured by positron emission tomography (PET). See Vlachostergios et al., 2018, Bladder Cancer 4(3):247-259. In some embodiments, the size of a tumor derived from an endometrial cancer is measured by ultrasound, magnetic resonance imaging (MRI) or computed tomography (CT). See Nyen et al., 2018, Int. J. Mol. Sci. 19(8):2348. In some embodiments, the size of a tumor derived from an esophageal cancer is measured by ultrasound, computed tomography (CT) or positron emission tomography (PET). See Park and Kim, 2018, Ann. Transl. Med. 6(4):82. In some embodiments, the size of a tumor derived from a prostate cancer is measured by ultrasound, magnetic resonance imaging (MRI), computed tomography (CT) or positron emission tomography (PET). See Das et al., 2018, Indian J. Urol., 34(3):172-179.

In one embodiment of the methods or uses or product for uses provided described herein, response to treatment with an antibody-drug conjugate or antigen-binding fragment thereof described herein, such as e.g., tisotumab vedotin, promotes regression of a tumor derived from the cancer (e.g., colorectal cancer, non-small cell lung cancer, pancreatic cancer, head and neck cancer, bladder cancer, endometrial cancer, esophageal cancer or prostate cancer). In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the anti-TF antibody-drug conjugate. In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the anti-TF antibody-drug conjugate. In one embodiment, a tumor derived from the cancer regresses by at least 10% to 80%. In one embodiment, a tumor derived from the cancer regresses by at least 20% to 80%. In one embodiment, a tumor derived from the cancer regresses by at least 30% to 80%. In one embodiment, a tumor derived from the cancer regresses by at least 40% to 80%. In one embodiment, a tumor derived from the cancer regresses by at least 50% to 80%. In one embodiment, a tumor derived from the cancer regresses by at least 60% to 80%. In one embodiment, a tumor derived from the cancer regresses by at least 70% to 80%. In one embodiment, a tumor derived from the cancer regresses by at least 80%. In one embodiment, a tumor derived from the cancer regresses by at least 85%. In one embodiment, a tumor derived from the cancer regresses by at least 90%. In one embodiment, a tumor derived from the cancer regresses by at least 95%. In one embodiment, a tumor derived from the cancer regresses by at least 98%. In one embodiment, a tumor derived from the cancer regresses by at least 99%. In one embodiment, a tumor derived from the cancer regresses by 100%. In one embodiment, regression of a tumor is determined by measuring the size of the tumor by magnetic resonance imaging (MRI). In one embodiment, regression of a tumor is determined by measuring the size of the tumor by computed tomography (CT). In one embodiment, regression of a tumor is determined by measuring the size of the tumor by positron emission tomography (PET). In one embodiment, regression of a tumor is determined by measuring the size of the tumor by ultrasound. In some embodiments, regression of a tumor derived from a colorectal cancer is measured by computed tomography (CT), positron emission tomography (PET) or magnetic resonance imaging (MRI). See Goh et al., 2014, Br. J. Radiol. 87(1034):20130811. In some embodiments, regression of a tumor derived from a non-small cell lung cancer is measured by computed tomography (CT) or positron emission tomography (PET). See Aydin et al., 2013, Diagn. Interv. Radiol. 19(4):271-8. In some embodiments, regression of a tumor derived from a pancreatic cancer is measured by computed tomography (CT), magnetic resonance imaging (MRI), ultrasound or positron emission tomography (PET). See Wolfgang et al., 2013, CA Cancer J. Clin. 63(5)318-348. In some embodiments, regression of a tumor derived from a head and neck cancer is measured by computed tomography (CT), magnetic resonance imaging (MRI), ultrasound or positron emission tomography (PET). See Nooij et al., 2018, Curr. Radiol. Rep. 6(1):2. In some embodiments, regression of a tumor derived from a bladder cancer is measured by positron emission tomography (PET). See Vlachostergios et al., 2018, Bladder Cancer 4(3):247-259. In some embodiments, regression of a tumor derived from an endometrial cancer is measured by ultrasound, magnetic resonance imaging (MRI) or computed tomography (CT). See Nyen et al., 2018, Int. J. Mol. Sci. 19(8):2348. In some embodiments, regression of a tumor derived from an esophageal cancer is measured by ultrasound, computed tomography (CT) or positron emission tomography (PET). See Park and Kim, 2018, Ann. Transl. Med. 6(4):82. In some embodiments, regression of a tumor derived from a prostate cancer is measured by ultrasound, magnetic resonance imaging (MRI), computed tomography (CT) or positron emission tomography (PET). See Das et al., 2018, Indian J. Urol., 34(3):172-179.

In one embodiment of the methods or uses or product for uses described herein, response to treatment with an anti-TF antibody-drug conjugate or antigen-binding fragment thereof described herein, such as e.g., tisotumab vedotin, is assessed by measuring the time of progression free survival after administration of the anti-TF antibody-drug conjugate. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-TF antibody-drug conjugate. In some embodiments, the subject exhibits progression-free survival of at least about 6 months after administration of the anti-TF antibody-drug conjugate. In some embodiments, the subject exhibits progression-free survival of at least about one year after administration of the anti-TF antibody-drug conjugate. In some embodiments, the subject exhibits progression-free survival of at least about two years after administration of the anti-TF antibody-drug conjugate. In some embodiments, the subject exhibits progression-free survival of at least about three years after administration of the anti-TF antibody-drug conjugate. In some embodiments, the subject exhibits progression-free survival of at least about four years after administration of the anti-TF antibody-drug conjugate. In some embodiments, the subject exhibits progression-free survival of at least about five years after administration of the anti-TF antibody-drug conjugate. In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-TF antibody-drug conjugate. In some embodiments, the subject exhibits progression-free survival of at least 6 months after administration of the anti-TF antibody-drug conjugate. In some embodiments, the subject exhibits progression-free survival of at least one year after administration of the anti-TF antibody-drug conjugate. In some embodiments, the subject exhibits progression-free survival of at least two years after administration of the anti-TF antibody-drug conjugate. In some embodiments, the subject exhibits progression-free survival of at least three years after administration of the anti-TF antibody-drug conjugate. In some embodiments, the subject exhibits progression-free survival of at least four years after administration of the anti-TF antibody-drug conjugate. In some embodiments, the subject exhibits progression-free survival of at least five years after administration of the anti-TF antibody-drug conjugate.

In one embodiment of the methods or uses or product for uses described herein, response to treatment with an anti-TF antibody-drug conjugate or antigen-binding fragment thereof described herein, such as e.g., tisotumab vedotin, is assessed by measuring the time of overall survival after administration of the anti-TF antibody-drug conjugate. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-TF antibody-drug conjugate. In some embodiments, the subject exhibits overall survival of at least about 6 months after administration of the anti-TF antibody-drug conjugate. In some embodiments, the subject exhibits overall survival of at least about one year after administration of the anti-TF antibody-drug conjugate. In some embodiments, the subject exhibits overall survival of at least about two years after administration of the anti-TF antibody-drug conjugate. In some embodiments, the subject exhibits overall survival of at least about three years after administration of the anti-TF antibody-drug conjugate. In some embodiments, the subject exhibits overall survival of at least about four years after administration of the anti-TF antibody-drug conjugate. In some embodiments, the subject exhibits overall survival of at least about five years after administration of the anti-TF antibody-drug conjugate. In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-TF antibody-drug conjugate. In some embodiments, the subject exhibits overall survival of at least 6 months after administration of the anti-TF antibody-drug conjugate. In some embodiments, the subject exhibits overall survival of at least one year after administration of the anti-TF antibody-drug conjugate. In some embodiments, the subject exhibits overall survival of at least two years after administration of the anti-TF antibody-drug conjugate. In some embodiments, the subject exhibits overall survival of at least three years after administration of the anti-TF antibody-drug conjugate. In some embodiments, the subject exhibits overall survival of at least four years after administration of the anti-TF antibody-drug conjugate. In some embodiments, the subject exhibits overall survival of at least five years after administration of the anti-TF antibody-drug conjugate.

In one embodiment of the methods or uses or product for uses described herein, response to treatment with an anti-TF antibody-drug conjugate or antigen-binding fragment thereof described herein, such as e.g., tisotumab vedotin, is assessed by measuring the duration of response to the anti-TF antibody-drug conjugate after administration of the anti-TF antibody-drug conjugate. In some embodiments, the duration of response to the anti-TF antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-TF antibody-drug conjugate. In some embodiments, the duration of response to the anti-TF antibody-drug conjugate is at least about 6 months after administration of the antibody-drug conjugate. In some embodiments, the duration of response to the anti-TF antibody-drug conjugate is at least about one year after administration of the antibody-drug conjugate. In some embodiments, the duration of response to the anti-TF antibody-drug conjugate is at least about two years after administration of the antibody-drug conjugate. In some embodiments, the duration of response to the anti-TF antibody-drug conjugate is at least about three years after administration of the antibody-drug conjugate. In some embodiments, the duration of response to the anti-TF antibody-drug conjugate is at least about four years after administration of the antibody-drug conjugate. In some embodiments, the duration of response to the anti-TF antibody-drug conjugate is at least about five years after administration of the antibody-drug conjugate. In some embodiments, the duration of response to the anti-TF antibody-drug conjugate is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-TF antibody-drug conjugate. In some embodiments, the duration of response to the anti-TF antibody-drug conjugate is at least 6 months after administration of the antibody-drug conjugate. In some embodiments, the duration of response to the anti-TF antibody-drug conjugate is at least one year after administration of the antibody-drug conjugate. In some embodiments, the duration of response to the anti-TF antibody-drug conjugate is at least two years after administration of the antibody-drug conjugate. In some embodiments, the duration of response to the anti-TF antibody-drug conjugate is at least three years after administration of the antibody-drug conjugate. In some embodiments, the duration of response to the anti-TF antibody-drug conjugate is at least four years after administration of the antibody-drug conjugate. In some embodiments, the duration of response to the anti-TF antibody-drug conjugate is at least five years after administration of the antibody-drug conjugate.

In one embodiment of the methods or uses or product for uses described herein, response to treatment of prostate cancer with an anti-TF antibody-drug conjugate or antigen-binding fragment thereof described herein, such as e.g., tisotumab vedotin, is assessed by measuring prostate specific antigen (PSA) level in a blood sample from the subject. In some embodiments, the PSA levels are assessed based on the Prostate Cancer Clinical Trials Working Group Guidelines (PCWG2). See Scher et al., 2008, J. Clin. Oncol. 26(7):1148-59. In some embodiments, the subject exhibits a reduction in PSA level in a blood sample from the subject by at least about 5%, at least about 10%, at least about 15%, at least about 20%, at least about 25%, at least about 30%, at least about 35%, at least about 40%, at least about 45%, at least about 50%, at least about 60%, at least about 70%, or at least about 80% relative to the PSA level in a blood sample obtained from the subject before administration of the antibody-drug conjugate.

L. Adverse Events

In one aspect, a method of treating cancer (e.g., colorectal cancer, non-small cell lung cancer, pancreatic cancer, head and neck cancer, bladder cancer, endometrial cancer, esophageal cancer or prostate cancer) with an anti-TF antibody-drug conjugates or antigen-binding fragments thereof described herein, such as e.g., tisotumab vedotin, results in the subject developing one or more adverse events. In some embodiments, the subject is administered an additional therapeutic agent to eliminate or reduce the severity of the adverse event. In some embodiments, the one or more adverse events the subject develops is anemia, abdominal pain, hypokalemia, hyponatremia, epistaxis, fatigue, nausea, alopecia, conjunctivitis, constipation, decreased appetite, diarrhea, vomiting, peripheral neuropathy, general physical health deterioration, or any combination thereof. In some embodiments, the one or more adverse events is a grade 1 or greater adverse event. In some embodiments, the one or more adverse events is a grade 2 or greater adverse event. In some embodiments, the one or more adverse events is a grade 3 or greater adverse event. In some embodiments, the one or more adverse events is a grade 1 adverse event. In some embodiments, the one or more adverse events is a grade 2 adverse event. In some embodiments, the one or more adverse events is a grade 3 adverse event. In some embodiments, the one or more adverse events is a grade 4 adverse event. In some embodiments, the one or more adverse events is a serious adverse event. In some embodiments, the one or more adverse events is conjunctivitis, conjunctival ulceration, and/or keratitis and the additional therapeutic agent is a preservative-free lubricating eye drop, an ocular vasoconstrictor, an antibiotic, a steroid eye drop, or any combination thereof. In some embodiments, the one or more adverse events is conjunctivitis, conjunctival ulceration, and keratitis and the additional therapeutic agent is a preservative-free lubricating eye drop, an ocular vasoconstrictor, an antibiotic, a steroid eye drop, or any combination thereof. In some embodiments, the one or more adverse events is conjunctivitis and keratitis and the additional therapeutic agent is a preservative-free lubricating eye drop, an ocular vasoconstrictor, an antibiotic, a steroid eye drop, or any combination thereof. In some embodiments, the one or more adverse events is conjunctivitis and the additional therapeutic agent is a preservative-free lubricating eye drop, an ocular vasoconstrictor, an antibiotic, a steroid eye drop, or any combination thereof. In some embodiments, the one or more adverse events is keratitis and the additional therapeutic agent is a preservative-free lubricating eye drop, an ocular vasoconstrictor, an antibiotic, a steroid eye drop, or any combination thereof. In some of any of the embodiments herein, the subject is administered a treatment with the additional therapeutic agent to eliminate or reduce the severity of the adverse event (e.g., conjunctivitis, conjunctival ulceration, and/or keratitis). In some embodiments, the treatment is eye cooling pads (e.g. THERA PEARL Eye Mask or similar). In some embodiments, the one or more adverse events is a recurrent infusion related reaction and the additional therapeutic agent is an antihistamine, acetaminophen and/or a corticosteroid. In some embodiments, the one or more adverse events is neutropenia and the additional therapeutic agent is growth factor support (G-CSF).

In one aspect, the subject treated with an anti-TF antibody-drug conjugates or antigen-binding fragments thereof described herein, such as e.g., tisotumab vedotin, is at risk of developing one or more adverse events. In some embodiments, the subject is administered an additional therapeutic agent to prevent the development of the adverse event or to reduce the severity of the adverse event. In some embodiments, the one or more adverse events the subject is at risk of developing is anemia, abdominal pain, hypokalemia, hyponatremia, epistaxis, fatigue, nausea, alopecia, conjunctivitis, constipation, decreased appetite, diarrhea, vomiting, peripheral neuropathy, general physical health deterioration, or any combination thereof. In some embodiments, the one or more adverse events is a grade 1 or greater adverse event. In some embodiments, the one or more adverse events is a grade 2 or greater adverse event. In some embodiments, the one or more adverse events is a grade 3 or greater adverse event. In some embodiments, the one or more adverse events is a grade 1 adverse event. In some embodiments, the one or more adverse events is a grade 2 adverse event. In some embodiments, the one or more adverse events is a grade 3 adverse event. In some embodiments, the one or more adverse events is a grade 4 adverse event. In some embodiments, the one or more adverse events is a serious adverse event. In some embodiments, the one or more adverse events is conjunctivitis, conjunctival ulceration, and/or keratitis and the additional therapeutic agent is a preservative-free lubricating eye drop, an ocular vasoconstrictor, an antibiotic, a steroid eye drop, or any combination thereof. In some embodiments, the one or more adverse events is conjunctivitis, conjunctival ulceration, and keratitis and the additional therapeutic agent is a preservative-free lubricating eye drop, an ocular vasoconstrictor, an antibiotic, a steroid eye drop, or any combination thereof. In some embodiments, the one or more adverse events is conjunctivitis and keratitis and the additional therapeutic agent is a preservative-free lubricating eye drop, an ocular vasoconstrictor, an antibiotic, a steroid eye drop, or any combination thereof. In some embodiments, the one or more adverse events is conjunctivitis and the additional therapeutic agent is a preservative-free lubricating eye drop, an ocular vasoconstrictor, an antibiotic, a steroid eye drop, or any combination thereof. In some embodiments, the one or more adverse events is keratitis and the additional therapeutic agent is a preservative-free lubricating eye drop, an ocular vasoconstrictor, an antibiotic, a steroid eye drop, or any combination thereof. In some of any of the embodiments herein, the subject is administered a treatment with the additional therapeutic agent to prevent the development of the adverse event or to reduce the severity of the adverse event (e.g., conjunctivitis, conjunctival ulceration, and/or keratitis). In some embodiments, the treatment is eye cooling pads (e.g. THERA PEARL Eye Mask or similar). In some embodiments, the one or more adverse events is a recurrent infusion related reaction and the additional therapeutic agent is an antihistamine, acetaminophen and/or a corticosteroid. In some embodiments, the one or more adverse events is neutropenia and the additional therapeutic agent is growth factor support (G-CSF).

### IV. Compositions

In some aspects, also provided herein are compositions (e.g., pharmaceutical compositions and therapeutic formulations) comprising any of the anti-TF antibody-drug conjugates or antigen-binding fragments thereof described herein, such as e.g., tisotumab vedotin.

Therapeutic formulations are prepared for storage by mixing the active ingredient having the desired degree of purity with optional pharmaceutically acceptable carriers, excipients or stabilizers (Remington: The Science and Practice of Pharmacy, 20th Ed., Lippincott Williams & Wiklins, Pub., Gennaro Ed., Philadelphia, Pa. 2000).

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers can be used to control the pH in a range which optimizes the therapeutic effectiveness, especially if stability is pH dependent. Buffers can be present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives can be added to prevent microbial growth, and are typically present in a range from about 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intramolecular interactions. Tonicity agents can be present in any amount between about 0.1% to about 25% by weight or between about 1% to about 5% by weight, taking into account the relative amounts of the other ingredients. In some embodiments, tonicity agents include polyhydric sugar alcohols, trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) can be present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml or about 0.07 mg/ml to about 0.2 mg/ml. In some embodiments, non-ionic surfactants are present in a range of about 0.001% to about 0.1% w/v or about 0.01% to about 0.1% w/v or about 0.01% to about 0.025% w/v.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN@-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

Formulations comprising an anti-TF antibody-conjugate described herein for use in methods of treatment provided herein are described in WO2015/075201. In some embodiments, an anti-TF antibody-drug conjugate described herein is in a formulation comprising the anti-TF antibody drug conjugate, histidine, sucrose, and D-mannitol, wherein the formulation has a pH of about 6.0. In some embodiments, an anti-TF antibody-drug conjugate described herein is in a formulation comprising the anti-TF antibody drug conjugate at a concentration of about 10 mg/ml, histidine at a concentration of about 30 mM, sucrose at a concentration of about 88 mM, D-mannitol at a concentration of about 165 mM, wherein the formulation has a pH of about 6.0. In some embodiments, an anti-TF antibody-drug conjugate described herein is in a formulation comprising the anti-TF antibody drug conjugate at a concentration of 10 mg/ml, histidine at a concentration of 30 mM, sucrose at a concentration of 88 mM, D-mannitol at a concentration of 165 mM, wherein the formulation has a pH of 6.0. In some embodiments, the formulation comprises tisotumab vedotin at a concentration of 10 mg/ml, histidine at a concentration of 30 mM, sucrose at a concentration of 88 mM, D-mannitol at a concentration of 165 mM, wherein the formulation has a pH of 6.0.

In some embodiments provided herein, a formulation comprising the anti-TF antibody-conjugate described herein does not comprise a surfactant (i.e., is free of surfactant).

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The invention provides compositions comprising a population of anti-TF antibody-drug conjugates or antigen-binding fragments thereof as described herein for use in a method of treating colorectal cancer, non-small cell lung cancer, pancreatic cancer, head and neck cancer bladder cancer, endometrial cancer, esophageal cancer or prostate cancer as described herein. In some aspects, provided herein are compositions comprising a population of antibody-drug conjugates, wherein the antibody-drug conjugates comprise a linker attached to MMAE, wherein the antibody-drug conjugate has the following structure:

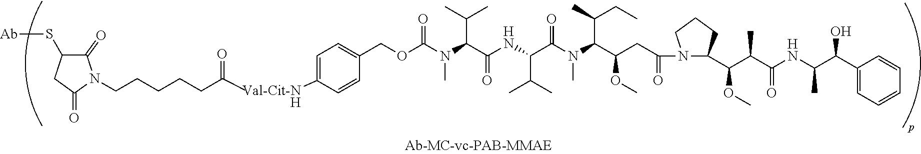

wherein p denotes a number from 1 to 8, e.g., 1, 2, 3, 4, 5, 6, 7 or 8, S represents a sulphydryl residue of the anti-TF antibody or antigen-binding fragment thereof, and Ab designates the anti-TF antibody or antigen-binding fragment thereof as described herein, such as tisotumab. In some embodiments, p denotes a number from 3 to 5. In some embodiments, the average value of p in the composition is about 4. In some embodiments, the population is a mixed population of antibody-drug conjugates in which p varies from 1 to 8 for each antibody-drug conjugate. In some embodiments, the population is a homogenous population of antibody-drug conjugates with each antibody-drug conjugate having the same value for p.

In some embodiments, a composition comprising an anti-TF antibody-drug conjugate as described herein, such as e.g., tisotumab vedotin, is coadministered with one or more additional therapeutic agents. In some embodiments the coadministration is simultaneous or sequential. In some embodiments, the anti-TF antibody-drug conjugate as described herein is administered simultaneously with the one or more additional therapeutic agents. In some embodiments, simultaneous means that the anti-TF antibody-drug conjugate and the one or more additional therapeutic agents are administered to the subject less than about one hour apart, such as less than about 30 minutes apart, less than about 15 minutes apart, less than about 10 minutes apart or less than about 5 minutes apart. In some embodiments, simultaneous means that the anti-TF antibody-drug conjugate and the one or more additional therapeutic agents are administered to the subject less than one hour apart, such as less than 30 minutes apart, less than 15 minutes apart, less than 10 minutes apart or less than 5 minutes apart. In some embodiments, the anti-TF antibody-drug conjugate is administered sequentially with the one or more additional therapeutic agents. In some embodiments, sequential administration means that the anti-TF antibody-drug conjugate and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart.

In some embodiments, a composition comprising an anti-TF antibody-drug conjugate as described herein, such as e.g., tisotumab vedotin, is coadministered with one or more therapeutic agents to eliminate or reduce the severity of one or more adverse events. In some embodiments the coadministration is simultaneous or sequential. In some embodiments, the anti-TF antibody-drug conjugate is administered simultaneously with the one or more therapeutic agents to eliminate or reduce the severity of one or more adverse events. In some embodiments, simultaneous means that the anti-TF antibody-drug conjugate and the one or more therapeutic agents to eliminate or reduce the severity of one or more adverse events are administered to the subject less than about one hour apart, such as less than about 30 minutes apart, less than about 15 minutes apart, less than about 10 minutes apart or less than about 5 minutes apart. In some embodiments, simultaneous means that the anti-TF antibody-drug conjugate and the one or more therapeutic agents to eliminate or reduce the severity of one or more adverse events are administered to the subject less than one hour apart, such as less than 30 minutes apart, less than 15 minutes apart, less than 10 minutes apart or less than 5 minutes apart. In some embodiments, the anti-TF antibody-drug conjugate is administered sequentially with the one or more therapeutic agents to eliminate or reduce the severity of one or more adverse events. In some embodiments, sequential administration means that the anti-TF antibody-drug conjugate and the one or more therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, the anti-TF antibody-drug conjugate is administered prior to the one or more therapeutic agents to eliminate or reduce the severity of one or more adverse events. In some embodiments, the one or more therapeutic agents to eliminate or reduce the severity of one or more adverse events is administered prior to the anti-TF antibody-drug conjugate.

### V. Articles of Manufacture and Kits

In another aspect, an article of manufacture or kit is provided which comprises an anti-TF antibody-drug conjugate described herein, such as e.g., tisotumab vedotin. The article of manufacture or kit may further comprise instructions for use of the anti-TF antibody-drug conjugate in the methods of the invention. Thus, in certain embodiments, the article of manufacture or kit comprises instructions for the use of an anti-TF antibody-drug conjugate in methods for treating cancer (e.g., colorectal cancer, non-small cell lung cancer, pancreatic cancer, head and neck cancer, bladder cancer, endometrial cancer, esophageal cancer or prostate cancer) in a subject comprising administering to the subject an effective amount of an anti-TF antibody-drug conjugate. In some embodiments, the cancer is colorectal cancer as described herein. In some embodiments, the cancer is non-small cell lung cancer as described herein. In some embodiments, the cancer is pancreatic cancer as described herein. In some embodiments, the cancer is head and neck cancer as described herein. In some embodiments, the cancer is bladder cancer as described herein. In some embodiments, the cancer is endometrial cancer as described herein. In some embodiments, the cancer is esophageal cancer as described herein. In some embodiments, the cancer is prostate cancer as described herein. In some embodiments, the subject is a human.

The article of manufacture or kit may further comprise a container. Suitable containers include, for example, bottles, vials (e.g., dual chamber vials), syringes (such as single or dual chamber syringes) and test tubes. In some embodiments, the container is a vial. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous (e.g., intravenous infusion), or other modes of administration for treating colorectal cancer, non-small cell lung cancer, pancreatic cancer, head and neck cancer, bladder cancer, endometrial cancer, esophageal cancer or prostate cancer as described herein in a subject. The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

The article of manufacture or kit herein optionally further comprises a container comprising a second medicament, wherein the anti-TF antibody-drug conjugate is a first medicament, and which article or kit further comprises instructions on the label or package insert for treating the subject with the second medicament, in an effective amount. In some embodiments, the label or package insert indicates that the first and second medicaments are to be administered sequentially or simultaneously, as described herein. In some embodiments, the label or package insert indicates that the first medicament is to be administered prior to the administration of the second medicament. In some embodiments, the label or package insert indicates that second medicament is to be administered prior to the first medicament.

The article of manufacture or kit herein optionally further comprises a container comprising a second medicament, wherein the second medicament is for eliminating or reducing the severity of one or more adverse events, wherein the anti-TF antibody-drug conjugate is a first medicament, and which article or kit further comprises instructions on the label or package insert for treating the subject with the second medicament, in an effective amount. In some embodiments, the label or package insert indicates that the first and second medicaments are to be administered sequentially or simultaneously, as described herein. In some embodiments, the label or package insert indicates that the first medicament is to be administered prior to the administration of the second medicament. In some embodiments, the label or package insert indicates that second medicament is to be administered prior to the first medicament.

In some embodiments, the anti-TF antibody-drug conjugate is present in the container as a lyophilized powder. In some embodiments, the lyophilized powder is in a hermetically sealed container, such as a vial, an ampoule or sachette, indicating the quantity of the active agent. Where the pharmaceutical is administered by injection, an ampoule of sterile water for injection or saline can be, for example, provided, optionally as part of the kit, so that the ingredients can be mixed prior to administration. Such kits can further include, if desired, one or more of various conventional pharmaceutical components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components can also be included in the kit.

### VI. Exemplary Embodiments

Among the embodiments provided herein are:

1. A method of treating cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof, wherein the antibody-drug conjugate is administered at a dose ranging from about 1.5 mg/kg to about 2.1 mg/kg, and wherein the cancer is selected from the group consisting of colorectal cancer, non-small cell lung cancer, pancreatic cancer, head and neck cancer, bladder cancer, endometrial cancer, esophageal cancer and prostate cancer.
2. The method of embodiment 1, wherein the dose is about 2.0 mg/kg.
3. The method of embodiment 1, wherein the dose is 2.0 mg/kg.
4. The method of any one of embodiments 1-3, wherein the antibody-drug conjugate is administered once about every 1 week, 2 weeks, 3 weeks or 4 weeks.
5. The method of any one of embodiments 1-4, wherein the antibody-drug conjugate is administered once about every 3 weeks.
6. The method of any one of embodiments 1-5, wherein the subject has been previously treated with one or more therapeutic agents and did not respond to the treatment, wherein the one or more therapeutic agents is not the antibody-drug conjugate.
7. The method of any one of embodiments 1-5, wherein the subject has been previously treated with one or more therapeutic agents and relapsed after the treatment, wherein the one or more therapeutic agents is not the antibody-drug conjugate.
8. The method of any one of embodiments 1-5, wherein the subject has been previously treated with one or more therapeutic agents and has experienced disease progression during treatment, wherein the one or more therapeutic agents is not the antibody-drug conjugate.
9. The method of any one of embodiments 1-8, wherein the cancer is colorectal cancer.
10. The method of embodiment 9, wherein the subject received prior systemic therapy and experienced disease progression on or after the systemic therapy.
11. The method of embodiment 10, wherein the subject received 1, 2 or 3 rounds of prior systemic therapy.
12. The method of any one of embodiments 9-11, wherein the colorectal cancer is non-operable.
13. The method of any one of embodiments 9-12, wherein the subject has been previously treated with one or more agents selected from the group consisting of fluoropyrimidine, oxaliplatin, irinotecan and bevacizumab.
14. The method of any one of embodiments 9-13, wherein the subject has been previously treated with one or more agents selected from the group consisting of cetuximab, panitumab and a checkpoint inhibitor.
15. The method of any one of embodiments 1-8, wherein the cancer is non-small cell lung cancer.
16. The method of embodiment 15, wherein the non-small cell lung cancer is squamous cell carcinoma.
17. The method of embodiment 15 or embodiment 16, wherein the non-small cell lung cancer has predominant squamous histology.
18. The method of embodiment 17, wherein greater than 85% of the non-small cell lung cancer cells have squamous histology.
19. The method of embodiment 15, wherein the non-small cell lung cancer is adenocarcinoma.
20. The method of any one of embodiments 15-19, wherein the subject received prior systemic therapy and experienced disease progression on or after the systemic therapy.
21. The method of embodiment 20, wherein the subject received 1 or 2 rounds of prior systemic therapy.
22. The method of any one of embodiments 15-21, wherein the subject has been previously treated with one or more agents selected from the group consisting of a platinum-based therapy and a checkpoint inhibitor.
23. The method of any one of embodiments 1-8, wherein the cancer is pancreatic cancer.
24. The method of embodiment 23, wherein the pancreatic cancer is exocrine pancreatic adenocarcinoma.
25. The method of embodiment 23 or embodiment 24, wherein the pancreatic cancer has predominant adenocarcinoma histology.
26. The method of embodiment 25, wherein greater than 85% of the pancreatic cancer cells have adenocarcinoma histology.
27. The method of any one of embodiments 23-26, wherein the subject received prior systemic therapy and experienced disease progression on or after the systemic therapy.
28. The method of embodiment 27, wherein the subject received 1 round of prior systemic therapy.
29. The method of any one of embodiments 23-28, wherein the subject has been previously treated with one or more agents selected from the group consisting of gemcitabine and 5-fluorouracil.
30. The method of any one of embodiments 23-29, wherein the pancreatic cancer is not resectable.
31. The method of any one of embodiments 1-8, wherein the cancer is head and neck cancer.
32. The method of embodiment 31, wherein the head and neck cancer is squamous cell carcinoma.
33. The method of embodiment 31 or embodiment 32, wherein the subject received prior systemic therapy and experienced disease progression on or after the systemic therapy.
34. The method of embodiment 33, wherein, the subject received 1 or 2 rounds of prior systemic therapy.
35. The method of any one of embodiments 31-34, wherein the subject has been previously treated with one or more agents selected from the group consisting of a platinum-based therapy and a checkpoint inhibitor.
36. The method of anyone of embodiments 31-35, wherein the subject has been previously treated with an anti-epithelial growth factor receptor therapy.
37. The method of any one of embodiments 1-8, wherein the cancer is bladder cancer.
38. The method of embodiment 37, wherein the subject received prior systemic therapy and experienced disease progression on or after the systemic therapy.
39. The method of embodiment 38, wherein the subject received 1, 2 or 3 rounds of prior systemic therapy.
40. The method of any one of embodiments 37-39, wherein the subject has been previously treated with a platinum-based therapy.
41. The method of any one of embodiments 37-40, wherein the subject has previously undergone surgery or radiation therapy for the bladder cancer.
42. The method of any one of embodiments 1-8, wherein the cancer is endometrial cancer.
43. The method of embodiment 42, wherein the subject received prior systemic therapy and experienced disease progression on or after the systemic therapy.
44. The method of embodiment 43, wherein the subject received 1, 2 or 3 rounds of prior systemic therapy.
45. The method of any one of embodiments 42-44, wherein the subject has been previously treated with one or more agents selected from the group consisting of a platinum-based therapy, hormone therapy, and a checkpoint inhibitor.
46. The method of any one of embodiments 42-45, wherein the subject has previously been treated with doxorubicin.
47. The method of any one of embodiments 42-46, wherein the subject has previously been treated with paclitaxel.
48. The method of any one of embodiments 42-47, wherein the subject has previously undergone surgery or radiation therapy for the endometrial cancer.
49. The method of any one of embodiments 1-8, wherein the cancer is esophageal cancer.
50. The method of embodiment 49, wherein the subject received prior systemic therapy and experienced disease progression on or after the systemic therapy.
51. The method of embodiment 50, wherein the subject received 1, 2 or 3 rounds of prior systemic therapy.
52. The method of anyone of embodiments 49-51, wherein the subject has been previously treated with one or more agents selected from the group consisting of a platinum-based therapy and a checkpoint inhibitor.
53. The method of any one of embodiments 49-52, wherein the subject has been previously treated with one or more agents selected from the group consisting of ramucirumab, paclitaxel, 5-fluorouracil, docetaxel, irinotecan, capecitabine and trastuzumab.
54. The method of any one of embodiments 49-53, wherein the subject has previously undergone surgery, radiation therapy or endoscopic mucosal resection for the esophageal cancer.
55. The method of any one of embodiments 1-8, wherein the cancer is prostate cancer.
56. The method of embodiment 55, wherein the subject received prior systemic therapy and experienced disease progression on or after the systemic therapy.
57. The method of embodiment 56, wherein the subject received 1, 2 or 3 rounds of prior systemic therapy.
58. The method of any one of embodiments 55-57, wherein the prostate cancer is castration-resistant prostate cancer.
59. The method of any one of embodiments 55-58, wherein the subject experienced bone metastases.
60. The method of any one of embodiments 55-59, wherein the subject has been previously treated with one or more agents selected from the group consisting of androgen deprivation therapy, a luteinizing hormone-releasing hormone agonist, a luteinizing hormone-releasing hormone antagonist, a CYP17 inhibitor, and an anti-androgen.
61. The method of any one of embodiments 55-60, wherein the subject has been previously treated with one or more agents selected from the group consisting of docetaxel, prednisone and cabazitaxel.
62. The method of anyone of embodiments 55-61, wherein the subject has previously undergone surgery or radiation therapy for the prostate cancer.
63. The method of any one of embodiments 1-62, wherein the cancer is an advanced stage cancer.
64. The method of embodiment 63, wherein the advanced stage cancer is a stage 3 or stage 4 cancer.
65. The method of embodiment 63 or 64, wherein the advanced stage cancer is metastatic cancer.
66. The method of any one of embodiments 1-65, wherein the cancer is recurrent cancer.
67. The method of any one of embodiments 1-66, wherein the subject received prior treatment with standard of care therapy for the cancer and failed the prior treatment.
68. The method of any one of embodiments 1-67, wherein the monomethyl auristatin is monomethyl auristatin E (MMAE).
69. The method of any one of embodiments 1-68, wherein the anti-TF antibody or antigen-binding fragment thereof of the antibody-drug conjugate is a monoclonal antibody or a monoclonal antigen-binding fragment thereof.
70. The method of any one of embodiments 1-69, wherein the anti-TF antibody or antigen-binding fragment thereof of the antibody-drug conjugate comprises a heavy chain variable region and a light chain variable region, wherein the heavy chain variable region comprises:
(i) a CDR-H1 comprising the amino acid sequence of SEQ ID NO:1;

(ii) a CDR-H2 comprising the amino acid sequence of SEQ ID NO:2; and

(iii) a CDR-H3 comprising the amino acid sequence of SEQ ID NO:3; and wherein the light chain variable region comprises:

(i) a CDR-L1 comprising the amino acid sequence of SEQ ID NO4;

(ii) a CDR-L2 comprising the amino acid sequence of SEQ ID NO:5; and

(iii) a CDR-L3 comprising the amino acid sequence of SEQ ID NO:6.

71. The method of any one of embodiments 1-70, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.
72. The method of any one of embodiments 1-71, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
73. The method of any one of embodiments 1-72, wherein the anti-TF antibody of the antibody-drug conjugate is tisotumab.
74. The method of any one of embodiments 1-73, wherein the antibody-drug conjugate further comprises a linker between the anti-TF antibody or antigen-binding fragment thereof and the monomethyl auristatin.
75. The method of embodiment 74, wherein the linker is a cleavable peptide linker.
76. The method of embodiment 75, wherein the cleavable peptide linker has a formula: -MC-vc-PAB-, wherein:

a) MC is:

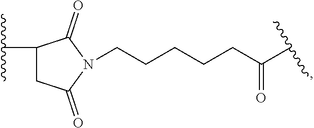 [SMILES: *C(=O)CCCCCN1C(=O)CC(*)C1=O]

b) vc is the dipeptide valine-citrulline, and

c) PAB is:

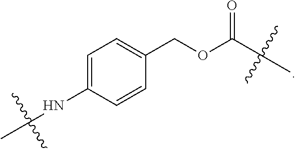 [SMILES: *Nc1ccc(COC(*)=O)cc1]

77. The method of any one of embodiments 74-76, wherein the linker is attached to sulphydryl residues of the anti-TF antibody obtained by partial reduction or full reduction of the anti-TF antibody or antigen-binding fragment thereof.
78. The method of embodiment 77, wherein the linker is attached to monomethyl auristatin E (MMAE), wherein the antibody-drug conjugate has the following structure:

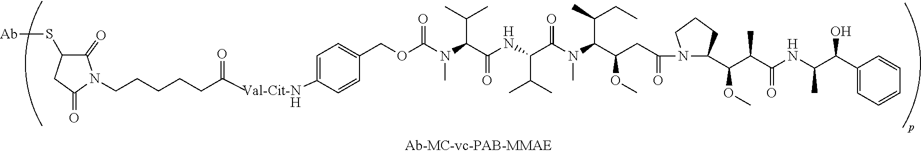

wherein p denotes a number from 1 to 8, S represents a sulphydryl residue of the anti-TF antibody, and Ab designates the anti-TF antibody or antigen-binding fragment thereof.
79. The method of embodiment 78, wherein the average value of p in a population of the antibody-drug conjugates is about 4.
80. The method of any one of embodiments 1-79, wherein the antibody-drug conjugate is tisotumab vedotin.
81. The method of any one of embodiments 1-80, wherein the route of administration for the antibody-drug conjugate is intravenous.
82. The method of any one of embodiments 1-81, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express TF.
83. The method of any one of embodiments 1-82, wherein one or more therapeutic effects in the subject is improved after administration of the antibody-drug conjugate relative to a baseline.
84. The method of embodiment 83, wherein the one or more therapeutic effects is selected from the group consisting of: size of a tumor derived from the cancer, objective response rate, duration of response, time to response, progression free survival, overall survival and prostate specific antigen (PSA) level.
85. The method of any one of embodiments 55-62, wherein the subject exhibits a reduction in PSA level in a blood sample from the subject by at least about 5%, at least about 10%, at least about 15%, at least about 20%, at least about 25%, at least about 30%, at least about 35%, at least about 40%, at least about 45%, at least about 50%, at least about 60%, at least about 70%, or at least about 80% relative to the PSA level in a blood sample obtained from the subject before administration of the antibody-drug conjugate.
86. The method of any one of embodiments 1-85, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate.
87. The method of any one of embodiments 1-86, wherein the objective response rate is at least about 20%, at least about 25%, at least about 30%, at least about 35%, at least about 40%, at least about 45%, at least about 50%, at least about 60%, at least about 70%, or at least about 80%.
88. The method of any one of embodiments 1-87, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
89. The method of any one of embodiments 1-88, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
90. The method of any one of embodiments 1-89, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
91. The method of any one of embodiments 1-90, wherein the subject has one or more adverse events and is further administered an additional therapeutic agent to eliminate or reduce the severity of the one or more adverse events.
92. The method of any one of embodiments 1-90, wherein the subject is at risk of developing one or more adverse events and is further administered an additional therapeutic agent to prevent or reduce the severity of the one or more adverse events.
93. The method of embodiment 91 or embodiment 92, wherein the one or more adverse events is anemia, abdominal pain, hypokalemia, hyponatremia, epistaxis, fatigue, nausea, alopecia, conjunctivitis, constipation, decreased appetite, diarrhea, vomiting, peripheral neuropathy, or general physical health deterioration.
94. The method of embodiment 91 or embodiment 92, wherein the one or more adverse events is a grade 3 or greater adverse event.
95. The method of embodiment 91 or embodiment 92, wherein the one or more adverse events is a serious adverse event.
96. The method of embodiment 91 or embodiment 92, wherein the one or more adverse events is conjunctivitis and/or keratitis and the additional agent is a preservative-free lubricating eye drop, an ocular vasoconstrictor and/or a steroid eye drop.
97. The method of any one of embodiments 1-96, wherein the antibody-drug conjugate is administered as a monotherapy.
98. The method of any one of embodiments 1-97, wherein the subject is a human.
99. The method of any one of embodiments 1-98, wherein the antibody-drug conjugate is in a pharmaceutical composition comprising the antibody-drug conjugate and a pharmaceutical acceptable carrier.
100. A kit comprising:

(a) a dosage ranging from about 0.9 mg/kg to about 2.1 mg/kg of an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof; and

(b) instructions for using the antibody drug conjugate according to the method of any one of embodiments 1-99.

101. An antibody-drug conjugate that binds to TF for use in the treatment of cancer in a subject, wherein the antibody-drug conjugate comprises an anti-TF antibody or an antigen-binding fragment thereof conjugated to a monomethyl auristatin or a functional analog thereof or a functional derivative thereof, wherein the antibody-drug conjugate is administered to the subject at a dose ranging from about 0.9 mg/kg to about 2.1 mg/kg, and wherein the cancer is selected from the group consisting of colorectal cancer, non-small cell lung cancer, pancreatic cancer, head and neck cancer, bladder cancer, endometrial cancer, esophageal cancer and prostate cancer.
102. The antibody-drug conjugate for use of embodiment 101, wherein the dose is about 2.0 mg/kg.
103. The antibody-drug conjugate for use of embodiment 101, wherein the dose is 2.0 mg/kg.
104. The antibody-drug conjugate for use of any one of embodiments 101-103, wherein the antibody-drug conjugate is administered once about every 1 week, 2 weeks, 3 weeks or 4 weeks.
105. The antibody-drug conjugate for use of any one of embodiments 101-104, wherein the antibody-drug conjugate is administered once about every 3 weeks.
106. The antibody-drug conjugate for use of any one of embodiments 101-105, wherein the subject has been previously treated with one or more therapeutic agents and did not respond to the treatment, wherein the one or more therapeutic agents is not the antibody-drug conjugate.
107. The antibody-drug conjugate for use of any one of embodiments 101-105, wherein the subject has been previously treated with one or more therapeutic agents and relapsed after the treatment, wherein the one or more therapeutic agents is not the antibody-drug conjugate.
108. The antibody-drug conjugate for use of any one of embodiments 101-105, wherein the subject has been previously treated with one or more therapeutic agents and has experienced disease progression during treatment, wherein the one or more therapeutic agents is not the antibody-drug conjugate.
109. The antibody-drug conjugate for use of any one of embodiments 101-108, wherein the cancer is colorectal cancer.
110. The antibody-drug conjugate for use of embodiment 109, wherein the subject received prior systemic therapy and experienced disease progression on or after the systemic therapy.
111. The antibody-drug conjugate for use of embodiment 110, wherein the subject received 1, 2 or 3 rounds of prior systemic therapy.
112. The antibody-drug conjugate for use of any one of embodiments 109-111, wherein the colorectal cancer is non-operable.
113. The antibody-drug conjugate for use of any one of embodiments 109-112, wherein the subject has been previously treated with one or more agents selected from the group consisting of fluoropyrimidine, oxaliplatin, irinotecan and bevacizumab.
114. The antibody-drug conjugate for use of any one of embodiments 109-113, wherein the subject has been previously treated with one or more agents selected from the group consisting of cetuximab, panitumab and a checkpoint inhibitor.
115. The antibody-drug conjugate for use of anyone of embodiments 101-108, wherein the cancer is non-small cell lung cancer.
116. The antibody-drug conjugate for use of embodiment 115, wherein the non-small cell lung cancer is squamous cell carcinoma.
117. The antibody-drug conjugate for use of embodiment 115 or embodiment 116, wherein the non-small cell lung cancer has predominant squamous histology.
118. The antibody-drug conjugate for use of embodiment 117, wherein greater than 85% of the non-small cell lung cancer cells have squamous histology.
119. The antibody-drug conjugate for use of embodiment 115, wherein the non-small cell lung cancer is adenocarcinoma.
120. The antibody-drug conjugate for use of any one of embodiments 115-119, wherein the subject received prior systemic therapy and experienced disease progression on or after the systemic therapy.
121. The antibody-drug conjugate for use of embodiment 120, wherein the subject received 1 or 2 rounds of prior systemic therapy.
122. The antibody-drug conjugate for use of any one of embodiments 115-121, wherein the subject has been previously treated with one or more agents selected from the group consisting of a platinum-based therapy and a checkpoint inhibitor.
123. The antibody-drug conjugate for use of any one of embodiments 101-108, wherein the cancer is pancreatic cancer.
124. The antibody-drug conjugate for use of embodiment 123, wherein the pancreatic cancer is exocrine pancreatic adenocarcinoma.
125. The antibody-drug conjugate for use of embodiment 123 or embodiment 124, wherein the pancreatic cancer has predominant adenocarcinoma histology.
126. The antibody-drug conjugate for use of embodiment 125, wherein greater than 85% of the pancreatic cancer cells have adenocarcinoma histology.
127. The antibody-drug conjugate for use of any one of embodiments 123-126, wherein the subject received prior systemic therapy and experienced disease progression on or after the systemic therapy.
128. The method of embodiment 127, wherein the subject received 1 round of prior systemic therapy.
129. The antibody-drug conjugate for use of any one of embodiments 123-128, wherein the subject has been previously treated with one or more agents selected from the group consisting of gemcitabine and 5-fluorouracil.
130. The antibody-drug conjugate for use of any one of embodiments 123-129, wherein the pancreatic cancer is not resectable.
131. The antibody-drug conjugate for use of anyone of embodiments 101-108, wherein the cancer is head and neck cancer.
132. The antibody-drug conjugate for use of embodiment 131, wherein the head and neck cancer is squamous cell carcinoma.
133. The antibody-drug conjugate for use of embodiment 131 or embodiment 132, wherein the subject received prior systemic therapy and experienced disease progression on or after the systemic therapy.
134. The antibody-drug conjugate for use of embodiment 133, wherein, the subject received 1 or 2 rounds of prior systemic therapy.
135. The antibody-drug conjugate for use of any one of embodiments 131-134, wherein the subject has been previously treated with one or more agents selected from the group consisting of a platinum-based therapy and a checkpoint inhibitor.
136. The antibody-drug conjugate for use of any one of embodiments 131-135, wherein the subject has been previously treated with an anti-epithelial growth factor receptor therapy.
137. The antibody-drug conjugate for use of any one of embodiments 101-108, wherein the canceris bladder cancer.
138. The antibody-drug conjugate for use of embodiment 137, wherein the subject received prior systemic therapy and experienced disease progression on or after the systemic therapy.
139. The antibody-drug conjugate for use of embodiment 138, wherein the subject received 1, 2 or 3 rounds of prior systemic therapy.
140. The antibody-drug conjugate for use of any one of embodiments 137-139, wherein the subject has been previously treated with a platinum-based therapy.
141. The antibody-drug conjugate for use of any one of embodiments 137-140, wherein the subject has previously undergone surgery or radiation therapy for the bladder cancer.
142. The antibody-drug conjugate for use of any one of embodiments 101-108, wherein the cancer is endometrial cancer.
143. The antibody-drug conjugate for use of embodiment 142, wherein the subject received prior systemic therapy and experienced disease progression on or after the systemic therapy.
144. The antibody-drug conjugate for use of embodiment 143, wherein the subject received 1, 2 or 3 rounds of prior systemic therapy.
145. The antibody-drug conjugate for use of any one of embodiments 142-144, wherein the subject has been previously treated with one or more agents selected from the group consisting of a platinum-based therapy, hormone therapy, and a checkpoint inhibitor.
146. The antibody-drug conjugate for use of any one of embodiments 142-145, wherein the subject has previously been treated with doxorubicin.
147. The antibody-drug conjugate for use of any one of embodiments 142-146, wherein the subject has previously been treated with paclitaxel.
148. The antibody-drug conjugate for use of any one of embodiments 142-147, wherein the subject has previously undergone surgery or radiation therapy for the endometrial cancer.
149. The antibody-drug conjugate for use of any one of embodiments 101-108, wherein the cancer is esophageal cancer.
150. The antibody-drug conjugate for use of embodiment 149, wherein the subject received prior systemic therapy and experienced disease progression on or after the systemic therapy.
151. The antibody-drug conjugate for use of embodiment 150, wherein the subject received 1, 2 or 3 rounds of prior systemic therapy.
152. The antibody-drug conjugate for use of any one of embodiments 149-151, wherein the subject has been previously treated with one or more agents selected from the group consisting of a platinum-based therapy and a checkpoint inhibitor.
153. The antibody-drug conjugate for use of any one of embodiments 149-152, wherein the subject has been previously treated with one or more agents selected from the group consisting of ramucirumab, paclitaxel, 5-fluorouracil, docetaxel, irinotecan, capecitabine and trastuzumab.
154. The antibody-drug conjugate for use of any one of embodiments 149-153, wherein the subject has previously undergone surgery, radiation therapy or endoscopic mucosal resection for the esophageal cancer.
155. The antibody-drug conjugate for use of any one of embodiments 101-108, wherein the cancer is prostate cancer.
156. The antibody-drug conjugate for use of embodiment 155, wherein the subject received prior systemic therapy and experienced disease progression on or after the systemic therapy.
157. The antibody-drug conjugate for use of embodiment 156, wherein the subject received 1, 2 or 3 rounds of prior systemic therapy.
158. The antibody-drug conjugate for use of any one of embodiments 155-157, wherein the prostate cancer is castration-resistant prostate cancer.
159. The antibody-drug conjugate for use of any one of embodiments 155-158, wherein the subject experienced bone metastases.
160. The antibody-drug conjugate for use of any one of embodiments 155-159, wherein the subject has been previously treated with one or more agents selected from the group consisting of androgen deprivation therapy, a luteinizing hormone-releasing hormone agonist, a luteinizing hormone-releasing hormone antagonist, a CYP17 inhibitor, and an anti-androgen.
161. The antibody-drug conjugate for use of any one of embodiments 155-160, wherein the subject has been previously treated with one or more agents selected from the group consisting of docetaxel, prednisone and cabazitaxel.
162. The antibody-drug conjugate for use of any one of embodiments 155-161, wherein the subject has previously undergone surgery or radiation therapy for the prostate cancer.
163. The antibody-drug conjugate for use of any one of embodiments 101-162, wherein the cancer is an advanced stage cancer.
164. The antibody-drug conjugate for use of embodiment 163, wherein the advanced stage cancer is a stage 3 or stage 4 cancer.
165. The antibody-drug conjugate for use of embodiment 163 or 164, wherein the advanced stage cancer is metastatic cancer.
166. The antibody-drug conjugate for use of any one of embodiments 101-165, wherein the cancer is recurrent cancer.
167. The antibody-drug conjugate for use of any one of embodiments 101-166, wherein the subject received prior treatment with standard of care therapy for the cancer and failed the prior treatment.
168. The antibody-drug conjugate for use of any one of embodiments 101-167, wherein the monomethyl auristatin is monomethyl auristatin E (MMAE).
169. The antibody-drug conjugate for use of any one of embodiments 101-168, wherein the anti-TF antibody or antigen-binding fragment thereof of the antibody-drug conjugate is a monoclonal antibody or a monoclonal antigen-binding fragment thereof.
170. The antibody-drug conjugate for use of any one of embodiments 101-169, wherein the anti-TF antibody or antigen-binding fragment thereof of the antibody-drug conjugate comprises a heavy chain variable region and a light chain variable region, wherein the heavy chain variable region comprises:

(i) a CDR-H1 comprising the amino acid sequence of SEQ ID NO:1;

(ii) a CDR-H2 comprising the amino acid sequence of SEQ ID NO:2; and

(iii) a CDR-H3 comprising the amino acid sequence of SEQ ID NO:3; and wherein the light chain variable region comprises:

(i) a CDR-L1 comprising the amino acid sequence of SEQ ID NO4;

(ii) a CDR-L2 comprising the amino acid sequence of SEQ ID NO:5; and

(iii) a CDR-L3 comprising the amino acid sequence of SEQ ID NO:6.

171. The antibody-drug conjugate for use of any one of embodiments 101-170, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.
172. The antibody-drug conjugate for use of any one of embodiments 101-171, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
173. The antibody-drug conjugate for use of any one of embodiments 101-172, wherein the anti-TF antibody of the antibody-drug conjugate is tisotumab.
174. The antibody-drug conjugate for use of any one of embodiments 101-173, wherein the antibody-drug conjugate further comprises a linker between the anti-TF antibody or antigen-binding fragment thereof and the monomethyl auristatin.
175. The antibody-drug conjugate for use of embodiment 174, wherein the linker is a cleavable peptide linker.
176. The antibody-drug conjugate for use of embodiment 175, wherein the cleavable peptide linker has a formula: -MC-vc-PAB-, wherein:

a) MC is:

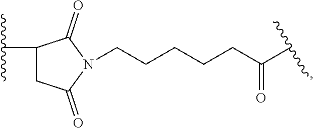 [SMILES: *C(=O)CCCCCN1C(=O)CC(*)C1=O]

b) vc is the dipeptide valine-citrulline, and

c) PAB is:

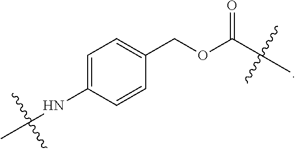 [SMILES: *Nc1ccc(COC(*)=O)cc1]

177. The antibody-drug conjugate for use of any one of embodiments 174-176, wherein the linker is attached to sulphydryl residues of the anti-TF antibody obtained by partial reduction or full reduction of the anti-TF antibody or antigen-binding fragment thereof.
178. The antibody-drug conjugate for use of embodiment 177, wherein the linker is attached to monomethyl auristatin E (MMAE), wherein the antibody-drug conjugate has the following structure:

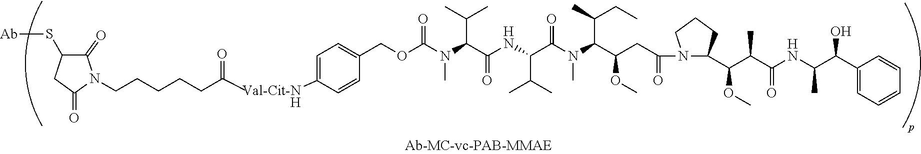

wherein p denotes a number from 1 to 8, S represents a sulphydryl residue of the anti-TF antibody, and Ab designates the anti-TF antibody or antigen-binding fragment thereof.
179. The antibody-drug conjugate for use of embodiment 178, wherein the average value of p in a population of the antibody-drug conjugates is about 4.
180. The antibody-drug conjugate for use of any one of embodiments 101-179, wherein the antibody-drug conjugate is tisotumab vedotin.
181. The antibody-drug conjugate for use of anyone of embodiments 101-180, wherein the route of administration for the antibody-drug conjugate is intravenous.
182. The antibody-drug conjugate for use of anyone of embodiments 101-181, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express TF.
183. The antibody-drug conjugate for use of anyone of embodiments 101-182, wherein one or more therapeutic effects in the subject is improved after administration of the antibody-drug conjugate relative to a baseline.
184. The antibody-drug conjugate for use of embodiment 183, wherein the one or more therapeutic effects is selected from the group consisting of: size of a tumor derived from the cancer, objective response rate, duration of response, time to response, progression free survival, overall survival and prostate specific antigen (PSA) level.
185. The antibody-drug conjugate for use of any one of embodiments 155-162, wherein the subject exhibits a reduction in PSA level in a blood sample from the subject by at least about 5%, at least about 10%, at least about 15%, at least about 20%, at least about 25%, at least about 30%, at least about 35%, at least about 40%, at least about 45%, at least about 50%, at least about 60%, at least about 70%, or at least about 80% relative to the PSA level in a blood sample obtained from the subject before administration of the antibody-drug conjugate.
186. The antibody-drug conjugate for use of anyone of embodiments 101-185, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate.
187. The antibody-drug conjugate for use of anyone of embodiments 101-186, wherein the objective response rate is at least about 20%, at least about 25%, at least about 30%, at least about 35%, at least about 40%, at least about 45%, at least about 50%, at least about 60%, at least about 70%, or at least about 80%.
188. The antibody-drug conjugate for use of anyone of embodiments 101-187, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
189. The antibody-drug conjugate for use of anyone of embodiments 101-188, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
190. The antibody-drug conjugate for use of anyone of embodiments 101-189, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
191. The antibody-drug conjugate for use of any one of embodiments 101-190, wherein the subject has one or more adverse events and is further administered an additional therapeutic agent to eliminate or reduce the severity of the one or more adverse events.
192. The antibody-drug conjugate for use of any one of embodiments 101-190, wherein the subject is at risk of developing one or more adverse events and is further administered an additional therapeutic agent to prevent or reduce the severity of the one or more adverse events.
193. The antibody-drug conjugate for use of embodiment 191 or embodiment 192, wherein the one or more adverse events is anemia, abdominal pain, hypokalemia, hyponatremia, epistaxis, fatigue, nausea, alopecia, conjunctivitis, constipation, decreased appetite, diarrhea, vomiting, peripheral neuropathy, or general physical health deterioration.
194. The antibody-drug conjugate for use of embodiment 191 or embodiment 192, wherein the one or more adverse events is a grade 3 or greater adverse event.
195. The antibody-drug conjugate for use of embodiment 191 or embodiment 192, wherein the one or more adverse events is a serious adverse event.
196. The antibody-drug conjugate for use of embodiment 191 or embodiment 192, wherein the one or more adverse events is conjunctivitis and/or keratitis and the additional agent is a preservative-free lubricating eye drop, an ocular vasoconstrictor and/or a steroid eye drop.
197. The antibody-drug conjugate for use of any one of embodiments 101-196, wherein the antibody-drug conjugate is administered as a monotherapy.
198. The antibody-drug conjugate for use of any one of embodiments 101-197, wherein the subject is a human.
199. The antibody-drug conjugate for use of any one of embodiments 101-198, wherein the antibody-drug conjugate is in a pharmaceutical composition comprising the antibody-drug conjugate and a pharmaceutical acceptable carrier.
200. Use of an antibody-drug conjugate that binds to tissue factor (TF) for the manufacture of a medicament for treating cancer in a subject, wherein the antibody-drug conjugate comprises an anti-TF antibody or an antigen-binding fragment thereof conjugated to a monomethyl auristatin or a functional analog thereof or a functional derivative thereof, wherein the antibody-drug conjugate is administered at a dose ranging from about 0.9 mg/kg to about 2.1 mg/kg, and wherein the cancer is selected from the group consisting colorectal cancer, non-small cell lung cancer, pancreatic cancer, head and neck cancer, bladder cancer, endometrial cancer, esophageal cancer and prostate cancer.
201. The use of embodiment 200, wherein the dose is about 2.0 mg/kg.
202. The use of embodiment 200, wherein the dose is 2.0 mg/kg.
203. The use of any one of embodiments 200-202, wherein the antibody-drug conjugate is administered once about every 1 week, 2 weeks, 3 weeks or 4 weeks.
204. The use of any one of embodiments 200-203, wherein the antibody-drug conjugate is administered once about every 3 weeks.
205. The use of any one of embodiments 200-204, wherein the subject has been previously treated with one or more therapeutic agents and did not respond to the treatment, wherein the one or more therapeutic agents is not the antibody-drug conjugate.
206. The use of any one of embodiments 200-204, wherein the subject has been previously treated with one or more therapeutic agents and relapsed after the treatment, wherein the one or more therapeutic agents is not the antibody-drug conjugate.
207. The use of any one of embodiments 200-204, wherein the subject has been previously treated with one or more therapeutic agents and has experienced disease progression during treatment, wherein the one or more therapeutic agents is not the antibody-drug conjugate.
208. The use of any one of embodiments 200-207, wherein the cancer is colorectal cancer.
209. The use of embodiment 208, wherein the subject received prior systemic therapy and experienced disease progression on or after the systemic therapy.
210. The use of embodiment 209, wherein the subject received 1, 2 or 3 rounds of prior systemic therapy.
211. The use of anyone of embodiments 208-210, wherein the colorectal cancer is non-operable.
212. The use of any one of embodiments 208-211, wherein the subject has been previously treated with one or more agents selected from the group consisting of fluoropyrimidine, oxaliplatin, irinotecan and bevacizumab.
213. The use of any one of embodiments 208-212, wherein the subject has been previously treated with one or more agents selected from the group consisting of cetuximab, panitumab and a checkpoint inhibitor.
214. The use of any one of embodiments 200-207, wherein the cancer is non-small cell lung cancer.
215. The use of embodiment 214, wherein the non-small cell lung cancer is squamous cell carcinoma.
216. The use of embodiment 214 or embodiment 215, wherein the non-small cell lung cancer has predominant squamous histology.
217. The use of embodiment 216, wherein greater than 85% of the non-small cell lung cancer cells have squamous histology.
218. The use of embodiment 214, wherein the non-small cell lung cancer is adenocarcinoma.
219. The use of any one of embodiments 214-218, wherein the subject received prior systemic therapy and experienced disease progression on or after the systemic therapy.
220. The use of embodiment 219, wherein the subject received 1 or 2 rounds of prior systemic therapy.
221. The use of any one of embodiments 214-220, wherein the subject has been previously treated with one or more agents selected from the group consisting of a platinum-based therapy and a checkpoint inhibitor.
222. The use of any one of embodiments 200-207, wherein the cancer is pancreatic cancer.
223. The use of embodiment 222, wherein the pancreatic cancer is exocrine pancreatic adenocarcinoma.
224. The use of embodiment 222 or embodiment 223, wherein the pancreatic cancer has predominant adenocarcinoma histology.
225. The use of embodiment 224, wherein greater than 85% of the pancreatic cancer have adenocarcinoma histology.
226. The use of any one of embodiments 222-225, wherein the subject received prior systemic therapy and experienced disease progression on or after the systemic therapy.
227. The use of embodiment 226, wherein the subject received 1 round of prior systemic therapy.
228. The use of any one of embodiments 222-227, wherein the subject has been previously treated with one or more agents selected from the group consisting of gemcitabine and 5-fluorouracil.
229. The use of any one of embodiments 222-228, wherein the pancreatic cancer is not resectable.
230. The use of any one of embodiments 200-207, wherein the cancer is head and neck cancer.
231. The use of embodiment 230, wherein the head and neck cancer is squamous cell carcinoma.
232. The use of embodiment 230 or embodiment 231, wherein the subject received prior systemic therapy and experienced disease progression on or after the systemic therapy.
233. The use of embodiment 232, wherein, the subject received 1 or 2 rounds of prior systemic therapy.
234. The use of any one of embodiments 230-233, wherein the subject has been previously treated with one or more agents selected from the group consisting of a platinum-based therapy and a checkpoint inhibitor.
235. The use of any one of embodiments 230-234, wherein the subject has been previously treated with an anti-epithelial growth factor receptor therapy.
236. The use of any one of embodiments 200-207, wherein the cancer is bladder cancer.
237. The use of embodiment 236, wherein the subject received prior systemic therapy and experienced disease progression on or after the systemic therapy.
238. The use of embodiment 237, wherein the subject received 1, 2 or 3 rounds of prior systemic therapy.
239. The use of any one of embodiments 236-238, wherein the subject has been previously treated with a platinum-based therapy.
240. The use of any one of embodiments 236-239, wherein the subject has previously undergone surgery or radiation therapy for the bladder cancer.
241. The use of any one of embodiments 200-207, wherein the cancer is endometrial cancer.
242. The use of embodiment 241, wherein the subject received prior systemic therapy and experienced disease progression on or after the systemic therapy.
243. The use of embodiments 242, wherein the subject received 1, 2 or 3 rounds of prior systemic therapy.
244. The use of any one of embodiments 241-243, wherein the subject has been previously treated with one or more agents selected from the group consisting of a platinum-based therapy, hormone therapy, and a checkpoint inhibitor.
245. The use of any one of embodiments 241-244, wherein the subject has previously been treated with doxorubicin.
246. The use of any one of embodiments 241-245, wherein the subject has previously been treated with paclitaxel.
247. The use of any one of embodiments 241-246, wherein the subject has previously undergone surgery or radiation therapy for the endometrial cancer.
248. The use of any one of embodiments 200-207, wherein the cancer is esophageal cancer.
249. The use of embodiment 248, wherein the subject received prior systemic therapy and experienced disease progression on or after the systemic therapy.
250. The use of embodiment 249, wherein the subject received 1, 2 or 3 rounds of prior systemic therapy.
251. The use of anyone of embodiments 248-250, wherein the subject has been previously treated with one or more agents selected from the group consisting of a platinum-based therapy and a checkpoint inhibitor.
252. The use of anyone of embodiments 248-251, wherein the subject has been previously treated with one or more agents selected from the group consisting of ramucirumab, paclitaxel, 5-fluorouracil, docetaxel, irinotecan, capecitabine and trastuzumab.
253. The use of any one of embodiments 248-252, wherein the subject has previously undergone surgery, radiation therapy or endoscopic mucosal resection for the esophageal cancer.
254. The use of any one of embodiments 200-207, wherein the cancer is prostate cancer.
255. The use of embodiment 254, wherein the subject received prior systemic therapy and experienced disease progression on or after the systemic therapy.
256. The use of embodiment 255, wherein the subject received 1, 2 or 3 rounds of prior systemic therapy.
257. The use of any one of embodiments 254-256, wherein the prostate cancer is castration-resistant prostate cancer.
258. The use of any one of embodiments 254-257, wherein the subject experienced bone metastases.
259. The use of any one of embodiments 254-258, wherein the subject has been previously treated with one or more agents selected from the group consisting of androgen deprivation therapy, a luteinizing hormone-releasing hormone agonist, a luteinizing hormone-releasing hormone antagonist, a CYP17 inhibitor, and an anti-androgen.
260. The use of any one of embodiments 254-259, wherein the subject has been previously treated with one or more agents selected from the group consisting of docetaxel, prednisone and cabazitaxel.
261. The use of any one of embodiments 254-260, wherein the subject has previously undergone surgery or radiation therapy for the prostate cancer.
262. The use of any one of embodiments 200-261, wherein the cancer is an advanced stage cancer.
263. The use of embodiment 262, wherein the advanced stage cancer is a stage 3 or stage 4 cancer.
264. The use of embodiment 262 or 263, wherein the advanced stage cancer is metastatic cancer.
265. The use of any one of embodiments 200-264, wherein the cancer is recurrent cancer.
266. The use of any one of embodiments 200-265, wherein the subject received prior treatment with standard of care therapy for the cancer and failed the prior treatment.
267. The use of any one of embodiments 200-266, wherein the monomethyl auristatin is monomethyl auristatin E (MMAE).
268. The use of any one of embodiments 200-267, wherein the anti-TF antibody or antigen-binding fragment thereof of the antibody-drug conjugate is a monoclonal antibody or a monoclonal antigen-binding fragment thereof.
269. The use of any one of embodiments 200-268, wherein the anti-TF antibody or antigen-binding fragment thereof of the antibody-drug conjugate comprises a heavy chain variable region and a light chain variable region, wherein the heavy chain variable region comprises:

(i) a CDR-H1 comprising the amino acid sequence of SEQ ID NO:1;

(ii) a CDR-H2 comprising the amino acid sequence of SEQ ID NO:2; and

(iii) a CDR-H3 comprising the amino acid sequence of SEQ ID NO:3; and wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6.

270. The use of any one of embodiments 200-269, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.
271. The use of any one of embodiments 200-270, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
272. The use of any one of embodiments 200-271, wherein the anti-TF antibody of the antibody-drug conjugate is tisotumab.
273. The use of any one of embodiments 200-272, wherein the antibody-drug conjugate further comprises a linker between the anti-TF antibody or antigen-binding fragment thereof and the monomethyl auristatin.
274. The use of embodiment 273, wherein the linker is a cleavable peptide linker.
275. The use of embodiment 274, wherein the cleavable peptide linker has a formula: -MC-vc-PAB-, wherein:

### a) MC is:

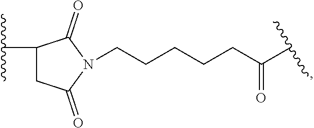 [SMILES: *C(=O)CCCCCN1C(=O)CC(*)C1=O]

b) vc is the dipeptide valine-citrulline, and

### c) PAB is:

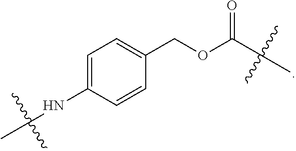 [SMILES: *Nc1ccc(COC(*)=O)cc1]

276. The use of any one of embodiments 273-275, wherein the linker is attached to sulphydryl residues of the anti-TF antibody obtained by partial reduction or full reduction of the anti-TF antibody or antigen-binding fragment thereof.
277. The use of embodiment 273, wherein the linker is attached to monomethyl auristatin E (MMAE), wherein the antibody-drug conjugate has the following structure:

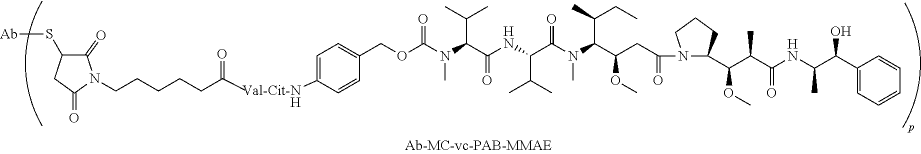

wherein p denotes a number from 1 to 8, S represents a sulphydryl residue of the anti-TF antibody, and Ab designates the anti-TF antibody or antigen-binding fragment thereof.
278. The use of embodiment 277, wherein the average value of p in a population of the antibody-drug conjugates is about 4.
279. The use of any one of embodiments 200-278, wherein the antibody-drug conjugate is tisotumab vedotin.
280. The use of any one of embodiments 200-279, wherein the route of administration for the antibody-drug conjugate is intravenous.
281. The use of anyone of embodiments 200-280, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express TF.
282. The use of any one of embodiments 200-281, wherein one or more therapeutic effects in the subject is improved after administration of the antibody-drug conjugate relative to a baseline.
283. The use of embodiment 282, wherein the one or more therapeutic effects is selected from the group consisting of: size of a tumor derived from the cancer, objective response rate, duration of response, time to response, progression free survival, overall survival and prostate specific antigen (PSA) level.
284. The use of any one of embodiments 254-261, wherein the subject exhibits a reduction in PSA level in a blood sample from the subject by at least about 5%, at least about 10%, at least about 15%, at least about 20%, at least about 25%, at least about 30%, at least about 35%, at least about 40%, at least about 45%, at least about 50%, at least about 60%, at least about 70%, or at least about 80% relative to the PSA level in a blood sample obtained from the subject before administration of the antibody-drug conjugate.
285. The use of any one of embodiments 200-284, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate.
286. The use of any one of embodiments 200-285, wherein the objective response rate is at least about 20%, at least about 25%, at least about 30%, at least about 35%, at least about 40%, at least about 45%, at least about 50%, at least about 60%, at least about 70%, or at least about 80%.
287. The use of any one of embodiments 200-286, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
288. The use of any one of embodiments 200-287, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
289. The use of any one of embodiments 200-288, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
290. The use of any one of embodiments 200-289, wherein the subject has one or more adverse events and is further administered an additional therapeutic agent to eliminate or reduce the severity of the one or more adverse events.
291. The use of any one of embodiments 200-290, wherein the subject is at risk of developing one or more adverse events and is further administered an additional therapeutic agent to prevent or reduce the severity of the one or more adverse events.
292. The use of embodiment 290 or embodiment 291, wherein the one or more adverse events is anemia, abdominal pain, hypokalemia, hyponatremia, epistaxis, fatigue, nausea, alopecia, conjunctivitis, constipation, decreased appetite, diarrhea, vomiting, peripheral neuropathy, or general physical health deterioration.
293. The use of embodiment 290 or embodiment 291, wherein the one or more adverse events is a grade 3 or greater adverse event.
294. The use of embodiment 290 or embodiment 291, wherein the one or more adverse events is a serious adverse event.
295. The use of embodiment 290 or embodiment 291, wherein the one or more adverse events is conjunctivitis and/or keratitis and the additional agent is a preservative-free lubricating eye drop, an ocular vasoconstrictor and/or a steroid eye drop.
296. The use of any one of embodiments 200-295, wherein the antibody-drug conjugate is administered as a monotherapy.
297. The use of any one of embodiments 200-296, wherein the subject is a human.
298. The use of any one of embodiments 200-297, wherein the antibody-drug conjugate is in a pharmaceutical composition comprising the antibody-drug conjugate and a pharmaceutical acceptable carrier.

### EXAMPLES

### Example 1: A Phase II Study of Tisotumab Vedotin in Subjects with Locally Advanced or Metastatic Disease in Selected Solid Tumors

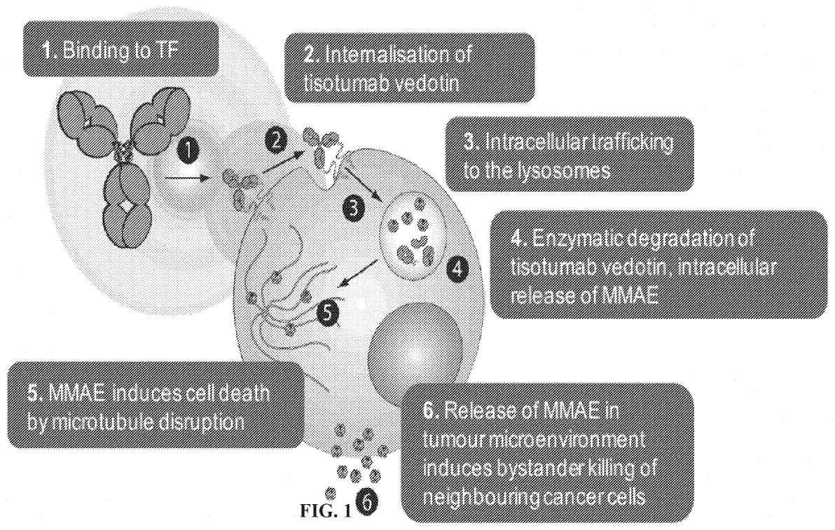

Tisotumab vedotin is an antibody-drug conjugate comprising a TF-targeted human monoclonal immunoglobulin G1 (subtype κ) conjugated via a protease-cleavable valine citrulline linker to the drug monomethyl auristatin E (MMAE), a dolastatin 10 analog. High, differential levels of TF have been observed on the membranes of neoplastic cells as well as on tumor-associated endothelium in multiple cancers, including SCCHN, NSCLC, colorectal cancer, and pancreatic cancer. Tisotumab vedotin selectively targets TF to deliver a clinically validated toxic payload to tumor cells (FIG. 1). See Breij E C et al. Cancer Res. 2014; 74(4):1214-1226 and Chu AJ. IntJInflam. 2011, 2011: Article ID 367284; doi: 10.4061/2011/367284. Dolastatins and auristatins belong to a class of chemotherapies that act as microtubule disrupting agents.

This study evaluates the efficacy, safety and tolerability of 2.0 mg/kg tisotumab vedotin in patients with inoperable, previously treated and locally advanced or metastatic colorectal cancer, non-small cell lung cancer with predominant squamous histology (squamous NSCLC), exocrine pancreatic adenocarcinoma, squamous cell carcinoma of the head and neck (SCCHN), bladder cancer, endometrial cancer, esophageal cancer or prostate cancer. Though second and third line therapeutic options are available for the patient populations in this study, response rates are low (ORRs of 15% or lower) and long term survival is poor. Patients with locally-advanced or metastatic colorectal or pancreatic cancer, squamous NSCLC, SCCHN, bladder cancer, endometrial cancer, esophageal cancer or prostate cancer whose disease has progressed after first and subsequent lines of treatment have significant unmet medical need for therapies that can meaningfully improve their prognosis.

### Methods

This global, open label, multicenter trial is designed to assess the safety, tolerability, and activity of tisotumab vedotin for the treatment of selected solid tumors. Eligible patients are at least 18 years of age with inoperable, locally advanced or metastatic cancer. Patients are enrolled into one of 8 cohorts based on tumor type, including colorectal cancer, non-small cell lung cancer with squamous cell histology only (squamous NSCLC), exocrine pancreatic adenocarcinoma, squamous cell carcinoma of the head and neck (SCCHN), bladder cancer, endometrial cancer, esophageal cancer and prostate cancer.

In all eligible patients, tisotumab vedotin is administered at a dose of 2.0 mg/kg as a 30 minutes IV infusion on Day 1 of each 21-day cycle (Q3W). For patients weighing more than 100 kg, dosing is capped at 200 mg per infusion. An individual's dose may be modified based upon treatment-related adverse events. Response is assessed every 6 weeks for the first 6 months, every 12 weeks for the next 6 months, and then every 6 months after that. RECIST v1.1 is used by the investigator to score responses for primary and secondary endpoints as well as progression. Objective responses are confirmed with repeat scans 4-6 weeks after the first documentation of response.

Inclusion criteria and exclusion criteria for patients enrolled in trial are shown in Table 1.

TABLE 1
List of inclusion and exclusion criteria

| | |
|---|---|
| Inclusion | 1. Relapsed, locally-advanced or metastatic colorectal, pancreatic cancer, |
| Criteria | squamous NSCLC, SCCHN, bladder cancer, endometrial cancer, esophageal |
| | cancer or prostate cancer that has failed prior lines of systemic treatment as |
| | specified and which are not candidates for standard therapy. |
| | Colorectal Cancer: |
| | Patients with colorectal cancer must have experienced disease |
| | progression on or after their most recent systemic therapy for non- |
| | operable metastatic disease. Isolated increase in carcinoembryonic |
| | antigen (CEA) will not qualify for study entry. Patients must have |
| | received prior therapy with each of the following agents, if eligible: a |
| | fluoropyrimidine, oxaliplatin, irinotecan, and/or bevacizumab. Patients |
| | with known/previously-tested RAS wild-type tumors and/or |
| | known/previously-tested MSI-H tumors could have received cetuximab |
| | or panitumumab and CPI, if eligible. Patients should have received no |
| | more than 3 systemic regimens in the metastatic setting. |
| | NSCLC: |
| | Patients with NSCLC must have predominant squamous histology (≥85% |
| | of cells) and must have experienced disease progression on or after their |
| | most recent systemic therapy for locally advanced or metastatic disease. |
| | Patients must have received prior therapy with a platinum-based regimen |
| | and a CPI if eligible for such therapy. Patients should have received no |
| | more than 2 systemic regimens in the locally advanced or metastatic |
| | setting. |
| | Exocrine pancreatic adenocarcinoma: |
| | Patients with exocrine pancreatic adenocarcinoma must have |
| | predominant adenocarcinoma histology (≥85% of cells) and must have |
| | experienced disease progression on or after their most recent systemic |
| | therapy for locally advanced or metastatic disease. Isolated increase in |
| | CA 19-9 or CEA will not qualify for study entry. Patients must have |
| | received prior therapy with a gemcitabine-based or 5-FU-based regimen |
| | if eligible for such therapy. Patients should have received no more than 1 |
| | systemic regimen in the unresectable or metastatic setting. |
| | SCCHN: |
| | Patients with SCCHN must have experienced disease progression on or |
| | after their most recent systemic therapy for recurrent or metastatic |
| | disease. Patients must not have tumors involving or adjacent to major |
| | blood vessels or a history of radiation involving major blood vessels in |
| | the radiation field. Patients must have received prior therapy with a |
| | platinum-based regimen and/or a CPI if eligible for such therapy. Patients |
| | eligible to receive anti-EGFR therapy must have received anti-EGFR |
| | therapy prior to study entry. Patients should have received no more than |
| | 2 systemic regimens in the recurrent/metastatic setting. |
| | Bladder cancer: |
| | Patients with bladder cancer must have experienced disease progression |
| | on or after their most recent systemic therapy for locally advanced or |
| | metastatic disease. Patients should have received no more than 3 |
| | systemic regimens in the recurrent/metastatic setting. |
| | Endometrial cancer: |
| | Patients with endometrial cancer must have experienced disease |
| | progression on or after their most recent systemic therapy for locally |
| | advanced or metastatic disease. Patients should have received no more |
| | than 3 systemic regimens in the recurrent/metastatic setting. |
| | Esophageal cancer: |
| | Patients with esophageal cancer must have experienced disease |
| | progression on or after their most recent systemic therapy for locally |
| | advanced or metastatic disease. Patients should have received no more |
| | than 3 systemic regimens in the recurrent/metastatic setting. |
| | Prostate cancer: |
| | Patients with prostate cancer must have experienced disease progression |
| | on or after their most recent systemic therapy for locally advanced or |
| | metastatic disease. Patients should have received no more than 3 |
| | systemic regimens in the recurrent/metastatic setting. |
| | 2. Measureable disease according to RECIST v1.1 as assessed by the |
| | investigator. |
| | A minimum of one non-nodal lesion ≥10 mm in the longest diameter |
| | from a non-irradiated area. If target lesion(s) are located within |
| | previously irradiated area only, the patient can be enrolled only if there |
| | has been demonstrated progression in the ″in field″lesion and upon |
| | approval of the sponsor's medical monitor. |
| | Lymph node lesion ≥15 mm in the shortest diameter from a non- |
| | irradiated area. |
| | 3. Age 18 years or older. |
| | 4. An Eastern Cooperative Oncology Group (ECOG) Performance Status |
| | score of 0 or 1. |
| | 5. The following baseline laboratory data: |
| | absolute neutrophil count (ANC) ≥1500/μL assessed at least 2 weeks |
| | after growth factor support, if applicable. |
| | platelet count ≥100 × 10⁹/L assessed at least 2 weeks after transfusion |
| | with blood products. |
| | hemoglobin ≥5.6 mmol/L (9.0 g/dL) assessed at least 2 weeks after |
| | transfusion with blood products and/or growth factor support. |
| | serum bilirubin ≤1.5 × upper limit of normal (ULN) or direct bilirubin |
| | ≤2 × ULN in patients diagnosed with Gilbert's syndrome. |
| | estimated glomerular filtration rate (eGFR) ≥60 mL/min/1.73 m² using |
| | the Modification of Diet in Renal Disease (MDRD) study equation as |
| | applicable. |
| | alanine aminotransferase (ALT) and aspartate aminotransferase (AST) |
| | ≤2.5 × ULN. (If liver tumor/metastases are present, then ≤5 × ULN is |
| | allowed). |
| | 6. Acceptable coagulation status: |
| | International normalized ratio (INR) ≤1.2 without anti-coagulation |
| | therapy. |
| | Activated partial thromboplastin time (aPTT) ≤1.25 ULN. |
| | Patients in the colorectal and pancreatic cancer cohorts who receive anti- |
| | coagulation therapy must be on a steady dose (no active titration) for at |
| | least 4 weeks prior to screening and must have an INR ≤2.5 for |
| | eligibility. Concurrent use of prophylactic acetylsalicylic acid (ASA, e.g., |
| | aspirin) for patients on anti-coagulation therapy is prohibited. Patients in |
| | the SCCHN and NSCLC who receive anti-coagulation therapy should not |
| | be enrolled. |
| | 7. Life expectancy of at least 3 months |
| | 8. Patients of childbearing potential, under the following conditions: |
| | a. Must have a negative serum or urine pregnancy test (minimum sensitivity |
| | 25 mIU/mL or equivalent units of beta human chorionic gonadotropin [β- |
| | hCG]) result within 7 days prior to the first dose of tisotumab vedotin. |
| | Patients with false positive results and documented verification that the |
| | patient is not pregnant are eligible for participation. |
| | b. Must agree not to try to become pregnant during the study and for at least |
| | 6 months after the final dose of study drug administration. |
| | c. Must agree not to breastfeed or donate ova, starting at time of informed |
| | consent and continuing through 6 months after the final dose of study |
| | drug administration |
| | d. If sexually active in a way that could lead to pregnancy, must |
| | consistently use 2 highly effective methods of birth control starting at |
| | time of informed consent and continuing throughout the study and for at |
| | least 6 months after the final dose of study drug administration. |
| | 9. Patients who can father children, under the following conditions: |
| | a. Must agree not to donate sperm starting at time of informed consent and |
| | continuing throughout the study period and for at least 6 months after the |
| | final study drug administration. |
| | b. If sexually active with a person of childbearing potential in a way that |
| | could lead to pregnancy, must consistently use 2 highly effective |
| | methods of birth control starting at time of informed consent and |
| | continuing throughout the study and for at least 6 months after the final |
| | dose of study drug administration. |
| | c. If sexually active with a person who is pregnant or breastfeeding, must |
| | consistently use one of 2 contraception options starting at time of |
| | informed consent and continuing throughout the study and for at least |
| | 6 months after the final dose of study drug administration. |
| | 10. Able to provide fresh tissue for biomarker analysis from a newly |
| | obtained core or excisional biopsy of a tumor lesion. If available, archived |
| | tumor tissue is also requested for additional biomarker analysis. |
| | 11. The patient or the patient's legally authorized representative must provide |
| | written informed consent. |
| Exclusion | 1. Patients with primary neuroendocrine or sarcomatoid histologies. |
| Criteria | 2. Hematological: Known past or current coagulation defects leading to an |
| | increased risk of bleeding; diffuse alveolar hemorrhage from vasculitis; |
| | known bleeding diathesis; ongoing major bleeding; trauma with increased |
| | risk of life-threatening bleeding or history of severe head trauma or |
| | intracranial surgery within 8 weeks of trial entry. |
| | 3. Cardiovascular: Clinically significant cardiac disease including unstable |
| | angina, acute myocardial infarction 6 months prior to screening; any medical |
| | history of congestive heart failure (Grade III or IV as classified by the New |
| | York Heart Association), any medical history of decreased cardiac ejection |
| | fraction of ≤45%. |
| | 4. Ophthalmological: Active ocular surface disease at baseline. An ocular |
| | evaluation is to be confirmed by an ophthalmologist at screening. Patients |
| | with any prior episode of cicatricial conjunctivitis or Steven Johnson |
| | syndrome (as evaluated by the investigator) are ineligible. |
| | 5. History of another malignancy within 3 years before the first dose of study |
| | drug, or any evidence of residual disease from a previously diagnosed |
| | malignancy. Exceptions are malignancies with a negligible risk of metastasis |
| | or death (e.g., 5-year overall survival ≤90%), such as adequately treated |
| | carcinoma in situ of the cervix, non-melanoma skin carcinoma, localized |
| | prostate cancer, ductal carcinoma in situ, or Stage I uterine cancer. |
| | 6. Lesions adjacent to or involving critical anatomical sites, including major |
| | blood vessels, mediastinum, and leptomeningeal disease. |
| | 7. Inflammatory bowel disease including Crohn's disease and colitis ulcerosa. |
| | 8. Ongoing, acute or chronic inflammatory skin disease. |
| | 9. Uncontrolled tumor-related pain |
| | 10. Inflammatory lung disease, including moderate and severe asthma and |
| | chronic obstructive pulmonary disease, requiring chronic medical therapy |
| | 11. Medications or treatment regimens: |
| | For patients with SCCHN or NSCLC, therapeutic anti-coagulation is not |
| | permitted. For patients with colorectal or pancreatic cancers, therapeutic |
| | anti-coagulation therapy is permitted IF the patient is no longer being |
| | actively titrated for anti-coagulation. For oral anticoagulation therapy, |
| | colorectal and pancreatic patients must be on steady doses for at least 4 |
| | weeks prior to the first dose of study drug. |
| | Chronic prophylactic treatment with ASA (e.g., aspirin) in combination |
| | with other anti-coagulation therapy is prohibited. |
| | Cumulative dose of corticosteroid ≥150 mg (prednisone or equivalent |
| | doses of corticosteroids) within 2 weeks of the first tisotumab vedotin |
| | administration is prohibited. |
| | 12. Surgery/procedures: Major surgical procedure |
| | (defined as a surgery requiring inpatient hospitalization |
| | of at least 48 hours) within 4 weeks or excisional |
| | biopsy within 7 days prior to the first study drug administration. Patients who |
| | have planned major surgery during the treatment period must be excluded |
| | from the trial. |
| | 13. Received a live vaccine within 30 days prior to the first dose of trial |
| | treatment. Examples of live vaccines include, but are not limited to, the |
| | following: measles, mumps, rubella, varicella/zoster (chicken pox), yellow |
| | fever, rabies, Bacillus Calmette—Guérin, and typhoid vaccine. Seasonal |
| | influenza vaccines for injection are generally killed virus vaccines and are |
| | allowed; however, intranasal influenza vaccines (e.g., FLUIMIST ^{®}) are live |
| | attenuated vaccines and are not allowed. |
| | 14. Peripheral neuropathy Grade ≥2. |
| | 15. Patients with clinical symptoms or signs of gastrointestinal obstruction |
| | and who require parental hydration and/or nutrition. |
| | 16. Prior therapy: |
| | Any prior treatment with MMAE-derived drugs. |
| | Radiotherapy within 21 days prior to the first administration of study |
| | drug. Patients must have recovered from all radiation-related toxicities. |
| | At least 42 days must have elapsed from the last administration of |
| | chemo-radiotherapy. |
| | Small molecules, chemotherapy, immunotherapy, biologics, experimental |
| | agents, or any other antitumor therapy within 21 days prior to the first |
| | administration of study drug. If underlying disease is progressing on |
| | treatment, patients may enroll within 21 days upon approval of the |
| | sponsor's medical monitor. Patients must have recovered from all related |
| | toxicities. |
| | 17. Any uncontrolled Grade 3 or higher (per the NCI CTCAE v5.0) viral, |
| | bacterial, or fungal infection within 2 weeks prior to the first dose of |
| | tisotumab vedotin. Routine antimicrobial prophylaxis is permitted. |
| | 18. Known seropositivity of human immunodeficiency virus; known medical |
| | history of hepatitis B or C infection. |
| | 19. Known history of brain metastasis or active brain metastasis. |
| | Patients with symptoms of brain metastasis should be screened for this |
| | condition prior to enrollment. |
| | 20. Patients who are breastfeeding, pregnant, or planning to become pregnant |
| | from time of informed consent until 6 months after final dose of study drug |
| | administration. |
| | 21. Known hypersensitivity to any excipient contained in the drug |
| | formulation of tisotumab vedotin. |
| | 22. Grade 3 or higher pulmonary disease unrelated to underlying malignancy. |
| | 23. Other serious underlying medical condition that, in the opinion of the |
| | investigator, would impair the patient's ability to receive or tolerate the |
| | planned treatment and follow-up. |

Lyophilized vials containing 40 mg of tisotumab vedotin are stored in a refrigerator at 2° C. to 8° C. Tisotumab vedotin is reconstituted in 4 ml of water leading to a reconstituted solution comprising 10 mg/mL tisotumab vedotin. The reconstituted tisotumab vedotin is diluted into a 0.9% NaCl 100 mL infusion bag according to the dose calculated for the patient to receive 2.0 mg/kg tisotumab vedotin. Intravenous infusion is completed within 24 hours after the tisotumab vedotin vial has been reconstituted. A 0.2 μm in-line filter is used for the intravenous infusion. The entire 100 mL volume from the prepared infusion bag is administered. No dead volume is provided. For patients that do not tolerate the protocol-specified dosing schedule, dose reductions are permitted in order to allow the patient to continue treatment with tisotumab vedotin (Table 2).

TABLE 2
Dose Modification Scheme

| Previous dose of | Reduced dose of |
|---|---|
| tisotumab vedotin | tisotumab vedotin |
| 2.0 mg/kg (200 mg maximum total dose) | 1.3 mg/kg |
| | (130 mg maximum dose) |
| 1.3 mg/kg (130 mg maximum dose) | 0.9 mg/kg |
| | (90 mg maximum dose) |
| 0.9 mg/kg (90 mg maximum dose) | 0.9 mg/kg* |
| | (90 mg maximum dose) |

*If the patient is already being treated with tisotumab vedotin 0.9 mg/kg, the dose of tisotumab vedotin is not reduced further.

Objectives and endpoints are described in Table 3. The confirmed objective response rate (ORR) is defined as the proportion of patients who achieve a confirmed CR or PR according to RECIST v1.1 as assessed by the investigator. The confirmed ORR of each cohort and its exact 2-sided 95% CI using the Clopper-Pearson method is calculated.

Confirmed and unconfirmed ORR is defined as the proportion of patients who achieve a CR or PR according to RECIST v1.1 as assessed by the investigator. These include patients with confirmed responses as well as those whose responses were not confirmed or had not yet been assessed for confirmation. DCR is defined as the proportion of patients who achieve a CR or PR according to RECIST v1.1 as assessed by the investigator, or meet the SD criteria at least once after start of study treatment at a minimum interval of 12 weeks. The confirmed and unconfirmed ORR and the DCR are estimated for each cohort and the 95% CIs are calculated using the Clopper-Pearson method.

DOR is defined as the time from the first documentation of objective response (CR or PR that is subsequently confirmed) to the first documentation of PD or death due to any cause, whichever comes first. TTR is defined as the time from the start of study treatment to the first documentation of objective response (CR or PR that is subsequently confirmed). PFS is defined as the time from the start of study treatment to the first documentation of PD or death due to any cause, whichever comes first. OS is defined as the time from the start of study treatment to date of death due to any cause. In the absence of death, survival time will be censored at the last date the patient is known to be alive (i.e., date of last contact). The DOR, TTR, PFS, and OS are estimated for each cohort using the Kaplan-Meier methodology, and the medians and associated 95% CIs are calculated. Kaplan-Meier plots are provided as appropriate. The 3- and 6-month PFS rates, as well as the 6- and 12-month OS rates, are summarized. In addition, the TTR of patients who achieve an objective response is summarized.

TABLE 3
Objectives and endpoints

| Primary Objective | Corresponding Primary Endpoint |
|---|---|
| Evaluate antitumor activity of tisotumab | Investigator-determined confirmed ORR as |
| vedotin | measured by RECIST v1.1 |
| Secondary Objectives | Corresponding Secondary Endpoints |
| Evaluate the safety and tolerability of | Type, incidence, severity, seriousness, and |
| tisotumab vedotin | relatedness of AEs |
| Evaluate preliminary antitumor activity of | Investigator-determined confirmed and |
| tisotumab vedotin | unconfirmed ORR as measured by RECIST |
| | v 1.1 |
| Evaluate stability and control of disease | Investigator-determined disease control rate |
| | (DCR) as measured by RECIST v1.1 |
| Evaluate durability of response in patients | Investigator-determined duration of |
| who respond to tisotumab vedotin | response (DOR) as measured by RECIST |
| | v1.1 |
| Evaluate the timing of responses | Investigator-determined time to response |
| | (TTR) as measured by RECIST v1.1 |
| Evaluate progression-free survival (PFS) of | Investigator-determined PFS as measured |
| patients treated with tisotumab vedotin | by RECIST v1.1 |
| Evaluate survival of patients treated with | Overall survival (OS) |
| tisotumab vedotin | |
| Assess pharmacokinetics of tisotumab | Selected PK parameters for tisotumab |
| vedotin | vedotin and MMAE |
| Assess immunogenicity of tisotumab vedotin | Incidence of anti-therapeutic antibodies |
| | (ATAs) to tisotumab vedotin |
| Additional Objectives | Corresponding Additional Endpoints |
| Evaluate Tissue Factor expression-response | TF expression-response relationship |
| relationship | following treatment with tisotumab vedotin |
| Assess biomarkers of biological activity and | Relationship between biomarkers in blood |
| resistance and predictive biomarkers of | and tumor tissue to efficacy, safety, or other |
| response | biomarker endpoints following treatment |
| | with tisotumab vedotin |

Patients continue to receive tisotumab vedotin treatment until disease progression, unacceptable toxicity, investigator decision, consent withdrawal, start of a subsequent anticancer therapy, study termination by the sponsor, pregnancy, or death, whichever comes first. Patients are followed for response assessments until disease progression, subsequent cancer therapy, study termination by the sponsor, or death, whichever comes first. After treatment discontinuation, all patients are followed for subsequent cancer therapies and survival.

Adverse events of special interest include ocular adverse events, infusion-related reactions, increased bleeding, hemorrhage, elevated liver enzymes, mucositis, neutropenia, and peripheral neuropathy. In order to prevent ocular AEs, the following ocular pre-medication guidelines are followed: (1) Administration of local ocular vasoconstrictor before infusion (brimonidine tartrate 0.2% eye drops or similar, 3 drops in each eye immediately prior to start of infusion; otherwise to be used in accordance with the product prescribing information). If the patient does not tolerate ocular vasoconstrictors due to adverse reactions, continued treatment with these may be stopped at the discretion of the investigator and following discussion with the sponsor's medical monitor. (2) Use of refrigerator-based eye cooling pads during infusion, e.g., THERA PEARL® Eye Mask or similar. To be applied immediately before infusion in accordance with the instructions provided with the eye cooling pads. (3) Application of steroid eye drops (dexamethasone 0.1% eye drops or equivalent) during the first 3 days of each treatment cycle (i.e., first drop to be given prior to start of infusion; continue treatment for 72 hours thereafter). Steroid eye drops should be administered as 1 drop in each eye, 3 times daily, for 3 days, or used in accordance with the product prescribing information. (4) Use of preservative-free lubricating eye drops during the whole treatment phase of the trial (i.e., from first dose of study drug until 30 days after the last dose of study drug). Lubricating eye drops should be administered according to the product prescribing information. (5) It is recommended not to wear contact lenses while being treated with tisotumab vedotin from the first dose until 30 days after the last dose of study drug.

Tisotumab vedotin may cause Infusion-Related Reactions including severe hypersensitivity or anaphylaxis. Signs and symptoms usually develop during or shortly after drug infusion. In case any clinical significant IRR is observed during or after the first infusion of tisotumab vedotin or at subsequent treatment cycles, the patient should be observed for 2 hours after the end of tisotumab vedotin administration for all subsequent infusions. At all times during infusion, immediate emergency treatment of an anaphylactic reaction according to institutional standards must be assured. In order to treat possible anaphylactic reactions, for instance, dexamethasone 10 mg and epinephrine in a 1:1000 dilution or equivalents must always be available along with equipment for assisted ventilation.

### Example 2: Anti-Tumor Activity of Tisotumab Vedotin in Cell Line-Derived and Patient-Derived Xenograft Mouse Models of Non-Small-Cell Lung Carcinoma

The in vivo anti-tumor efficacy of tisotumab vedotin was tested in xenograft mouse models for non-small-cell lung carcinoma (NSCLC), either of the squamous cell carcinoma (SCC) or the adenocarcinoma (AC) subtype.

A NCI-H441 (papillary adenocarcinoma of the lung, ATCC cat. No. HTB-174) cell line-derived xenograft (CDX) model was induced by subcutaneous injection in the flank of female immunodeficient SCID mice with 200 μL tumor cell suspension containing five million cells on day 0. Tumor volumes were measured at least twice per week using a digital caliper. Tumor volumes (mm³) were calculated as follows: tumor volume=0.52×(length)×(width)². Mice were treated by intraperitoneal injection of tisotumab vedotin at various doses (0.5, 1.5, or 4.5 mg/kg) once on day 27 to evaluate dose-dependent anti-tumor effect of tisotumab vedotin. In the control groups, mice were treated with isotype control antibody IgG1-b12 at 4 mg/kg, or with isotype ADC control IgG1-b12-vcMMAE at 0.5, 1.5, or 4.5 mg/kg.

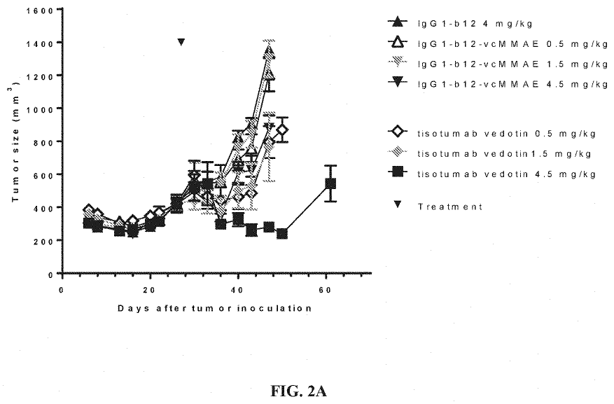

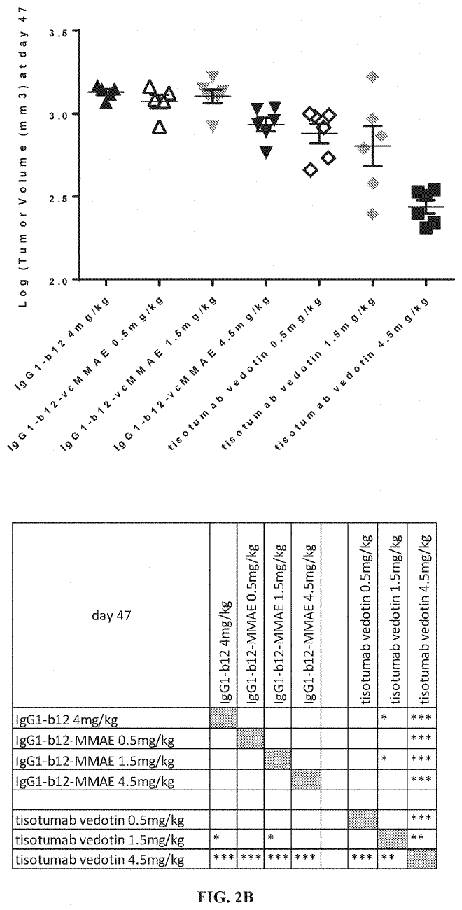

As shown in FIG. 2A, treatment with tisotumab vedotin at 4.5 mg/kg showed superior efficacy in comparison with the other treatment groups in the NCI-H441 CDX model. Treatment with tisotumab vedotin at 1.5 mg/kg and particularly at 4.5 mg/kg significantly inhibited tumor development on day 47 compared to treatment with IgG1-b12-vcMMAE at corresponding doses (FIG. 2B).

Patient-derived xenograft (PDX) mouse models of NSCLC were also produced. Patient-derived tumor fragments were removed from donor mice and cut into 4-5 mm fragments. Fragments were implanted subcutaneously in the flank of nude mice, under isofluorane anesthesia, to allow tumor growth. At a tumor volume of 80-200 mm³ (i.e., day 0), mice were randomized into different groups. Mice received intravenous administrations of tisotumab vedotin, IgG1-b12 control, or IgG1-b12-vcMMAE control at 4 mg/kg on days 0 and 7 respectively. Tumor growth was calculated by measuring the tumor volumes every 3-4 days. Efficacy of tisotumab vedotin was assessed in NSCLC models LXFE 690 (subtype SCC), LXFE 772 (subtype SCC), LXFA 289 (subtype AC), LXFA 1041 (subtype AC), LXFA 1674 (subtype AC) and LUO 395 (subtype SCC) respectively.

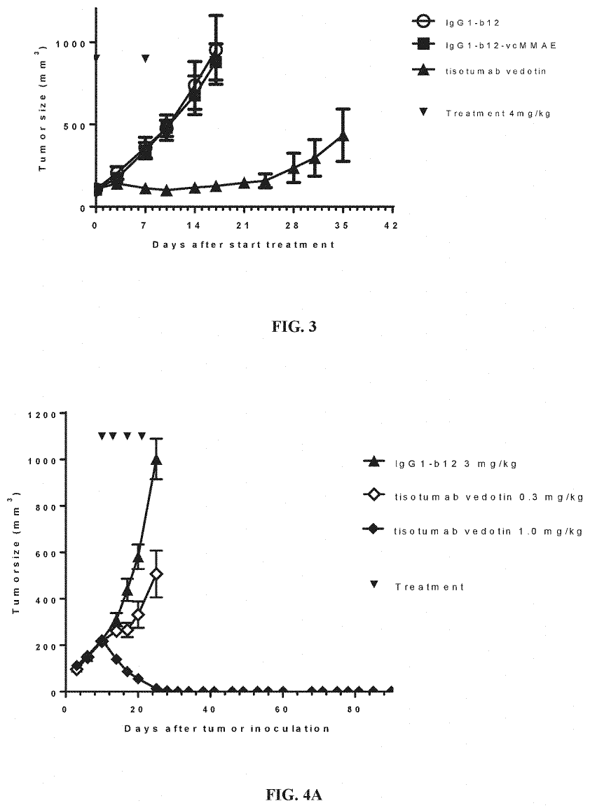

FIG. 3 shows exemplary efficacy results of tisotumab vedotin in a squamous cell lung carcinoma model LXFE 690. In this model, a strong and significant anti-tumor effect with two doses of 4 mg/kg of tisotumab vedotin was observed. Tisotumab vedotin also showed anti-tumor activity in the LXFE 772, LXFA 289, LXFA 1041, LXFA 1674 and LUO 395 NSCLC xenograft models.

### Example 3: Anti-Tumor Activity of Tisotumab Vedotin in Cell Line-Derived and Patient-Derived Xenograft Mouse Models of Pancreatic Cancer

The in vivo anti-tumor efficacy of tisotumab vedotin was tested in xenograft mouse models for pancreatic cancer.

A CDX model using HPAF-II cells (pancreatic adenocarcinoma, ATCC, cat. No. CRL-1997) was induced by injecting subcutaneously in the flank of SCID mice with 200 μL tumor cell suspension containing 2×10⁶ cells on day 0. On days 10, 13, 17 and 20, the mice received intraperitoneal administration of tisotumab vedotin at a dose of 0.3 mg/kg or 1 mg/kg or IgG1-b12 control at 3 mg/kg.

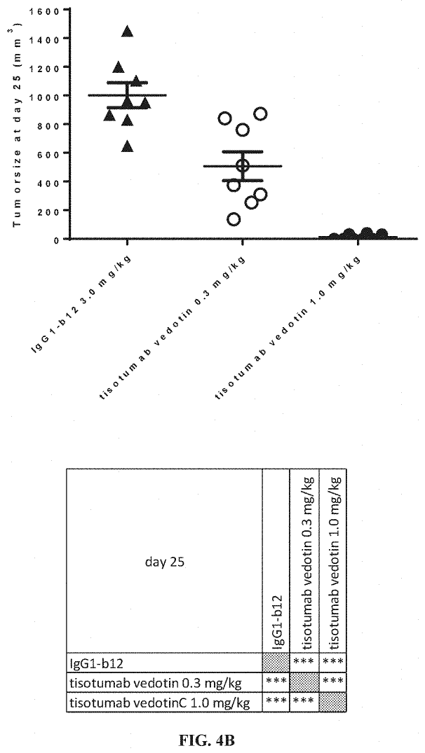

As shown in FIG. 4, in the HPAF-II CDX model, treatment with tisotumab vedotin at 0.3 mg/kg resulted in a partial response compared to the IgG-b12 treated controls. Treatment with tisotumab vedotin at 1.0 mg/kg resulted in complete tumor regression.

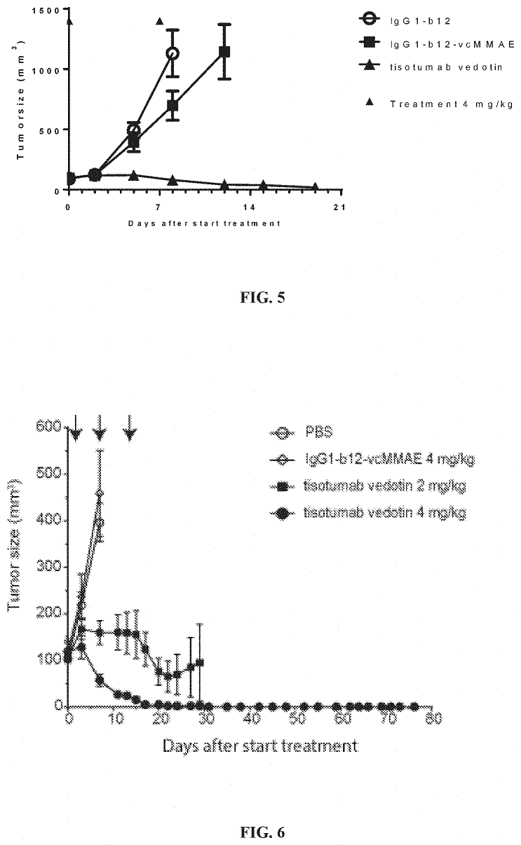

PDX models for pancreatic cancer were also produced, and anti-tumor efficacy of tisotumab vedotin was demonstrated in the PAXF 1657 and PA5415 PDX models. In each model, at a tumor volume of 80-200 mm³ (this was labeled as day 0 in the experiment), mice were randomized into different groups. Mice received intravenous administrations of tisotumab vedotin, IgG1-b12 control, or IgG1-b12-vcMMAE control at 4 mg/kg on days 0 and 7 respectively. FIG. 5 shows exemplary efficacy results of tisotumab vedotin in the PAXF 1657 model.

### Example 4: Anti-Tumor Activity of Tisotumab Vedotin in Cell Line-Derived Xenograft Mouse Models of Head and Neck Cancer

The in vivo anti-tumor efficacy of tisotumab vedotin was tested in xenograft mouse models for head and neck cancer.

Squamous cell carcinoma of the head and neck (SCCHN) cell lines FaDu (ATCC cat. No. HTB-43), VU-SCC-040 and VU-SCC-OE (Hermsen et al (1996). Genes Chromosomes. Cancer 15:1-9) were used to produce CDX mice models of SCCHN. FaDu and VU-SCC-040 cell lines and xenograft tumors both had abundant TF expression. In comparison, the VU—SCC-OE cell line and xenograft tumors had significantly less, but detectable levels of TF expression.

Cells from these SCCHN cell lines were subcutaneously injected in both flanks of nude mice at approximately 2×10⁶ cells/per flank. When tumors reached an average size of 100 mm³ (range 40-180 mm³; day 0), intraperitoneal treatment of the mice with tisotumab vedotin was started. Mice received three weekly treatment (i.e., on days 0, 7 and 14) with tisotumab vedotin at 2 mg/kg or 4 mg/kg, or control treatment with phosphate buffer saline (PBS) or IgG1-b12-vcMMAE at 4 mg/kg. Mice were sacrificed when tumor volume reached 5 times the start tumor volume in one of the two flanks and/or displayed tumor ulceration, body weight loss ≥20% or moribund appearance. Tumor volume was measured using electronic calipers (V=(L×W×H)×0.5 where V=volume, L=length, W=width, H=height), and calculated as mean volume of tumor(s) per mouse. Tumors with a start volume below 40 mm³ were excluded from the analysis.

Tisotumab vedotin had anti-tumor effects in all three SCCHN CDX models, ranging from inhibition of tumor growth to complete tumor regression. FIG. 6 shows the effect of tisotumab vedotin treatment in the FaDu CDX model. In the PBS or IgG1-b12-vcMMAE treated mice from the control groups, tumor outgrowth was rapid and the majority of mice had to be sacrificed on day 7. In mice treated with tisotumab vedotin at 2 mg/kg, tumor growth was significantly inhibited, and tumor regression was observed after 3 doses. However, tumors started to re-grow by day 30. In mice treated with tisotumab vedotin at 4 mg/kg, significant tumor regression was observed after the first dose. Moreover, complete tumor regression was observed by day 30 in all mice without recurrence of tumors until the end of the experiment (i.e., day 76).

### Example 5: Anti-Tumor Activity of Tisotumab Vedotin in a Bladder Cancer Patient-Derived Xenograft Model

The in vivo anti-tumor efficacy of tisotumab vedotin was tested in the BXF1036 patient-derived xenograft mouse model for bladder cancer. The model was performed at Oncotest GmbH (Germany).

Tumor fragments were removed from donor mice, cut into 4-5 mm fragments and implanted subcutaneously in the flank of athymic nude (NMRI nu/nu) mice, under isofluorane anesthesia. At a tumor volume of 50-250 mm³, mice were randomized and treated intravenously with a single dose of 0.5, 1, 2 or 4 mg/kg tisotumab vedotin, the isotype control ADC IgG1-b12-MMAE (4 mg/kg) or the unconjugated isotype control antibody IgG1-b12 (4 mg/kg) diluted in PBS. The day of randomization and treatment was designated day 0. Tumor growth was assessed every 3-4 days by two-dimensional measurement with a caliper. Tumor volumes were calculated according to the following formula: tumor volume (mm³)=0.5*(a*b²), in which “a” represents the largest and “b” the perpendicular tumor diameter.

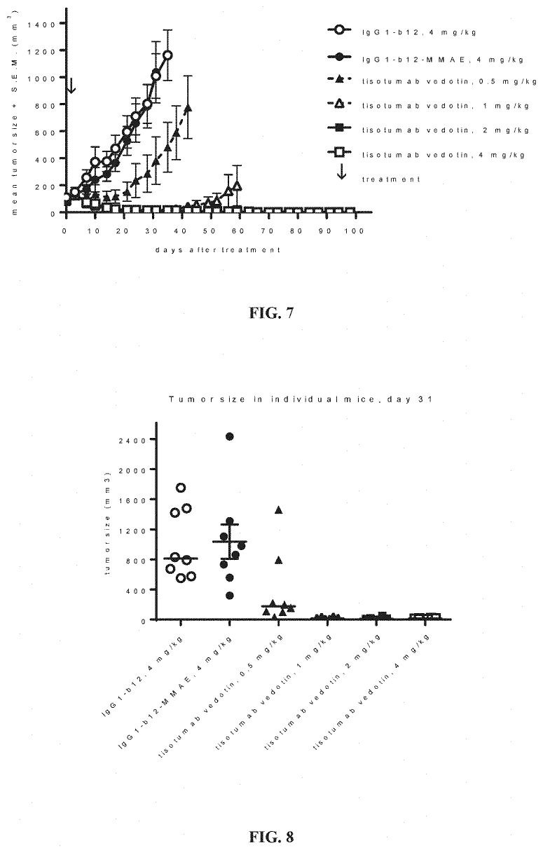

Tisotumab vedotin induced anti-tumor activity in the BXF 1036 bladder cancer xenograft model at all treatment doses, whereas the isotype control ADC (IgG1-b12-MMAE) did not inhibit tumor growth (FIG. 7 and FIG. 8).

### Example 6: Anti-Tumor Activity of Tisotumab Vedotin in an Esophageal Cancer PDX Model

The in vivo anti-tumor efficacy of tisotumab vedotin was tested in an esophageal cancer PDX model (ES0195), derived from a human esophageal cancer tumor specimen. The study was performed at Crown Bio (China).

Tumor fragments were removed from donor mice, cut into fragments (2-3 mm in diameter) and implanted subcutaneously in the flank of BalB/c nude mice. At an average tumor volume of 143 mm³, mice were randomized into treatment groups according to their tumor sizes (8 mice per group). On the same day, animals were treated intravenously with 4 mg/kg tisotumab vedotin, the isotype control ADC IgG1-b12-MMAE or the unconjugated isotype control antibody IgG1-b12 diluted in PBS. The day of randomization and first treatment was designated day 0. A second treatment was administered at day 7.

Tumor growth was assessed every 3-4 days by two-dimensional measurement with a caliper. Tumor volumes were calculated according to the following formula: tumor volume (mm³)=0.5*(a*b²), in which “a” represents the largest and “b” the perpendicular tumor diameter.

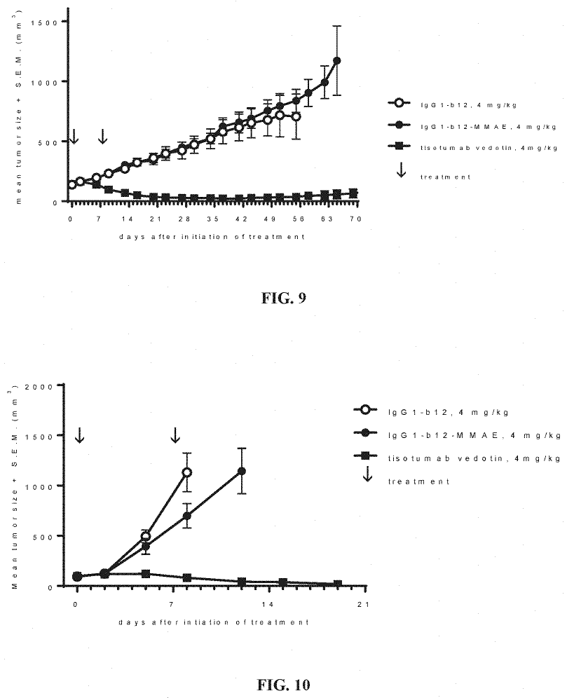

Tisotumab vedotin induced potent anti-tumor activity in the ES0195 esophageal cancer xenograft model whereas the isotype control ADC (IgG1-b12-MMAE) did not inhibit tumor growth (FIG. 9).

### Example 7: Anti-Tumor Activity of Tisotumab Vedotin in a Pancreatic Cancer Patient-Derived Xenograft Model

The in vivo anti-tumor efficacy of tisotumab vedotin was tested in two different pancreatic cancer patient-derived xenograft models, originally derived from human pancreatic cancer tumor specimens.

The study using the PAXF 1657 pancreatic cancer patient-derived xenograft model was performed at Oncotest GmbH (Germany). Tumor fragments were removed from donor mice, cut into 4-5 mm fragments and implanted subcutaneously in the flank of athymic nude (NMRI nu/nu) mice, under isofluorane anesthesia. At a tumor volume of 100-200 mm³, mice were randomized into groups of 8 mice with an equal tumor size distribution, and treated intravenously with 4 mg/kg tisotumab vedotin, the isotype control ADC IgG1-b12-MMAE or the unconjugated isotype control antibody IgG1-b12 diluted in PBS. The day of randomization and first treatment was designated day 0. A second treatment was administered at day 7. Tumor growth was assessed every 3-4 days by two-dimensional measurement with a caliper. Tumor volumes were calculated according to the following formula: tumor volume (mm³)=0.5*(a*b²), in which “a” represents the largest and “b” the perpendicular tumor diameter.

Tisotumab vedotin induced potent anti-tumor activity in the PAXF 1657 pancreatic cancer xenograft model (FIG. 10).

The study using the PA5415 pancreatic cancer patient-derived xenograft model was performed at Crown Bio (San Diego, U.S.). Patient-derived tumor cell suspensions (PA5415) were thawed, washed PBS and resuspended in cold PBS at concentrations of 74,000 viable cells/100 μl. Cell suspensions were mixed with an equal volume of Cultrex® extracellular matrix (ECM) and kept on ice. Female non-obese diabetic severe combined immunodeficient (NOD-SCID) mice were injected subcutaneously with 200 μl of the cell suspension in ECM, under isoflurane anesthesia (day−37). Tumor volumes were calculated according to the following formula: tumor volume (mm³)=0.5*(a*b²), in which “a” represents the largest and “b” the shortest tumor diameter. At an average tumor size of 215 mm³, mice were randomized into groups of 8 mice with comparable tumor size distribution. On the same day, mice were treated intravenously with tisotumab vedotin (0.5, 1 or 2 mg/kg), the isotype control ADC IgG1-b12-MMAE (2 mg/kg) or the unconjugated isotype control antibody IgG1-b12 (2 mg/kg) diluted in PBS. The day of randomization and first treatment was designated day 0. A second treatment was administered at day 7. Tumor growth was assessed every 3-4 days.

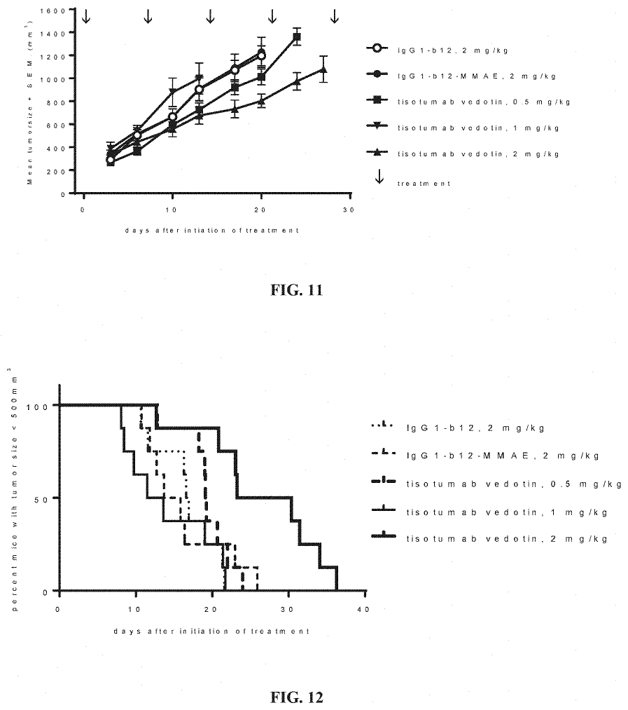

At a dose of 2 mg/kg, tisotumab vedotin induced inhibition of tumor growth in the PA5415 pancreatic cancer xenograft model (FIG. 11). Moreover, tisotumab vedotin prolonged tumor-free survival (using a tumor size of 500 mm³ as a cut-off for tumor progression; FIG. 12).

### Example 8: Anti-Tumor Activity of Tisotumab Vedotin in a Colorectal Cancer PDX Mouse Model

The potential of tisotumab vedotin for the treatment of colorectal cancer was evaluated herein.

The in vivo anti-tumor efficacy of tisotumab vedotin was evaluated in a diverse panel of colorectal cancer (CRC) patient-derived xenograft (PDX) models in NOD-SCID mice in a “mouse clinical trial” (MCT). In this MCT, a large set of PDX models (n=33) was screened for TV sensitivity using one mouse per treatment group. Xenografts were derived from frozen tumor cells from cancer patients. Establishment and characterization of the PDX models was performed following subcutaneous injection of 100 of the PDX tumor cell suspension into the rear flank of the mouse. Tumor size was determined by caliper measurement two times a week and tumor volume was calculated as 0.5×length×width². When tumors reached the volume of 150-250 mm³, mice were randomized into 2 groups per PDX model: either the tisotumab vedotin-treated group or the PBS control group (one mouse in each arm, n=1). Mice were administered the following treatments by intravenous injections: 1) tisotumab vedotin alone at dose level 2 mg/kg (dose volume 10 ml/kg) weekly for two weeks (QW×2); 2) PBS control (dose volume 10 ml/kg) weekly for two weeks (QW×2).

Evaluation of response to treatment with tisotumab vedotin was performed by comparing the change in tumor volume of mice treated with tisotumab vedotin (ΔT=tumor volume last day of analysis treated mouse—tumor volume day 0 treated mouse) with the change in tumor volume of PBS-treated control mice (ΔC=tumor volume last day of analysis control mouse—tumor volume day 0 control mouse). The relative tumor growth was defined as follows:

Relative tumor growth=ΔT/ΔC*100

Response was evaluated between day 7 and day 25, when exposure could reasonably be assumed. Models were excluded from the final analysis if the control tumor did not show at least doubling in tumor volume compared to day 0. Responding models (R) were defined as models showing ΔT/ΔC<10% (tumor stasis or tumor regression), and non-responding models were defined as ΔT/ΔC>70%. The models that could not be classified as responder or non-responder (10%<ΔT/ΔC<70%), were classified as intermediate.

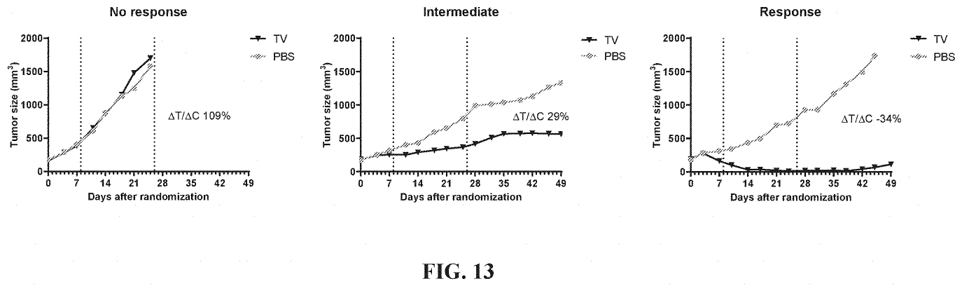

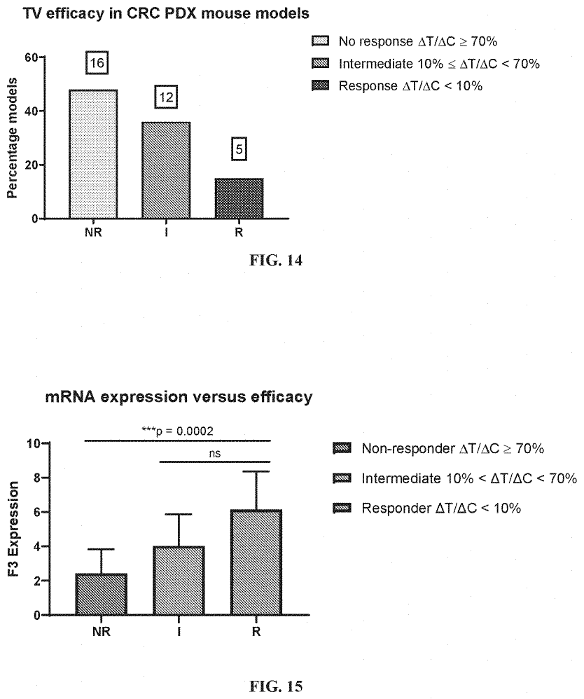

As shown in FIG. 13 and FIG. 14, tisotumab vedotin induced potent anti-tumor activity (tumor stasis or tumor regression) in 5/33 of the PDX models and no response in 16/33 of the models. 12/33 of the PDX models were classified as intermediate. FIG. 15 demonstrates average TF mRNA expression levels in PDX models classified as responder, non-responder or intermediate. There was a significant difference in the amount of TF mRNA observed in the PDX models in the responder group compared to the PDX models of the non-responder group (p=0.0002). No difference in TF mRNA expression was observed between the PDX models of the responder group and the PDX models of the intermediate group (p=0.0654).

What is claimed is:

1. A method of treating cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof, wherein the antibody-drug conjugate is administered at a dose ranging from about 1.5 mg/kg to about 2.1 mg/kg, and wherein the cancer is selected from the group consisting of colorectal cancer, non-small cell lung cancer, pancreatic cancer, head and neck cancer, bladder cancer, endometrial cancer, esophageal cancer and prostate cancer.

2. The method of claim 1, wherein the dose is about 2.0 mg/kg.

3. The method of claim 1, wherein the dose is 2.0 mg/kg.

4. The method of any one of claims 1-3, wherein the antibody-drug conjugate is administered once about every 1 week, 2 weeks, 3 weeks or 4 weeks.

5. The method of any one of claims 1-4, wherein the antibody-drug conjugate is administered once about every 3 weeks.

6. The method of any one of claims 1-5, wherein the subject has been previously treated with one or more therapeutic agents and did not respond to the treatment, wherein the one or more therapeutic agents is not the antibody-drug conjugate.

7. The method of any one of claims 1-5, wherein the subject has been previously treated with one or more therapeutic agents and relapsed after the treatment, wherein the one or more therapeutic agents is not the antibody-drug conjugate.

8. The method of any one of claims 1-5, wherein the subject has been previously treated with one or more therapeutic agents and has experienced disease progression during treatment, wherein the one or more therapeutic agents is not the antibody-drug conjugate.

9. The method of any one of claims 1-8, wherein the cancer is colorectal cancer.

10. The method of claim 9, wherein the subject received prior systemic therapy and experienced disease progression on or after the systemic therapy.

11. The method of claim 10, wherein the subject received 1, 2 or 3 rounds of prior systemic therapy.

12. The method of any one of claims 9-11, wherein the colorectal cancer is non-operable.

13. The method of any one of claims 9-12, wherein the subject has been previously treated with one or more agents selected from the group consisting of fluoropyrimidine, oxaliplatin, irinotecan and bevacizumab.

14. The method of any one of claims 9-13, wherein the subject has been previously treated with one or more agents selected from the group consisting of cetuximab, panitumab and a checkpoint inhibitor.

15. The method of any one of claims 1-8, wherein the cancer is non-small cell lung cancer.

16. The method of claim 15, wherein the non-small cell lung cancer is squamous cell carcinoma.

17. The method of claim 15 or claim 16, wherein the non-small cell lung cancer has predominant squamous histology.

18. The method of claim 17, wherein greater than 85% of the non-small cell lung cancer cells have squamous histology.

19. The method of embodiment 15, wherein the non-small cell lung cancer is adenocarcinoma.

20. The method of any one of claims 15-19, wherein the subject received prior systemic therapy and experienced disease progression on or after the systemic therapy.

21. The method of claim 20, wherein the subject received 1 or 2 rounds of prior systemic therapy.

22. The method of any one of claims 15-21, wherein the subject has been previously treated with one or more agents selected from the group consisting of a platinum-based therapy and a checkpoint inhibitor.

23. The method of any one of claims 1-8, wherein the cancer is pancreatic cancer.

24. The method of claim 23, wherein the pancreatic cancer is exocrine pancreatic adenocarcinoma.

25. The method of claim 23 or claim 24, wherein the pancreatic cancer has predominant adenocarcinoma histology.

26. The method of claim 25, wherein greater than 85% of the pancreatic cancer cells have adenocarcinoma histology.

27. The method of any one of claims 23-26, wherein the subject received prior systemic therapy and experienced disease progression on or after the systemic therapy.

28. The method of claim 27, wherein the subject received 1 round of prior systemic therapy.

29. The method of any one of claims 23-28, wherein the subject has been previously treated with one or more agents selected from the group consisting of gemcitabine and 5-fluorouracil.

30. The method of any one of claims 23-29, wherein the pancreatic cancer is not resectable.

31. The method of any one of claims 1-8, wherein the cancer is head and neck cancer.

32. The method of claim 31, wherein the head and neck cancer is squamous cell carcinoma.

33. The method of claim 31 or claim 32, wherein the subject received prior systemic therapy and experienced disease progression on or after the systemic therapy.

34. The method of claim 33, wherein, the subject received 1 or 2 rounds of prior systemic therapy.

35. The method of any one of claims 31-34, wherein the subject has been previously treated with one or more agents selected from the group consisting of a platinum-based therapy and a checkpoint inhibitor.

36. The method of any one of claims 31-35, wherein the subject has been previously treated with an anti-epithelial growth factor receptor therapy.

37. The method of any one of claims 1-8, wherein the cancer is bladder cancer.

38. The method of claim 37, wherein the subject received prior systemic therapy and experienced disease progression on or after the systemic therapy.

39. The method of claim 38, wherein the subject received 1, 2 or 3 rounds of prior systemic therapy.

40. The method of any one of claims 37-39, wherein the subject has been previously treated with a platinum-based therapy.

41. The method of any one of claims 37-40, wherein the subject has previously undergone surgery or radiation therapy for the bladder cancer.

42. The method of any one of claims 1-8, wherein the cancer is endometrial cancer.

43. The method of claim 42, wherein the subject received prior systemic therapy and experienced disease progression on or after the systemic therapy.

44. The method of claim 43, wherein the subject received 1, 2 or 3 rounds of prior systemic therapy.

45. The method of any one of claims 42-44, wherein the subject has been previously treated with one or more agents selected from the group consisting of a platinum-based therapy, hormone therapy, and a checkpoint inhibitor.

46. The method of any one of claims 42-45, wherein the subject has previously been treated with doxorubicin.

47. The method of any one of claims 42-46, wherein the subject has previously been treated with paclitaxel.

48. The method of any one of claims 42-47, wherein the subject has previously undergone surgery or radiation therapy for the endometrial cancer.

49. The method of any one of claims 1-8, wherein the cancer is esophageal cancer.

50. The method of claim 49, wherein the subject received prior systemic therapy and experienced disease progression on or after the systemic therapy.

51. The method of claim 50, wherein the subject received 1, 2 or 3 rounds of prior systemic therapy.

52. The method of anyone of claims 49-51, wherein the subject has been previously treated with one or more agents selected from the group consisting of a platinum-based therapy and a checkpoint inhibitor.

53. The method of any one of claims 49-52, wherein the subject has been previously treated with one or more agents selected from the group consisting of ramucirumab, paclitaxel, 5-fluorouracil, docetaxel, irinotecan, capecitabine and trastuzumab.

54. The method of any one of claims 49-53, wherein the subject has previously undergone surgery, radiation therapy or endoscopic mucosal resection for the esophageal cancer.

55. The method of any one of claims 1-8, wherein the cancer is prostate cancer.

56. The method of claim 55, wherein the subject received prior systemic therapy and experienced disease progression on or after the systemic therapy.

57. The method of claim 56, wherein the subject received 1, 2 or 3 rounds of prior systemic therapy.

58. The method of any one of claims 55-57, wherein the prostate cancer is castration-resistant prostate cancer.

59. The method of any one of claims 55-58, wherein the subject experienced bone metastases.

60. The method of any one of claims 55-59, wherein the subject has been previously treated with one or more agents selected from the group consisting of androgen deprivation therapy, a luteinizing hormone-releasing hormone agonist, a luteinizing hormone-releasing hormone antagonist, a CYP17 inhibitor, and an anti-androgen.

61. The method of any one of claims 55-60, wherein the subject has been previously treated with one or more agents selected from the group consisting of docetaxel, prednisone and cabazitaxel.

62. The method of any one of claims 55-61, wherein the subject has previously undergone surgery or radiation therapy for the prostate cancer.

63. The method of any one of claims 1-62, wherein the cancer is an advanced stage cancer.

64. The method of claim 63, wherein the advanced stage cancer is a stage 3 or stage 4 cancer.

65. The method of claim 63 or 64, wherein the advanced stage cancer is metastatic cancer.

66. The method of any one of claims 1-65, wherein the cancer is recurrent cancer.

67. The method of any one of claims 1-66, wherein the subject received prior treatment with standard of care therapy for the cancer and failed the prior treatment.

68. The method of any one of claims 1-67, wherein the monomethyl auristatin is monomethyl auristatin E (MMAE).

69. The method of any one of claims 1-68, wherein the anti-TF antibody or antigen-binding fragment thereof of the antibody-drug conjugate is a monoclonal antibody or a monoclonal antigen-binding fragment thereof.

70. The method of any one of claims 1-69, wherein the anti-TF antibody or antigen-binding fragment thereof of the antibody-drug conjugate comprises a heavy chain variable region and a light chain variable region, wherein the heavy chain variable region comprises:
(i) a CDR-H1 comprising the amino acid sequence of SEQ ID NO:1;
(ii) a CDR-H2 comprising the amino acid sequence of SEQ ID NO:2; and
(iii) a CDR-H3 comprising the amino acid sequence of SEQ ID NO:3; and
wherein the light chain variable region comprises:
(i) a CDR-L1 comprising the amino acid sequence of SEQ ID NO4;
(ii) a CDR-L2 comprising the amino acid sequence of SEQ ID NO:5; and
(iii) a CDR-L3 comprising the amino acid sequence of SEQ ID NO:6.

71. The method of any one of claims 1-70, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.

72. The method of any one of claims 1-71, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.

73. The method of any one of claims 1-72, wherein the anti-TF antibody of the antibody-drug conjugate is tisotumab.

74. The method of any one of claims 1-73, wherein the antibody-drug conjugate further comprises a linker between the anti-TF antibody or antigen-binding fragment thereof and the monomethyl auristatin.

75. The method of claim 74, wherein the linker is a cleavable peptide linker.

76. The method of claim 75, wherein the cleavable peptide linker has a formula: -MC-vc-PAB-, wherein:
a) MC is: 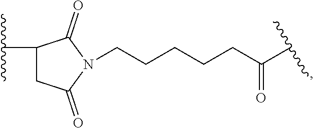 [SMILES: *C(=O)CCCCCN1C(=O)CC(*)C1=O]
b) vc is the dipeptide valine-citrulline, and
c) PAB is:

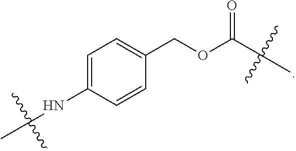 [SMILES: *Nc1ccc(COC(*)=O)cc1]

77. The method of any one of claims 74-76, wherein the linker is attached to sulphydryl residues of the anti-TF antibody obtained by partial reduction or full reduction of the anti-TF antibody or antigen-binding fragment thereof.

78. The method of claim 77, wherein the linker is attached to monomethyl auristatin E (MMAE), wherein the antibody-drug conjugate has the following structure: 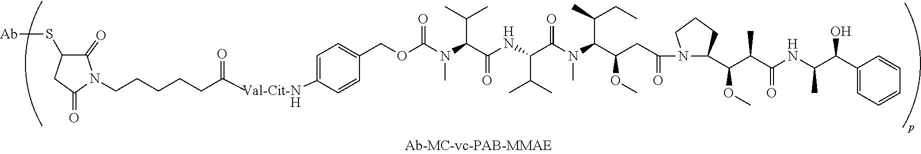
wherein p denotes a number from 1 to 8, S represents a sulphydryl residue of the anti-TF antibody, and Ab designates the anti-TF antibody or antigen-binding fragment thereof.

79. The method of claim 78, wherein the average value of p in a population of the antibody-drug conjugates is about 4.

80. The method of any one of claims 1-79, wherein the antibody-drug conjugate is tisotumab vedotin.

81. The method of any one of claims 1-80, wherein the route of administration for the antibody-drug conjugate is intravenous.

82. The method of anyone of claims 1-81, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express TF.

83. The method of any one of claims 1-82, wherein one or more therapeutic effects in the subject is improved after administration of the antibody-drug conjugate relative to a baseline.

84. The method of claim 83, wherein the one or more therapeutic effects is selected from the group consisting of: size of a tumor derived from the cancer, objective response rate, duration of response, time to response, progression free survival, overall survival and prostate specific antigen (PSA) level.

85. The method of any one of claims 55-62, wherein the subject exhibits a reduction in PSA level in a blood sample from the subject by at least about 5%, at least about 10%, at least about 15%, at least about 20%, at least about 25%, at least about 30%, at least about 35%, at least about 40%, at least about 45%, at least about 50%, at least about 60%, at least about 70%, or at least about 80% relative to the PSA level in a blood sample obtained from the subject before administration of the antibody-drug conjugate.

86. The method of any one of claims 1-85, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate.

87. The method of any one of claims 1-86, wherein the objective response rate is at least about 20%, at least about 25%, at least about 30%, at least about 35%, at least about 40%, at least about 45%, at least about 50%, at least about 60%, at least about 70%, or at least about 80%.

88. The method of any one of claims 1-87, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.

89. The method of any one of claims 1-88, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.

90. The method of any one of claims 1-89, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.

91. The method of any one of claims 1-90, wherein the subject has one or more adverse events and is further administered an additional therapeutic agent to eliminate or reduce the severity of the one or more adverse events.

92. The method of any one of claims 1-90, wherein the subject is at risk of developing one or more adverse events and is further administered an additional therapeutic agent to prevent or reduce the severity of the one or more adverse events.

93. The method of claim 91 or claim 92, wherein the one or more adverse events is anemia, abdominal pain, hypokalemia, hyponatremia, epistaxis, fatigue, nausea, alopecia, conjunctivitis, constipation, decreased appetite, diarrhea, vomiting, peripheral neuropathy, or general physical health deterioration.

94. The method of claim 91 or claim 92, wherein the one or more adverse events is a grade 3 or greater adverse event.

95. The method of claim 91 or claim 92, wherein the one or more adverse events is a serious adverse event.

96. The method of claim 91 or claim 92, wherein the one or more adverse events is conjunctivitis and/or keratitis and the additional agent is a preservative-free lubricating eye drop, an ocular vasoconstrictor and/or a steroid eye drop.

97. The method of any one of claims 1-96, wherein the antibody-drug conjugate is administered as a monotherapy.

98. The method of any one of claims 1-97, wherein the subject is a human.

99. The method of any one of claims 1-98, wherein the antibody-drug conjugate is in a pharmaceutical composition comprising the antibody-drug conjugate and a pharmaceutical acceptable carrier.

100. A kit comprising:
(a) a dosage ranging from about 0.9 mg/kg to about 2.1 mg/kg of an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof; and
(b) instructions for using the antibody drug conjugate according to the method of any one of claims 1-99.

101. Use of an antibody-drug conjugate that binds to tissue factor (TF) for the manufacture of a medicament for use in the method of any one of claims 1-99, wherein the antibody-drug conjugate comprises an anti-TF antibody or an antigen-binding fragment thereof conjugated to a monomethyl auristatin or a functional analog thereof or a functional derivative thereof.

102. An antibody-drug conjugate that binds to TF for use in the method of any one of claims 1-99, wherein the antibody-drug conjugate comprises an anti-TF antibody or an antigen-binding fragment thereof conjugated to a monomethyl auristatin or a functional analog thereof or a functional derivative thereof.


SEQUENCE LISTING (16 sequences)

SEQ ID NO: 1 - protein, 8 aa, Artificial Sequence
  Synthetic Construct
GFTFSNYA

SEQ ID NO: 2 - protein, 8 aa, Artificial Sequence
  Synthetic Construct
ISGSGDYT

SEQ ID NO: 3 - protein, 11 aa, Artificial Sequence
  Synthetic Construct
ARSPWGYYLDS

SEQ ID NO: 4 - protein, 6 aa, Artificial Sequence
  Synthetic Construct
QGISSR

SEQ ID NO: 5 - protein, 3 aa, Artificial Sequence
  Synthetic Construct
AAS

SEQ ID NO: 6 - protein, 9 aa, Artificial Sequence
  Synthetic Construct
QQYNSYPYT

SEQ ID NO: 7 - protein, 118 aa, Artificial Sequence
  Synthetic Construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 8 - protein, 107 aa, Artificial Sequence
  Synthetic Construct
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 9 - protein, 25 aa, Artificial Sequence
  Synthetic Construct
EVQLLESGGGLVQPGGSLRLSCAAS

SEQ ID NO: 10 - protein, 17 aa, Artificial Sequence
  Synthetic Construct
MSWVRQAPGKGLEWVSS

SEQ ID NO: 11 - protein, 38 aa, Artificial Sequence
  Synthetic Construct
YYTDSVKGRFTISRDNSKNTLYLQMNSLRAEDTAVYYC

SEQ ID NO: 12 - protein, 11 aa, Artificial Sequence
  Synthetic Construct
WGQGTLVTVSS

SEQ ID NO: 13 - protein, 26 aa, Artificial Sequence
  Synthetic Construct
DIQMTQSPPSLSASAGDRVTITCRAS

SEQ ID NO: 14 - protein, 17 aa, Artificial Sequence
  Synthetic Construct
LAWYQQKPEKAPKSLIY

SEQ ID NO: 15 - protein, 36 aa, Artificial Sequence
  Synthetic Construct
SLQSGVPSRFSGSGSGTDFTLTISSLQPEDFATYYC

SEQ ID NO: 16 - protein, 10 aa, Artificial Sequence
  Synthetic Construct
FGQGTKLEIK